(19) United States
(12) Patent Application Publication
(10) Pub. No.: US 2023/0314420 A1
(43) Pub. Date: Oct. 5, 2023

(54) ASSAYS FOR FIXED DOSE COMBINATIONS

(71) Applicants: Genentech, Inc., South San Francisco, CA (US); Hoffmann-La Roche Inc., Little Falls, NJ (US)

(72) Inventors: Cécile AVENAL, Illtal (FR); Nadine HOLZMANN, Basel (CH); Michael NOAK, Basel (CH); Tania RUCHTY, Werentzhouse (FR); Gabriele Maria SCHAEFER, San Mateo, CA (US); Franziska ZAEHRINGER, Neuenburg (DE)

(73) Assignees: Genentech, Inc., South San Francisco, CA (US); Hoffmann-La Roche Inc., Little Falls, NJ (US)

(21) Appl. No.: 18/153,234

(22) Filed: Jan. 11, 2023

Related U.S. Application Data
(63) Continuation of application No. PCT/EP2021/069405, filed on Jul. 13, 2021.
(60) Provisional application No. 63/051,596, filed on Jul. 14, 2020.

(30) Foreign Application Priority Data
Nov. 30, 2020 (EP) 20210641.5

(51) Int. Cl. G01N 33/543; C07K 16/32

(52) U.S. Cl. CPC G01N 33/543; C07K 16/32; G01N 2333/71; C07K 2317/24

(57) ABSTRACT

Assays to analyze quality and quantity attributes of fixed dose combinations are provided. In particular, assays for fixed dose combinations of two anti-HER2 antibodies, and for subcutaneous formulations comprising pertuzumab and trastuzumab are described herein.

### CROSS-REFERENCE TO RELATED APPLICATIONS

This application is a Continuation of International Application No. PCT/EP2021/069405, filed on Jul. 13, 2021 which claims benefit of priority to U.S. Application No. 63/051,596 filed Jul. 14, 2020 and European Application No. 20210641.5 filed Nov. 30, 2020 each of which is incorporated herein by reference in its entirety.

### SEQUENCE LISTING

This application contains a Sequence Listing which has been submitted electronically in XML format and is hereby incorporated by reference in its entirety. Said XML copy, created on Dec. 20, 2020, is named P36263US1SEQLIST and is 54,699 bytes in size.

### FIELD OF THE INVENTION

The invention concerns assays to analyze quality and quantity attributes of fixed dose combinations. In particular, the invention concerns assays for fixed dose combinations of two anti-HER2 antibodies, and for subcutaneous formulations comprising pertuzumab and trastuzumab.

### BACKGROUND OF THE INVENTION

To ensure the safety and efficacy of biopharmaceutical agents, product quality has to be continuously monitored. Before any product batch is released, certain distinct criteria including the critical quality attributes (CQA) have to be met. Critical quality attributes (CQA) are physical, chemical, biological or microbiological properties or characteristics that must be within an appropriate limit, range or distribution to ensure the desired product quality, safety and efficacy.

Potency tests, along with a number of other tests, are performed as part of product conformance testing, comparability studies, and stability testing. These tests are used to measure product attributes associated with product quality and manufacturing controls, and are performed to assure identity, purity, strength (potency), and stability of products used during all phases of clinical study. Similarly, potency measurements are used to demonstrate that only product lots that meet defined specifications or acceptance criteria are administered during all phases of clinical investigation and following market approval.

Ion-exchange chromatography (IEX) is widely used for the detailed characterization of therapeutic proteins and can be considered as a reference and powerful technique for the qualitative and quantitative evaluation of charge heterogeneity. IEX is typically a release method where specifications are set around the distribution of each acidic, main, and basic species specifically for monoclonal antibodies (mAbs). These charged species are considered product related impurities that may impact potency. Moreover, it is one of the few methods that can characterize the protein in its native confirmation as no denaturants are added. IEX may also be used as an identity method for certain biologics and is a routine test for stability and shelf-life justification.

Quantity is a CQA which is usually measured as protein content. It is critical for a biotechnological and biological product and should be determined using an appropriate assay, usually physicochemical in nature. For most biopharmaceutical agents, the protein content is measured by UV absorption.

Fixed dose combinations (FDC) combine two different active ingredients into a single dosage formulation. The combination of the two anti-HER2 antibodies trastuzumab and pertuzumab with a hyaluronidase enzyme is the first ever clinical development of a co-formulation of two highly similar monoclonal antibodies. The mechanisms of action of pertuzumab and trastuzumab are believed to complement each other as both bind to the HER2 receptor, but to different places. The combination of pertuzumab and trastuzumab is thought to provide a more comprehensive, dual blockade of the HER signaling pathways. The standard IV formulation of perjeta in combination with IV Herceptin and chemotherapy (the Perjeta-based regimen) is approved in over 100 countries for the treatment of both early and metastatic HER2-positive breast cancer. In the neoadjuvant early breast cancer (eBC) setting, the perjeta-based regimen has been shown to almost double the rate of pCR compared to Herceptin and chemotherapy. Additionally, the combination has been shown to significantly reduce the risk of recurrence of invasive disease or death in the adjuvant eBC setting. In the metastatic setting, the combination has shown an unprecedented survival benefit in previously untreated (first-line) patients with HER2-positive metastatic breast cancer.

The enzyme hyaluronidase in the FDC enables and optimizes SC drug delivery for appropriate co-administered therapeutics. The recombinant human hyaluronidase PH20 (rHuPH20) is an enzyme that temporarily degrades hyaluronan—a glycosaminoglycan or chain of natural sugars in the body, to aid in the dispersion and absorption of other injected therapeutic drugs.

Trastuzumab and pertuzumab have more than 93% sequence identity and differ only by 30 Da in total. Both antibodies have a molecular weight of approx. 148 kDa, and have almost the same isoelectric point. They bind the same target (HER2) and have a synergistic effect in vivo. Due to their structural and functional similarity, most of the usual analytical methods cannot be applied to this co-formulation.

### SUMMARY OF THE INVENTION

In one embodiment, a binding assay for a fixed dose combination (FDC) of two anti-HER2 antibodies is provided, comprising:
a. contacting the FDC with a capture reagent comprising a modified HER2 ECD subdomain;
b. contacting the sample with a detectable antibody;
c. quantifying the level of antibody bound to the capture reagent using a detection means for the detectable antibody.

In one embodiment the fixed dose combination comprises an antibody binding to HER2 extracellular subdomain II and an antibody binding to HER2 extracellular subdomain IV.

In one embodiment a binding assay for a fixed dose combination (FDC) of two anti-HER2 antibodies is provided, wherein the binding of an antibody binding to HER2 extracellular subdomain II is quantified.

In one embodiment the capture reagent comprises a recombinant HER2 extracellular domain II. In one embodiment the capture reagent comprises SEQ ID NO: 2 or SEQ ID NO: 23. In one embodiment the capture reagent comprises recombinant HER2 extracellular domains I, II, III. In one embodiment the capture reagent comprises SEQ ID NO: 24. In one embodiment the capture reagent does not comprise a HER2 subdomain IV.

In one embodiment a binding assay for a fixed dose combination (FDC) of two anti-HER2 antibodies is provided, wherein the binding of an antibody binding to HER2 subdomain II is quantified. In one embodiment the capture reagent comprises recombinant HER2 extracellular domain IV.

In one embodiment the capture reagent comprises SEQ ID NO: 4 or SEQ ID NO: 28. In one embodiment the capture reagent does not comprise a HER2 subdomain II In one embodiment the capture reagent comprises recombinant HER2 extracellular domains I, III, IV and domain II of EGFR. In one embodiment the capture reagent comprises SEQ ID NO. 29.

In one embodiment a binding assay for a fixed dose combination (FDC) of two anti-HER2 antibodies is provided, wherein the binding assay is for analyzing the biological activity of one of the anti-HER2 antibodies. In one embodiment the biological activity is quantified by correlating the level of antibody bound to the capture reagent with the biological activity of the isolated antibodies measured in a cell-based assay.

In one embodiment the capture reagent is coated on a microtiter plate. In one embodiment the detectable antibody targets the F(ab′)2 portion of the anti-HER2 antibody.

In one embodiment the fixed dose combination to be analyzed in the binding assay additionally comprises hyaluronidase.

In one embodiment an isolated protein comprising SEQ ID NO: 24 is provided. In one embodiment an isolated protein comprising SEQ ID NO: 29 is provided.

Further provided is a kit for specifically quantifying the binding of an antibody binding to HER2 extracellular subdomain II in a fixed dose combination (FDC) of a first antibody binding to HER2 extracellular subdomain II and a second anti-HER2 antibody, comprising:
a. a container containing, as a capture reagent, a protein comprising SEQ ID NO: 1, SEQ ID NO: 2 and SEQ ID NO: 34.
b. instructions for quantifying the binding of an antibody binding to HER2 extracellular subdomain II.

Further provided is a kit for specifically quantifying the binding of an antibody binding to HER2 extracellular subdomain IV in a fixed dose combination (FDC) of an antibody binding to HER2 extracellular subdomain IV and a second anti-HER2 antibody, the kit comprising:
a. a container containing, as a capture reagent, a protein comprising SEQ ID NO: 33, SEQ ID NO: 36, SEQ ID NO: 3 and SEQ ID NO: 4
b. instructions for quantifying the binding of an antibody binding to HER2 extracellular subdomain IV.

In another aspect of the invention, a method for evaluating a fixed dose composition comprising pertuzumab and trastuzumab is provided, said method comprising:
a. Binding the antibodies to a ion exchange material using a loading buffer, wherein the pH of the loading buffer is between about pH 7.5 and about pH 7.65.
b. Eluting the antibodies with an elution buffer, wherein the pH of the elution buffer is between about pH 7.5 and about pH 7.7.

In one embodiment, the ion exchange material is a cation exchange material. In one embodiment, the cation exchange chromatography material is a strong cation exchange material. In one embodiment, the cation exchange material comprises sulfonate groups.

In one embodiment step b is performed with a salt gradient. In one embodiment the elution buffer comprises sodium. In one embodiment, the elution buffer comprises sodium chloride.

In one embodiment the method for evaluating a fixed dose composition comprising pertuzumab and trastuzumab above additionally comprises step:
c. Selectively detecting charge variants of pertuzumab and trastuzumab in the composition.

In one embodiment the method is performed at a temperature of 32-40° C. In one embodiment the fixed dose combination of pertuzumab and trastuzumab to be analyzed additionally comprises hyaluronidase.

In one embodiment, a method for making a composition is provided, comprising: (1) producing a fixed dose composition comprising pertuzumab, trastuzumab and one or more variants thereof, and (2) subjecting the composition so-produced to an analytical assay to evaluate the amount of the variant(s) therein, wherein the variant(s) comprise: (i) pertuzumab deamidated at HC-Asn-391, pertuzumab FC sialic acid variant, and pertuzumab lysine glycation variant (ii) pertuzumab native antibody, (iii) trastuzumab native antibody (vi) trastuzumab with single isomerization of HC-Asp-102 to iso-aspartic acid at one heavy chain.

In one embodiment, a method for making a composition is provided, wherein the analytical assay of step (2) comprises:
a. Binding the antibodies to a ion exchange material using a loading buffer, wherein the pH of the loading buffer is between about pH 7.5 and about pH 7.65.
b. Eluting the antibodies with an elution buffer, wherein the pH of the elution buffer is between about pH 7.5 and about pH 7.7.

In one embodiment, the ion exchange material is a cation exchange material. In one embodiment, the cation exchange chromatography material is a strong cation exchange material. In one embodiment, the cation exchange material comprises sulfonate groups.

In one embodiment step b is performed with a salt gradient. In one embodiment the elution buffer comprises sodium. In one embodiment, the elution buffer comprises sodium chloride.

In one embodiment the analytical assay of step (2) additionally comprises step:
c. Selectively detecting charge variants of pertuzumab and trastuzumab in the composition.

In one embodiment the method is performed at a temperature of 32-40° C.

In one embodiment the fixed dose combination of pertuzumab and trastuzumab of step (1) additionally comprises hyaluronidase.

In one embodiment the fixed dose combination of pertuzumab and trastuzumab of step (1) comprises to 60 mg/mL Trastuzumab and 60-80 mg/mL Pertuzumab.

In one embodiment a composition comprising Pertuzumab and Trastuzumab is provided, wherein the composition comprises less than 23% of acidic pertuzumab variants selected from deamidation of HC-Asn-391, Fc sialic acid, and lysine glycation and trastuzumab variants deamidated at LC-Asn-30 and trastuzumab variants deamidated at HC-Asn-55, at least 28% of Pertuzumab native antibody, at least 16% of Trastuzumab native antibody and less than 12% trastuzumab with single isomerization of HC-Asp-102 to iso-aspartic acid at one heavy chain.

In one embodiment a composition comprising Pertuzumab and Trastuzumab is provided, wherein the composition comprises less than 23% of acidic pertuzumab variants selected from deamidation of HC-Asn-391, Fc sialic acid, and lysine glycation and trastuzumab variants deamidated at LC-Asn-30 and trastuzumab variants deamidated at HC-Asn-55, at least 38% of Pertuzumab native antibody, at least 16% of Trastuzumab native antibody and less than 9% trastuzumab with single isomerization of HC-Asp-102 to iso-aspartic acid at one heavy chain.

In one embodiment a composition comprising Pertuzumab and Trastuzumab is provided, wherein the composition comprises less than 21% of acidic pertuzumab variants selected from deamidation of HC-Asn-391, Fc sialic acid, and lysine glycation and trastuzumab variants deamidated at LC-Asn-30 and trastuzumab variants deamidated at HC-Asn-55, at least 28% of Pertuzumab native antibody, at least 23% of Trastuzumab native antibody and less than 12% trastuzumab with single isomerization of HC-Asp-102 to iso-aspartic acid at one heavy chain.

In one embodiment a composition comprising Pertuzumab and Trastuzumab is provided, wherein the composition comprises less than 23% peak area for the sum of peaks 1 to 3, at least 28% peak area for peak 4 (Pertuzumab native antibody), at least 16% peak area for peak 7 (Trastuzumab native antibody) and less than 12% peak area for peak 8 as determined by a method comprising the steps of:
a. Binding the antibodies to a ion exchange material using a loading buffer, wherein the pH of the loading buffer is between about pH 7.5 and about pH 7.65.
b. Eluting the antibodies with an elution buffer, wherein the pH of the elution buffer is between about pH 7.5 and about pH 7.7.

In one embodiment, the ion exchange material is a cation exchange material. In one embodiment, the cation exchange chromatography material is a strong cation exchange material. In one embodiment, the cation exchange material comprises sulfonate groups.

In one embodiment step b is performed with a salt gradient. In one embodiment the elution buffer comprises sodium. In one embodiment, the elution buffer comprises sodium chloride.

In one embodiment the method for evaluating a fixed dose composition comprising pertuzumab and trastuzumab above additionally comprises step:
c. Selectively detecting charge variants of pertuzumab and trastuzumab in the composition.

In one embodiment the method is performed at a temperature of 32-40° C. In one embodiment the composition comprising Pertuzumab and Trastuzumab additionally comprises rHuPH20.

In one embodiment the composition comprising Pertuzumab and Trastuzumab comprises 40 to 60 mg/mL Trastuzumab and 60-80 mg/mL Pertuzumab.

In one embodiment a composition comprising Pertuzumab and Trastuzumab is provided, wherein the composition comprises less than 23% peak area for the sum of peaks 1 to 3, at least 38% peak area for peak 4 (Pertuzumab native antibody), at least 16% peak area for peak 7 (Trastuzumab native antibody) and less than 9% peak area for peak 8 as determined in a method comprising the steps of:
a. Binding the antibodies to a ion exchange material using a loading buffer, wherein the pH of the loading buffer is between about pH 7.5 and about pH 7.65.
b. Eluting the antibodies with an elution buffer, wherein the pH of the elution buffer is between about pH 7.5 and about pH 7.7.

In one embodiment, the ion exchange material is a cation exchange material. In one embodiment, the cation exchange chromatography material is a strong cation exchange material. In one embodiment, the cation exchange material comprises sulfonate groups.

In one embodiment step b is performed with a salt gradient. In one embodiment the elution buffer comprises sodium. In one embodiment, the elution buffer comprises sodium chloride.

In one embodiment the method for evaluating a fixed dose composition comprising pertuzumab and trastuzumab above additionally comprises step:
c. Selectively detecting charge variants of pertuzumab and trastuzumab in the composition.

In one embodiment the method is performed at a temperature of 32-40° C. In one embodiment the composition comprising Pertuzumab and Trastuzumab additionally comprises rHuPH20.

In one embodiment the composition comprising Pertuzumab and Trastuzumab comprises 40 to 60 mg/mL Trastuzumab and 60-80 mg/mL Pertuzumab.

In one embodiment a composition comprising Pertuzumab and Trastuzumab is provided, wherein the composition comprises less than 21% peak area for the sum of peaks 1 to 3, at least 28% peak area for peak 4 (Pertuzumab native antibody), at least 23% peak area for peak 7 (Trastuzumab native antibody) and less than 12% peak area for peak 8 as determined in a method comprising the steps of:
a. Binding the antibodies to a ion exchange material using a loading buffer, wherein the pH of the loading buffer is between about pH 7.5 and about pH 7.65.
b. Eluting the antibodies with an elution buffer, wherein the pH of the elution buffer is between about pH 7.5 and about pH 7.7.

In one embodiment, the ion exchange material is a cation exchange material. In one embodiment, the cation exchange chromatography material is a strong cation exchange material. In one embodiment, the cation exchange material comprises sulfonate groups.

In one embodiment step b is performed with a salt gradient. In one embodiment the elution buffer comprises sodium. In one embodiment, the elution buffer comprises sodium chloride.

In one embodiment the method for evaluating a fixed dose composition comprising pertuzumab and trastuzumab above additionally comprises step:
c. Selectively detecting charge variants of pertuzumab and trastuzumab in the composition.

In one embodiment the method is performed at a temperature of 32-40° C. In one embodiment the composition comprising Pertuzumab and Trastuzumab additionally comprises rHuPH20.

In one embodiment the composition comprising Pertuzumab and Trastuzumab comprises 40 to 60 mg/mL Trastuzumab and 60-80 mg/mL Pertuzumab.

In a further aspect of the invention the compositions provided herein are obtainable by a method comprising the following steps:
a. adding a pre-defined amount of pertuzumab to a compounding vessel
b. adding trastuzumab in a 1:1 Trastuzumab to Pertuzumab ratio or in a 1:2 Trastuzumab to Pertuzumab ratio
c. adding rHuPH20.

In a further aspect, a method for analyzing the protein content of a fixed dose combination (FDC) of two anti-HER2 antibodies is provided, comprising
a. Providing a RP-HPLC phenyl column;
b. Loading the fixed dose combination (FDC) of two anti-HER2 antibodies on the RP-HPLC column;
c. Separating the two anti-HER2 antibodies at a flow rate of 0.2-0.4 mL/min, wherein the column temperature is 64° C. to 76° C.

In one embodiment the fixed dose combination comprises Pertuzumab and Trastuzumab. In one embodiment the fixed dose combination of Pertuzumab and Trastuzumab additionally comprises hyaluronidase.

In one embodiment of the method for analyzing the protein content of a fixed dose combination (FDC) of two anti-HER2 antibodies, the separation in step c) is achieved with a water—2-propanol/acetonitrile gradient.

In one embodiment of the method for analyzing the protein content of a fixed dose combination (FDC) of two anti-HER2 antibodies, the flow rate in step c) is about 0.3 mL/min.

In one embodiment of the method for analyzing the protein content of a fixed dose combination (FDC) of two anti-HER2 antibodies, the antibodies are separated over 10 to 20 minutes. In one such embodiment, the antibodies are separated over 15 minutes. In one embodiment the antibodies are separated over 15 minutes at a flow rate of 0.3 mL/min.

In one embodiment of the method for analyzing the protein content of a fixed dose combination (FDC) of two anti-HER2 antibodies, the column temperature is 70° C.+−2° C.

In one embodiment of the method for analyzing the protein content of a fixed dose combination (FDC) of two anti-HER2 antibodies, the phenyl column is a column selected from the group of Agilent Zorbax RRHD 300-Diphenyl column, Acclaim Phenyl-1 (Dionex), Pursuit® XRs Diphenyl, Pinnacle® Biphenyl, Zorbax® Eclipse® Plus Hexyl Phenyl, Ascentis Phenyl, and Agilent AdvanceBio RP mAb Diphenyl.

### BRIEF DESCRIPTION OF THE DRAWINGS

FIG. 1 provides a schematic of the HER2 protein structure, and amino acid sequences for Domains I-IV (SEQ ID Nos. 1-4, respectively) of the extracellular domain thereof. FIGS. 2A and 2B depict alignments of the amino acid sequences of the variable light (V_{L}) (FIG. 2A) and variable heavy (V_{H}) (FIG. 2B) domains of murine monoclonal antibody 2C4 (SEQ ID Nos. 5 and 6, respectively); V_{L} and V_{H} domains of variant 574/pertuzumab (SEQ ID NOs. 7 and 8, respectively), and human V_{L} and V_{H} consensus frameworks (hum id, light kappa subgroup I; humIII, heavy subgroup III) (SEQ ID Nos. 9 and 10, respectively). Asterisks identify differences between variable domains of pertuzumab and murine monoclonal antibody 2C4 or between variable domains of pertuzumab and the human framework. Complementarity Determining Regions (CDRs) are in brackets.

FIGS. 3A and 3B show the amino acid sequences of pertuzumab light chain (FIG. 3A; SEQ ID NO. 11) and heavy chain (FIG. 3B; SEQ ID No. 12). CDRs are shown in bold. Calculated molecular mass of the light chain and heavy chain are 23,526.22 Da and 49,216.56 Da (cysteines in reduced form). The carbohydrate moiety is attached to Asn 299 of the heavy chain.

FIGS. 4A and 4B show the amino acid sequences of trastuzumab light chain (FIG. 4A; SEQ ID NO. 13) and heavy chain (FIG. 4B; SEQ ID NO. 14), respectively. Boundaries of the variable light and variable heavy domains are indicated by arrows.

FIGS. 5A and 5B depict a variant pertuzumab light chain sequence (FIG. 5A; SEQ ID NO. 15) and a variant pertuzumab heavy chain sequence (FIG. 5B; SEQ ID NO. 16), respectively.

FIG. 6 depicts a schematic of the HER2 extracellular domain and the capture reagents useful in the ELISA assay described herein. P-HER2 variant: modified HER2 ECD for analyzing pertuzumab potency. T-HER2 variant: modified HER2 ECD for analyzing trastuzumab potency.

FIGS. 7A and 7B depict the selective sensitivity of cell-based assays. FIG. 7A: Pertuzumab anti-proliferation assay using MDA-MB-175 VII cells. FIG. 7B: Trastuzumab anti-proliferation assay using BT-474 cells.

FIGS. 8A and 8B depict complementary mechanisms of pertuzumab and trastuzumab in the cell-based anti-proliferation assays. FIG. 8A: Pertuzumab anti-proliferation assay: Upon addition of trastuzumab in a 1:1 ratio, the dose-response curve shifts towards lower concentration. FIG. 8B: Trastuzumab anti-proliferation assay: Upon addition of pertuzumab in a 1:1 ratio, the dose-response curve slightly shifts towards lower concentration.

FIGS. 9A and 9B depict the masking effect of the cell-based anti-proliferation assays. FIG. 9A: Pertuzumab anti-proliferation assay: Greatly reduced affinity of pertuzumab mutant (HC S55A) to HER2 (solid symbols); masking of pertuzumab mutant affinity loss upon addition of trastuzumab (open symbols). FIG. 9B: Trastuzumab anti-proliferation assay: Greatly reduced affinity of trastuzumab mutant (LC H91A) to HER2 (solid symbols); masking of trastuzumab mutant affinity loss upon addition of pertuzumab (open symbols).

FIG. 10 depicts a representative dose-response curve of the pertuzumab ELISA.

FIG. 11 depicts a representative dose-response curve of the trastuzumab ELISA.

FIG. 12 shows a representative chromatogram of the IEC method provided therein to analyze the pertuzumab trastuzumab FDC charge variants.

FIG. 13 depicts IE-HPLC chromatograms of pertuzumab trastuzumab FDC drug product, pertuzumab and trastuzumab.

FIG. 14A and FIG. 14B show HER2 affinity mutants in the ELISAs. FIG. 14A: Pertuzumab ELISA: Greatly reduced binding activity of pertuzumab mutant (HC S55A) to HER2 (open symbols) compared to pertuzumab (solid symbols). FIG. 14B: Trastuzumab ELISA: Greatly reduced affinity of trastuzumab mutant (LC H91A) to HER2 (open symbols) compared to trastuzumab (solid symbols).

FIG. 15 depicts an example RP-UHPLC chromatogram to analyze protein content of FDC LD Reference Standard.

FIG. 16 depicts example RP-UHPLC chromatogram to analyze protein content of FDC MD Reference Standard.

### DETAILED DESCRIPTION OF THE PREFERRED EMBODIMENTS

### I. Definitions

The term “about” as used in the present patent specification is meant to specify that the specific value provided may vary to a certain extent, such as e.g. means that variations in the range of +10%, are included in the given value. In one embodiment, the variations in the range of +/−5% are included in the given value.

A “HER receptor” is a receptor protein tyrosine kinase which belongs to the HER receptor family and includes EGFR, HER2, HER3 and HER4 receptors. The HER receptor will generally comprise an extracellular domain, which may bind an HER ligand and/or dimerize with another HER receptor molecule; a lipophilic transmembrane domain; a conserved intracellular tyrosine kinase domain; and a carboxyl-terminal signaling domain harboring several tyrosine residues which can be phosphorylated. The HER receptor may be a “native sequence” HER receptor or an “amino acid sequence variant” thereof. Preferably the HER receptor is native sequence human HER receptor.

The expressions “ErbB2” and “HER2” are used interchangeably herein and refer to human HER2 protein described, for example, in Semba et al., PNAS (USA) 82:6497-6501 (1985) and Yamamoto et al. Nature 319:230-234 (1986) (Genebank accession number X03363). The term “erbB2” refers to the gene encoding human ErbB2 and “neu” refers to the gene encoding rat p185^{neu}. Preferred HER2 is native sequence human HER2.

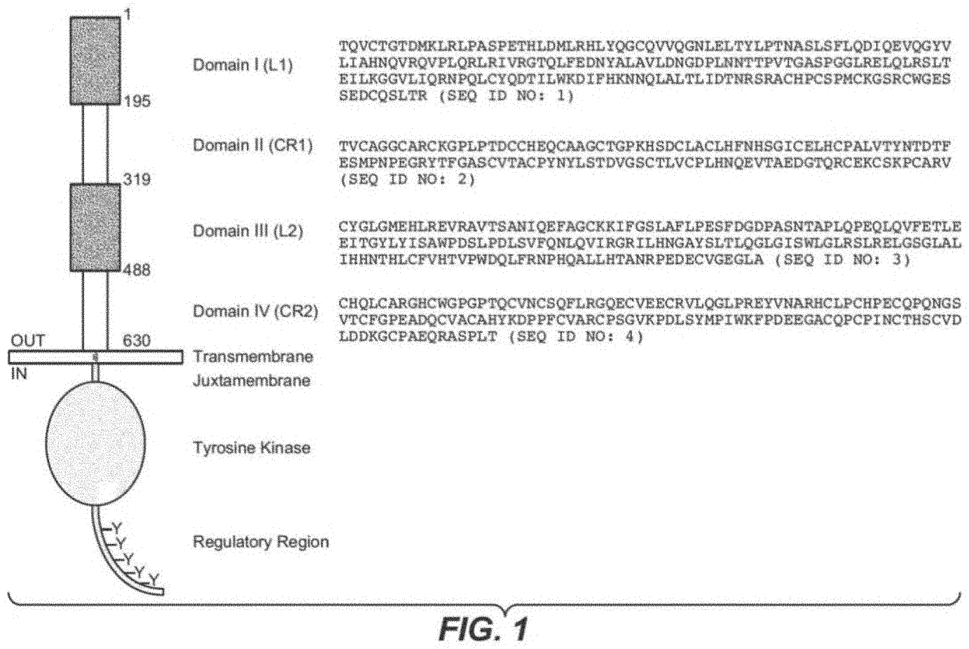

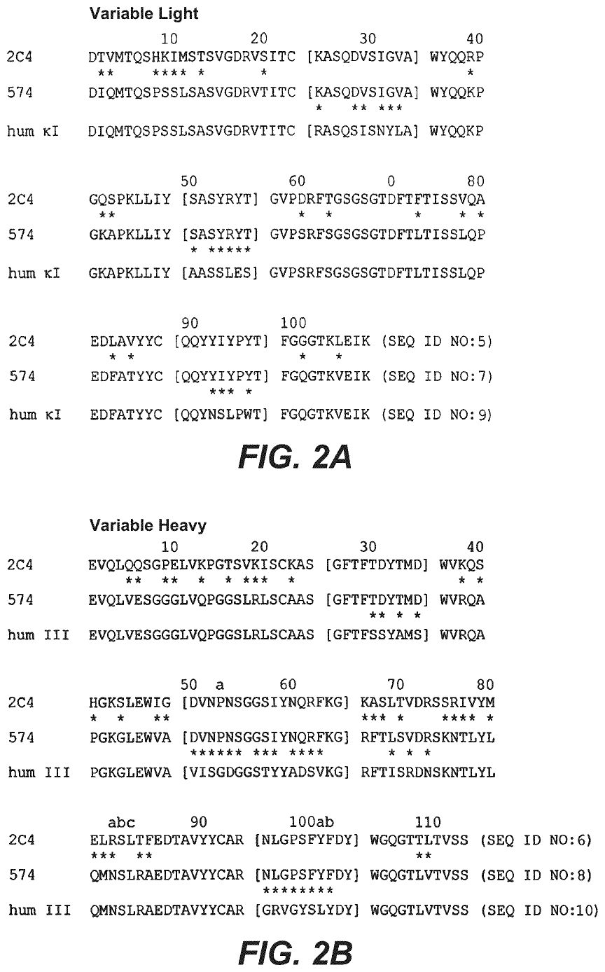

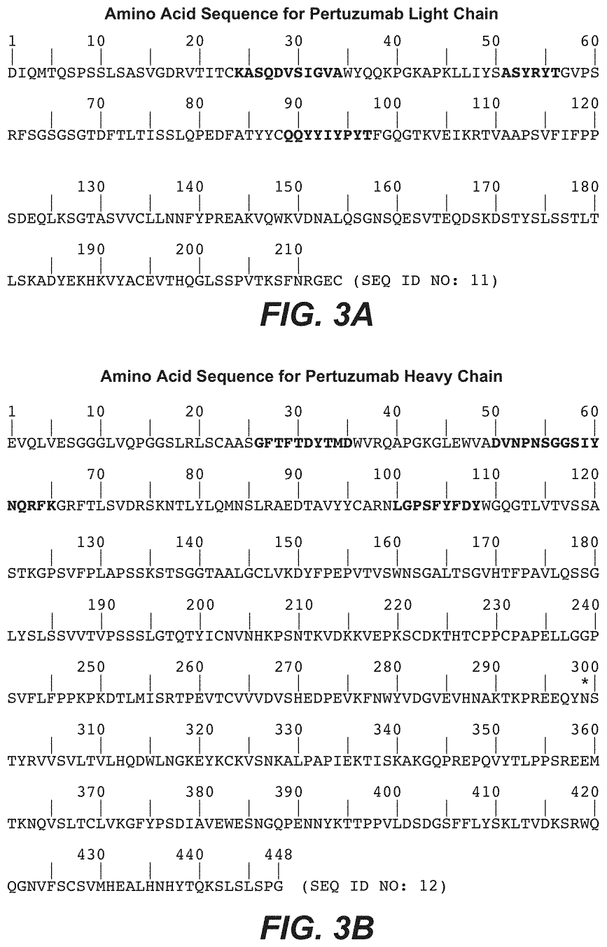

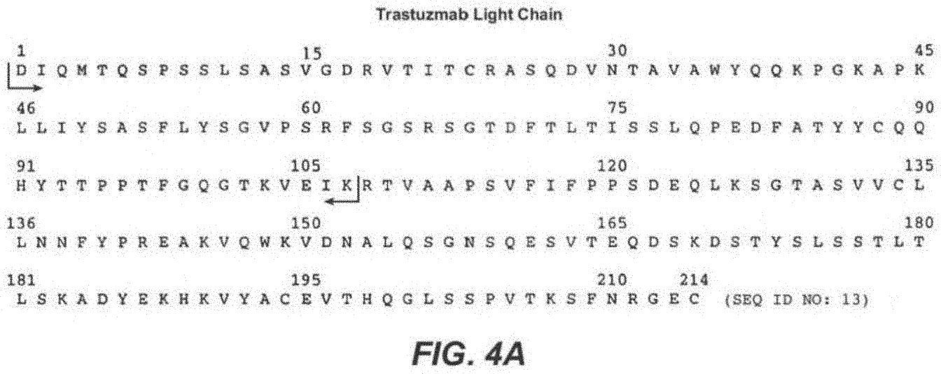

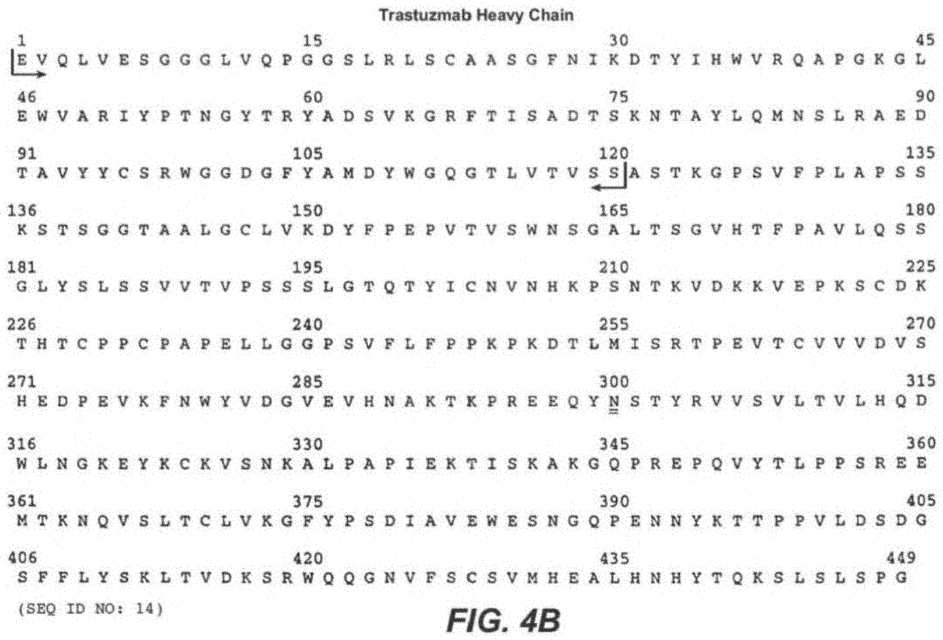

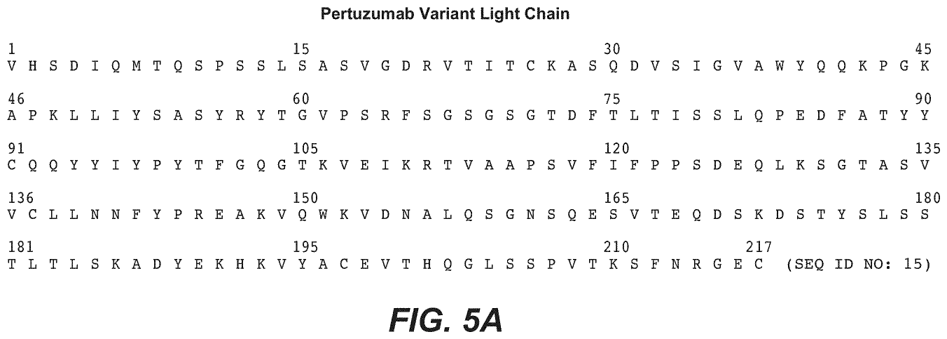

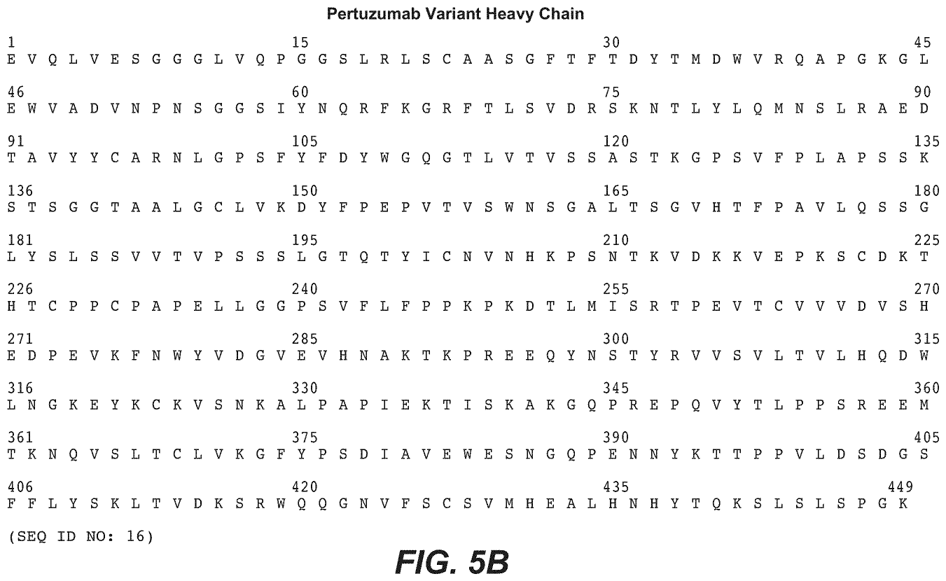

Herein, “HER2 extracellular domain” or “HER2 ECD” refers to a domain of HER2 that is outside of a cell, either anchored to a cell membrane, or in circulation, including fragments thereof. The amino acid sequence of HER2 is shown in FIG. 1. In one embodiment, the extracellular domain of HER2 may comprise four subdomains: “subdomain I” (amino acid residues from about 1-195; SEQ ID NO:1), “subdomain II” (amino acid residues from about 196-319; SEQ ID NO:2), “subdomain III” (amino acid residues from about 320-488: SEQ ID NO:3), and “subdomain IV” (amino acid residues from about 489-630; SEQ ID NO:4) (residue numbering without signal peptide). See Garrett et al. Mol. Cell. 11: 495-505 (2003), Cho et al. Nature 421: 756-760 (2003), Franklin et al. Cancer Cell 5:317-328 (2004), and Plowman et al. Proc. Natl. Acad. Sci. 90:1746-1750 (1993), as well as FIG. 1 herein. A “recombinant HER2 extracellular subdomain” or “recombinant HER2 ECD subdomain” comprises the full-length or a truncated version of the respective native HER2 ECD subdomain. In order for the conformation of the modified HER2 ECD to resemble the conformation of the native HER2 ECD as closely as possible, the recombinant HER2 ECD subdomains can be truncated by up to six amino acids, preferably at their C-terminus.

An “anti-HER2 antibody” or “HER2 antibody” is an antibody that binds to the HER2 receptor. Optionally, the HER2 antibody further interferes with HER2 activation or function. Anti-HER2 antibodies of interest herein are pertuzumab and trastuzumab.

An antibody that “binds to extracellular subdomain II” of HER2 binds to residues in domain II (SEQ ID NO: 2) and optionally residues in other subdomain(s) of HER2, such as subdomains I and III (SEQ ID NOs: 1 and 3, respectively). Preferably, the antibody that binds to extracellular subdomain II binds to the junction between extracellular subdomains I, II and III of HER2. In one embodiment, the antibody that binds extracellular subdomain II is pertuzumab or a variant thereof.

For the purposes herein, “pertuzumab” and “rhuMAb 2C4”, which are used interchangeably, refer to an antibody comprising the variable light and variable heavy amino acid sequences in SEQ ID NOs: 7 and 8, respectively. Where pertuzumab is an intact antibody, it preferably comprises an IgG1 antibody; in one embodiment comprising the light chain amino acid sequence in SEQ ID NO: 11 or 15, and heavy chain amino acid sequence in SEQ ID NO: 12 or 16. The antibody is optionally produced by recombinant Chinese Hamster Ovary (CHO) cells. The terms “pertuzumab” and “rhuMAb 2C4” herein cover biosimilar versions of the drug with the United States Adopted Name (USAN) or International Nonproprietary Name (INN): pertuzumab.

An antibody that “binds to extracellular subdomain IV” of HER2 binds to residues in domain IV (SEQ ID NO: 4) and optionally residues in other subdomain(s) of HER2. In one embodiment the antibody that binds extracellular subdomain IV is trastuzumab or a variant thereof.

For the purposes herein, “trastuzumab” and rhuMAb4D5”, which are used interchangeably, refer to an antibody comprising the variable light and variable heavy amino acid sequences from within SEQ ID Nos: 13 and 14, respectively. Where trastuzumab is an intact antibody, it preferably comprises an IgG1 antibody; in one embodiment comprising the light chain amino acid sequence of SEQ ID NO: 13 and the heavy chain amino acid sequence of SEQ ID NO: 14. The antibody is optionally produced by Chinese Hamster Ovary (CHO) cells. The terms “trastuzumab” and “rhuMAb4D5” herein cover biosimilar versions of the drug with the United States Adopted Name (USAN) or International Nonproprietary Name (INN): trastuzumab.

The term “co-formulation” is used herein to refer to a single ready-to-use pharmaceutical formulation comprising two or more active ingredients, including, for example, a single ready-to-use pharmaceutical formulation comprising pertuzumab and trastuzumab formulated together for subcutaneous (SC) administration.

A “Fixed Dose Combination” or “FDC” is used herein to refer to a single ready-to-use pharmaceutical formulation comprising two or more active ingredients, including, for example, a single ready-to-use pharmaceutical formulation comprising pertuzumab and trastuzumab formulated together for subcutaneous (SC) administration. A “pertuzumab trastuzumab FDC” comprises pertuzumab, trastuzumab and optionally hyaluronidase.

The term “hyaluronidase” or “hyaluronidase enzyme” refers to a group of generally neutral- or acid-active enzymes found throughout the animal kingdom. Hyaluronidases vary with respect to substrate specificity, and mechanism of action (WO 2004/078140). There are three general classes of hyaluronidases: 1. Mammalian-type hyaluronidases, (EC 3.2.1.35) which are endo-β-N-acetylhexosaminidases with tetrasaccharides and hexasaccharides as the major end products. They have both hydrolytic and transglycosidase activities, and can degrade hyaluronan and chondroitin sulfates (CS), generally C4-S and C6-S. 2. Bacterial hyaluronidases (EC 4.2.99.1) degrade hyaluronan and, and to various extents, CS and DS. They are endo-β-N-acetylhexosaminidases that operate by a beta elimination reaction that yields primarily disaccharide end products. 3. Hyaluronidases (EC 3.2.1.36) from leeches, other parasites, and crustaceans are endo-beta-glucuronidases that generate tetrasaccharide and hexasaccharide end products through hydrolysis of the β1-3 linkage. Mammalian hyaluronidases can be further divided into two groups: neutral-active and acid-active enzymes. The hyaluronidase-like enzymes can also be characterized by those which are generally locked to the plasma membrane via a glycosylphosphatidyl inositol anchor such as human HYAL2 and human PH20 [Danilkovitch-Miagkova et al., Proc. Natl. Acad. Sci. USA, 2003; 100(8):4580-4585; Phelps et al., Science 1988; 240(4860): 1780-1782], and those which are generally soluble such as human HYAL1 [Frost, I. G. et al., “Purification, cloning, and expression of human plasma hyaluronidase”, Biochem. Biophys. Res. Commun. 1997; 236(1):10-15]. Bovine PH20 is very loosely attached to the plasma membrane and is not anchored via a phospholipase sensitive anchor [Lalancette et al., Biol. Reprod., 2001; 65(2):628-36]. This unique feature of bovine hyaluronidase has permitted the use of the soluble bovine testes hyaluronidase enzyme as an extract for clinical use (Wydase™, Hyalase™). Other PH20 species are lipid anchored enzymes that are generally not soluble without the use of detergents or lipases. For example, human PH20 is anchored to the plasma membrane via a GPI anchor. Naturally occurring macaque sperm hyaluronidase is found in both a soluble and membrane bound form. While the 64 kDa membrane bound form possesses enzyme activity at pH 7.0, the 54 kDa form is only active at pH 4.0 [Cherr et al., Dev. Biol., 1996; 10; 175(1): 142-53]. WO2006/091871 describes soluble hyaluronidase glycoproteins (sHASEGPs) which facilitate the administration of therapeutic drug into the hypodermis. By rapidly depolymerizing HA in the extracellular space sHASEGP reduces the viscosity of the interstitium, thereby increasing hydraulic conductance and allowing for larger volumes to be administered safely and comfortably into the SC tissue. The preferred hyaluronidase enzyme is a human hyaluronidase enzyme, most preferably the recombinant human hyaluronidase enzyme known as rHuPH20 (vorhyaluronidase alfa). rHuPH20 is a member of the family of neutral and acid-active β-1,4 glycosyl hydrolases that depolymerize hyaluronan by the hydrolysis of the β-1,4 linkage between the C1 position of N-acetyl glucosamine and the C4 position of glucuronic acid. Hyaluronidase products approved in EU countries include Hylase® “Dessau” and Hyalase®. Hyaluronidase products of animal origin approved in the US include Vitrase™, Hydase™, and Amphadase™.

rHuPH20 is the first and only recombinant human hyaluronidase enzyme currently available for therapeutic use. The amino acid sequence of rHuPH20 (HYLENEX™) is well known and available under CAS Registry No. 75971-58-7. The approximate molecular weight is 61 kDa. In one embodiment, the pertuzumab trastuzumab FDC comprises hyaluronidase, optionally at a concentration of 2000 U/mL.

A “loading” dose herein generally comprises an initial dose of a therapeutic agent administered to a patient, and is followed by one or more maintenance dose(s) thereof. The loading dose (LD) of the pertuzumab trastuzumab FDC comprises 40 mg/mL trastuzumab, 80 mg/mL pertuzumab and 2000 U/mL rHuPH20.

A “maintenance” dose herein refers to one or more doses of a therapeutic agent administered to the patient over a treatment period. Usually, the maintenance doses are administered at spaced treatment intervals, such as approximately every week, approximately every 2 weeks, approximately every 3 weeks, or approximately every 4 weeks, preferably every 3 weeks. The maintenance dose (MD) of the pertuzumab trastuzumab FDC comprises 60 mg/mL trastuzumab, 60 mg/mL pertuzumab and 2000 U/mL rHuPH20.

As used herein, “a capture reagent” refers to any agent that is capable of binding to an analyte (e.g., an anti-HER2 antibody). Preferably, “a capture reagent” refers to any agent that is specifically bound by an anti-HER2 antibody in a fixed dose combination of two anti-HER2 antibodies. To specifically analyze the binding of one of the two anti-HER2 antibodies in the fixed dose combination the capture reagent must be specific for that antibody; e.g., the antibody to be analyzed should have a higher binding affinity and/or specificity to the capture reagent than the second anti-HER2 antibody of the FDC. In one embodiment the capture reagent in the assays provided is a modified HER2 ECD.

A “modified HER2 ECD” is a genetically engineered protein or peptide that comprises one or more recombinant HER2 ECD subdomains. The HER2 ECD is modified such that one of the anti-HER2 antibodies to be assessed in the FDC can bind while the second anti-HER2 antibody in the FDC will not bind to it. This is achieved by either omitting the HER2 ECD subdomain to which the second anti-HER2 antibody binds to or by replacing it by a structurally close subdomain that is not bound to by either of the anti-HER2 antibodies. Preferably the modified HER2 ECD is constructed to mimic the native HER2 ECD as closely as possible. The subdomains can be full-length or shortened by a few amino acids at the N or C-terminus. It has been found by the inventors of the present invention that the integrity of the three-dimensional structure of the HER2 ECD is retained or improved when using one or more recombinant HER2 ECD subdomains that are shortened by about 4 to 5 amino acids at the C-terminus.

“Fc domain” herein is used to define a C-terminal domains of an immunoglobulin heavy chain. The Fc domain may of various origin, e.g. murine, rat, goat or human origin. Although the boundaries of the Fc region of an immunoglobulin heavy chain might vary, the human IgG heavy chain Fc domain is usually defined to stretch from an amino acid residue at position Cys226, or from Pro230, to the carboxyl-terminus thereof. Unless indicated otherwise, herein the numbering of the residues in an immunoglobulin heavy chain is that of the EU index as in Kabat et al., Sequences of Proteins of Immunological Interest, 5th Ed. Public Health Service, National Institutes of Health, Bethesda, MD (1991), expressly incorporated herein by reference. The “EU index as in Kabat” refers to the residue numbering of the human IgG1 EU antibody.

The term “detectable antibody,” as used herein, refers to an antibody that is linked to an agent or detectable label that is capable of generating a detectable signal, which can be used to assess the presence and/or quantity of the analyte (i.e. anti-HER 2 antibody) to be detected. The terms “label” or “detectable label” is any chemical group or moiety that can be linked to the detectable antibody. Examples of detectable labels include luminescent labels (e.g., fluorescent, phosphorescent, chemiluminescent, bioluminescent and electrochemiluminescent labels), radioactive labels, enzymes, particles, magnetic substances, electroactive species and the like. Alternatively, a detectable label may signal its presence by participating in specific binding reaction. Examples of such labels include haptens, antibodies, biotin, streptavidin, his-tag, nitrilotriacetic acid, glutathione S-transferase, glutathione and the like.

The term “detection means” refers to a moiety or technique used to detect the presence of the detectable antibody through signal reporting that is then read out in the assay herein. “Photoluminescence” is the process whereby a material luminesces subsequent to the absorption by that material of light (alternatively termed electromagnetic radiation or emr). Fluorescence and phosphorescence are two different types of photoluminescence. “Chemiluminescent” processes entail the creation of the luminescent species by a chemical reaction. “Electro-chemiluminescence” or “ECL” is the process whereby a species, e.g., antibody of interest, luminesces upon the exposure of that species to electrochemical energy in an appropriate surrounding chemical environment.

As used herein, the term “ELISA” (also known as Enzyme-linked immunosorbent assay) refers to a biochemical technique used mainly to detect the presence of an antibody in a biological sample. For purposes of this application, the ELISA technique is used for the detection and quantification of an anti-HER2 antibody in a Fixed Dose Combination. Typically for ELISA based assays, the capture reagent is immobilized or immobilizable.

Herein, “potency” refers to the therapeutic activity or intended biological effect of a biotherapeutic drug. Potency of a biotherapeutic drug can be determined by measuring or quantifying the biological activity of the active ingredient of said biotherapeutic drug.

Herein, “biological activity” of a monoclonal antibody refers to the ability of the antibody to bind to an antigen and result in a measurable biological response, which can be measured in vitro or in vivo. In one embodiment, the biological activity refers to the ability to bind to the capture agent in the binding assay as provided herein. In one embodiment the binding of the anti-HER2 antibody in the FDC is correlated to ability of the anti-HER2 antibody in a single—antibody formulation to inhibit proliferation in a human breast cancer cell line. A suitable human breast cancer cell line for testing pertuzumab is MDA-MB-175-VII. A suitable human breast cancer cell line for testing trastuzumab is BT-474.

The term “antibody” herein is used in the broadest sense and specifically covers monoclonal antibodies, polyclonal antibodies, multispecific antibodies (e.g. bispecific antibodies), and antibody fragments, so long as they exhibit the desired biological activity.

“Humanized” forms of non-human (e g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992) Humanized HER2 antibodies specifically include trastuzumab (HERCEPTIN®) as described in Table 3 of U.S. Pat. No. 5,821,337 expressly incorporated herein by reference and as defined herein; and humanized 2C4 antibodies such as pertuzumab as described and defined herein.

An “intact antibody” herein is one, which comprises two antigen binding regions, and an Fc region. Preferably, the intact antibody has a functional Fc region.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragment(s).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

A “naked antibody” is an antibody that is not conjugated to a heterologous molecule, such as a cytotoxic moiety or radiolabel.

An “affinity matured” antibody is one with one or more alterations in one or more hypervariable regions thereof, which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by V_{H} and V_{L} domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “vial” is a container suitable for holding a liquid or lyophilized preparation. In one embodiment, the vial is a single-use vial, e.g. a 10 mL or a 20 mL single-use vial with a stopper, such as a 10 mL single use glass vial with a 20 mm stopper.

As used herein, “eluting” refers to removing a protein of interest (e.g., an antibody) from a cation exchange material, by altering the ionic strength of the buffer surrounding the cation exchange material such that the buffer competes with the molecule for the charged sites on the ion exchange material.

As used herein the term “chromatography” refers to the process by which a solute of interest, e.g., a protein of interest, in a mixture is separated from other solutes in the mixture by percolation of the mixture through an adsorbent, which adsorbs or retains a solute more or less strongly due to properties of the solute, such as pi, hydrophobicity, size and structure, under particular buffering conditions of the process.

The terms “ion-exchange” and “ion-exchange chromatography” refer to a chromatographic process in which an ionizable solute of interest (e.g., the antibodies of the FDC and their acidic and basic variants) interacts with an oppositely charged ligand linked (e.g., by covalent attachment) to a solid phase ion exchange material under appropriate conditions of pH and conductivity, such that the solute of interest interacts non-specifically with the charged compound more or less than the solute impurities or contaminants in the mixture.

“Ion-exchange chromatography” specifically includes cation exchange (CEX), anion exchange, and mixed mode chromatographies.

A “cation exchange material” or “CEX material” refers to a solid phase which is negatively charged, and which has free cations for exchange with cations in an aqueous solution passed over or through the solid phase. Any negatively charged ligand attached to the solid phase suitable to form the cation exchange material can be used, e.g., a carboxylate, sulfonate and others as described below. Commercially available cation exchange materials include, but are not limited to, for example, those having a sulfonate based group (e.g., MonoS, MiniS, Source 15S and 30S, SP Sepharose Fast Flow™, SP Sepharose High Performance from GE Healthcare, Toyopearl SP-650S and SP-650M from Tosoh, Macro-Prep High S from BioRad, Ceramic HyperD S, Trisacryl M and LS SP and Spherodex LS SP from Pall Technologies); a sulfoethyl based group (e.g., Fractogel SE, from EMD, Poros S-10 and S-20 from Applied Biosystems); a sulphopropyl based group (e.g., TSK Gel SP 5PW and SP-5PW-HR from Tosoh, Poros HS-20 and HS 50 from Applied Biosystems); a sulfoisobutyl based group (e.g., (Fractogel EMD SO3 “from EMD); a sulfoxyethyl based group (e.g., SE52, SE53 and Express-Ion S from Whatman), a carboxymethyl based group (e.g., CM Sepharose Fast Flow from GE Healthcare, Hydrocell CM from Biochrom Labs Inc., Macro-Prep CM from BioRad, Ceramic HyperD CM, Trisacryl M CM, Trisacryl LS CM, from Pall Technologies, Matrx Cellufine C500 and C200 from Millipore, CM52, CM32, CM23 and Express-Ion C from Whatman, Toyopearl CM-650S, CM-650M and CM-650C from Tosoh); sulfonic and carboxylic acid based groups (e.g., BAKERBOND Carboxy-Sulfon from J.T. Baker); a carboxylic acid based group (e.g., WP CBX from J.T Baker, DOWEX MAC-3 from Dow Liquid Separations, Amberlite Weak Cation Exchangers, DOWEX Weak Cation Exchanger, and Diaion Weak Cation Exchangers from Sigma-Aldrich and Fractogel EMD COO— from EMD); a sulfonic acid based group (e.g., Hydrocell SP from Biochrom Labs Inc., DOWEX Fine Mesh Strong Acid Cation Resin from Dow Liquid Separations, UNOsphere S, WP Sulfonic from J. T. Baker, Sartobind S membrane from Sartorius, Amberlite Strong Cation Exchangers, DOWEX Strong Cation and Diaion Strong Cation Exchanger from Sigma-Aldrich); and a orthophosphate based group (e.g., PI 1 from Whatman).

Depending on the chemical nature of the charged group/substituent the “ion exchange chromatography material” can be classified as strong or weak ion exchange material, depending on the strength of the covalently bound charged substituent. A “strong cation exchange material” or “(SCX) material” as used herein has a sulfonic acid based group, e.g. sulfonate, sulfopropyl group, sodium polystyrene sulfonate or polyAMPS (poly(2-acrylamido-2-methyl-1-propanesulfonic acid).

The “isoelectric point” or “pI” of a protein or antibody corresponds to a pH value at which the net charge of the protein or antibody is neutral. The pI can be determined by standard experimentation methods, for example by isoelectric focusing or by computational methods (“theoretical pI”). An example of a computational method is the free online standard tool “ExPASy” (http://web.expasy.org/compute_pi/), which calculates the pI based on the amino acid sequences of the protein or antibody. The theoretical pI of trastuzumab is 8.4 and the theoretical pI of pertuzumab is 8.7.

A “mobile phase” is the liquid or gas that flows through a chromatography system, moving the materials to be separated at different rates over the stationary phase. Preferably the mobile phase is liquid. In one example, the mobile phase can be the loading buffer (“mobile phase A”) or elution buffer (mobile phase B).

The “loading buffer” provides a condition to ensure that the target molecules interact effectively with the ligand of the ion exchange chromatography material and are retained by the affinity medium as all other molecules wash through the column.

The “elution buffer” is used to wash away unbound proteins at first and at a greater concentration it releases the charge variants and native antibodies from the ligand.

The term “main species antibody” or “native antibody” herein refers to the antibody amino acid sequence structure in a composition which is the quantitatively predominant antibody molecule in the composition. In terms of a fixed dose combination of two anti-HER2 antibodies, two main species antibodies are part of the composition. Thus, in one embodiment, the main species antibodies are an antibody that binds to extracellular subdomain II of HER2 and an antibody that binds to extracellular subdomain IV. In one embodiment, the main species antibodies of the FDC are pertuzumab and trastuzumab.

A “charge variant” “is a variant of the main species antibody, which has a different overall charge than the main species antibody. Examples of charge variants are acidic and basic variants.

An “acidic variant” is a variant of the main species antibody, which is more acidic than the main species antibody. An acidic variant has gained negative charge or lost positive charge relative to the main species antibody. Such acidic variants can be resolved using a separation methodology, such as ion exchange chromatography, that separates proteins according to charge. Acidic variants of a main species antibody elute earlier than the main peak upon separation by cation exchange chromatography. Acidic variants of pertuzumab and trastuzumab can be separated and quantified by the ion exchange chromatography method described herein. Examples of acidic pertuzumab variants are pertuzumab deamidated at the heavy chain asparagine at position 391 (HC-Asn-391), pertuzumab Fc sialic acid variant, and pertuzumab lysine glycation variant. Examples of acidic trastuzumab variants are trastuzumab deamidated at LC-Asn-30 and trastuzumab deamidated at HC-Asn-55.

A “basic variant” is a variant of the main species antibody, which is more basic than the main species antibody. A basic variant has gained positive charge or lost negative charge relative to the main species antibody. Such basic variants can be resolved using a separation methodology, such as ion exchange chromatography, that separates proteins according to charge. Basic variants of a main species antibody elute later than the main peak upon separation by cation exchange chromatography. Basic variants of pertuzumab and trastuzumab can be separated and quantified by the ion exchange chromatography method described herein.

The term “gradient” as used herein means a change of properties in the mobile phase during a chromatography sample run. In a “continuous gradient” one or more conditions of the mobile phase, for example the pH, the ionic strength, concentration of a salt, and/or the flow of the mobile phase is is changed, i.e. raised or lowered, continuously. The change can be linear or exponential or asymptotical. In a “step-wise gradient” one or more conditions, for example the pH, the ionic strength, concentration of a salt, and/or the flow of a chromatography, can be changed incrementally, i.e. stepwise, in contrast to a linear change.

The term “RP-UHPLC” means Reversed Phase Ultra High Performance Liquid Chromatography. The term RP-HPLC stands for Reversed Phase High Performance Liquid Chromatography. HPLC is used to separate compounds based on their polarities and interactions with the column's stationary phase. Reversed-phase chromatography is an elution procedure used in liquid chromatography in which the mobile phase is significantly more polar than the stationary phase.

A “RP-HPLC phenyl column” as used herein refer columns with hydrophobic phenyl groups present on the column packing material or resin (stationary phase). For example, a phenyl column exposes the material flowing through the column to unsubstituted phenyl groups. Phenyl columns contain for example short alkyl phenyl ligands covalently bound to the silica surface, or diphenyl phases. Some phenyl columns have phenyl group(s) with alkyl spacers between the phenyl group(s) and the silica surface. By increasing the length of the alkyl spacer, steric selectivity and aromatic selectivity can be enhanced. RP-HPLC phenyl columns differ by the number of aromatic groups (mono versus biphenyl), the length of the alkyl spacer between the silica surface and the phenyl group, the nature of the substituent groups on the bonded ligands (typically methyl or more sterically bulky isobutyl groups), the inclusion of an oxygen atom in the linker to activate the π electron system in the aromatic ring, and finally whether the silica stationary surface is endcapped or not. For example RP-HPLC phenyl columns can have the following groups: Ethyl phenyl with methyl side groups and an endcapped silica surface, Phenyl hexyl phase with extended (hexyl) ligand spacer methyl side groups, Ethyl phenyl ligand with steric protection (isobutyl) side groups, Hexyl biphenyl with methyl side groups, Biphenyl phase with methyl side groups, Oxygen activated phenyl ethyl phenyl phase with methyl side groups. HPLC columns with stationary phases modified with phenyl (e.g. single phenyl, biphenyl, diphenyl, phenyl hexyl, phenyl propyl) are readily available from most major column suppliers, for example: Acclaim Phenyl-1 (Dionex), Pursuit® XRs Diphenyl, Pinnacle® Biphenyl, Zorbax® Eclipse® Plus Hexyl Phenyl, Ascentis Phenyl, Agilent Zorbax RRHD 300-Diphenyl and Agilent AdvanceBio RP mAb Diphenyl.

The terms “cancer” and “cancerous” refer to or describe the physiological condition in mammals that is typically characterized by unregulated cell growth.

An “advanced” cancer is one which has spread outside the site or organ of origin, either by local invasion (“locally advanced”) or metastasis (“metastatic”). Accordingly, the term “advanced” cancer includes both locally advanced and metastatic disease.

“Metastatic” cancer refers to cancer which has spread from one part of the body (e.g. the breast) to another part of the body.

A “refractory” cancer is one which progresses even though an anti-tumor agent, such as a chemotherapy or biologic therapy, such as immunotherapy, is being administered to the cancer patient. An example of a refractory cancer is one which is platinum refractory.

A “recurrent” cancer is one which has regrown, either at the initial site or at a distant site, after a response to initial therapy, such as surgery.

A “locally recurrent” cancer is cancer that returns after treatment in the same place as a previously treated cancer.

A “non-resectable” or “unresectable” cancer is not able to be removed (resected) by surgery.

“Early-stage breast cancer” herein refers to breast cancer that has not spread beyond the breast or the axillary lymph nodes. Such cancer is generally treated with neoadjuvant or adjuvant therapy.

“Neoadjuvant therapy” or “neoadjuvant treatment” or “neoadjuvant administration” refers to systemic therapy given prior to surgery.

“Adjuvant therapy” or “adjuvant treatment” or “adjuvant administration” refers to systemic therapy given after surgery.

Herein, a “patient” or “subject” is a human patient. The patient may be a “cancer patient,” i.e. one who is suffering or at risk for suffering from one or more symptoms of cancer, in particular breast cancer.

A “patient population” refers to a group of cancer patients. Such populations can be used to demonstrate statistically significant efficacy and/or safety of a drug, such as pertuzumab and/or trastuzumab.

A “relapsed” patient is one who has signs or symptoms of cancer after remission. Optionally, the patient has relapsed after adjuvant or neoadjuvant therapy.

A cancer or biological sample which “displays HER expression, amplification, or activation” is one which, in a diagnostic test, expresses (including overexpresses) a HER receptor, has amplified HER gene, and/or otherwise demonstrates activation or phosphorylation of a HER receptor.

A cancer or biological sample which “displays HER activation” is one which, in a diagnostic test, demonstrates activation or phosphorylation of a HER receptor. Such activation can be determined directly (e.g. by measuring HER phosphorylation by ELISA) or indirectly (e.g. by gene expression profiling or by detecting HER heterodimers, as described herein).

A cancer cell with “HER receptor overexpression or amplification” is one which has significantly higher levels of a HER receptor protein or gene compared to a noncancerous cell of the same tissue type. Such overexpression may be caused by gene amplification or by increased transcription or translation. HER receptor overexpression or amplification may be determined in a diagnostic or prognostic assay by evaluating increased levels of the HER protein present on the surface of a cell (e.g. via an immunohistochemistry assay; IHC). Alternatively, or additionally, one may measure levels of HER-encoding nucleic acid in the cell, e.g. via in situ hybridization (ISH), including fluorescent in situ hybridization (FISH; see WO98/45479 published October, 1998) and chromogenic in situ hybridization (CISH; see, e.g. Tanner et al., Am. J. Pathol. 157(5): 1467-1472 (2000); Bella et al., J. Clin. Oncol. 26: (May 20 suppl; abstr 22147) (2008)), southern blotting, or polymerase chain reaction (PCR) techniques, such as quantitative real time PCR (qRT-PCR). One may also study HER receptor overexpression or amplification by measuring shed antigen (e.g., HER extracellular domain) in a biological fluid such as serum (see, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al. J. Immunol. Methods 132: 73-80 (1990)). Aside from the above assays, various in vivo assays are available to the skilled practitioner. For example, one may expose cells within the body of the patient to an antibody which is optionally labeled with a detectable label, e.g. a radioactive in situ for radioactivity or by analyzing a biopsy taken from a patient previously exposed to the antibody.

A “HER2-positive” cancer comprises cancer cells which have higher than normal levels of HER2. Optionally, HER2-positive cancer has an immunohistochemistry (IHC) score of 2+ or 3+ and/or is in situ hybridization (ISH), fluorescent in situ hybridization (FISH) or chromogenic in situ hybridization (CISH) positive, e.g. has an ISH/FISH/CISH amplification ratio of ≥2.0.

A “HER2-mutated” cancer comprises cancer cells with a HER2-activating mutation, including kinase domain mutations, which can, for example, be identified by next generation sequencing (NGS) or real-time polymerase chain reaction (RT-PCR). “HER2-mutated” cancer specifically includes cancer characterized by insertions in exon 20 of HER2, deletions around amino acid residues 755-759 of HER2, any of the mutations G309A, G309E, S310F, D769H, D769Y, V777L, P780-Y781insGSP, V842I, R896C (Bose et al., Cancer Discov 2013; 3:1-14), as well as previously reported identical non-synonymous putative activating mutations (or indels) in COSMIC database found in two or more unique specimens. For further details see, e.g. Stephens et al., Nature 2004; 431:525-6; Shigematsu et al., Cancer Res 2005; 65:1642-6; Buttitta et al., Int J Cancer 2006; 119:2586-91; Li et al., Oncogene 2008; 27:4702-11; Sequist et al., J Clin Oncol 2010; 28:3076-83; Arcila et al., Clin Cancer Res 2012; 18:4910-8; Greulich et al., Proc Natl Acad Sci USA 2012; 109:14476-81; and Herter-Sprie et al., Front Oncol 2013; 3:1-10.

Herein, an “anti-tumor agent” refers to a drug used to treat cancer. Non-limiting examples of anti-tumor agents herein include chemotherapy agents, HER dimerization inhibitors, HER antibodies, antibodies directed against tumor associated antigens, anti-hormonal compounds, cytokines, EGFR-targeted drugs, anti-angiogenic agents, tyrosine kinase inhibitors, growth inhibitory agents and antibodies, cytotoxic agents, antibodies that induce apoptosis, COX inhibitors, farnesyl transferase inhibitors, antibodies that binds oncofetal protein CA 125, HER2 vaccines, Raf or ras inhibitors, liposomal doxorubicin, topotecan, taxane, dual tyrosine kinase inhibitors, TLK286, EMD-7200, pertuzumab, trastuzumab, erlotinib, and bevacizumab.

“Treatment” refers to both therapeutic treatment and prophylactic or preventative measures. Those in need of treatment include those already with cancer as well as those in which cancer is to be prevented. Hence, the patient to be treated herein may have been diagnosed as having cancer or may be predisposed or susceptible to cancer.

The term “effective amount” refers to an amount of a drug effective to treat cancer in the patient. The effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST, or CA-125 changes), result in an objective response (including a partial response, PR, or complete response, CR), increase overall survival time, and/or improve one or more symptoms of cancer (e.g. as assessed by FOSI).

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

A “chemotherapy” is use of a chemical compound useful in the treatment of cancer. Examples of chemotherapeutic agents, used in chemotherapy, include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; TLK 286 (TELCYTA™); acetogenins (especially bullatacin and bullatacinone); delta-9-tetrahydrocannabinol (dronabinol, MARINOL®);

beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; bisphosphonates, such as clodronate; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)) and anthracyclines such as annamycin, AD 32, alcarubicin, daunorubicin, doxorubicin, dexrazoxane, DX-52-1, epirubicin, GPX-100, idarubicin, valrubicin, KRN5500, menogaril, dynemicin, including dynemicin A, an esperamicin, neocarzinostatin chromophore and related chromoprotein enediyne antibiotic chromophores, aclacinomy sins, actinomycin, authramycin, azaserine, bleomycins, cactinomycin, carabicin, carminomycin, carzinophilin, chromomycinis, dactinomycin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin, liposomal doxorubicin, and deoxydoxorubicin), esorubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; folic acid analogues such as denopterin, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenisher such as folinic acid (leucovorin); aceglatone; anti-folate anti-neoplastic agents such as ALIMTA®, LY231514 pemetrexed, dihydrofolate reductase inhibitors such as methotrexate, anti-metabolites such as 5-fluorouracil (5-FU) and its prodrugs such as UFT, S-1 and capecitabine, and thymidylate synthase inhibitors and glycinamide ribonucleotide formyltransferase inhibitors such as raltitrexed (TOMUDEX^{RM}, TDX); inhibitors of dihydropyrimidine dehydrogenase such as eniluracil; aldophosphamide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK7 polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); cyclophosphamide; thiotepa; taxanes; chloranbucil; gemcitabine (GEMZAR®); 6-thioguanine; mercaptopurine; platinum; platinum analogs or platinum-based analogs such as cisplatin, oxaliplatin and carboplatin; vinblastine (VELBAN®); etoposide (VP-16); ifosfamide; mitoxantrone; vincristine (ONCOVIN®); vinca alkaloid; vinorelbine (NAVELBINE®); novantrone; edatrexate; daunomycin; aminopterin; xeloda; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

A “taxane” is a chemotherapy which inhibits mitosis and interferes with microtubules. Examples of taxanes include Paclitaxel (TAXOL®; Bristol-Myers Squibb Oncology, Princeton, N.J.); cremophor-free, albumin-engineered nanoparticle formulation of paclitaxel or nab-paclitaxel (ABRAXANE™; American Pharmaceutical Partners, Schaumberg, Illinois); and Docetaxel (TAXOTERE®; Rhone-Poulenc Rorer, Antony, France).

An “anthacycline” is a type of antibiotic that comes from the fungus Streptococcus peucetius, examples include: Daunorubicin, Doxorubicin, Epirubicin, and any other anthracycline chemotherapeutic agents, including those listed before.

“Anthracycline-based chemotherapy” refers to a chemotherapy regimen that consists of or includes one or more anthracycline. Examples include, without limitation, 5-FU, epirubicin, and cyclophosphamide (FEC); 5-FU, doxorubicin, and cyclophosphamide (FAC); doxorubicin and cyclophosphamide (AC); epirubicin and cyclophosphamide (EC); dose-dense doxorubicin and cyclophosphamide (ddAC), and the like.

For the purposes herein, “carboplatin-based chemotherapy” refers to a chemotherapy regimen that consists of or includes one or more Carboplatins. An example is TCH (Docetaxel/TAXOL®, Carboplatin, and trastuzumab/HERCEPTIN®).

An “aromatase inhibitor” inhibits the enzyme aromatase, which regulates estrogen production in the adrenal glands. Examples of aromatase inhibitors include: 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In one embodiment, the aromatase inhibitor herein is letrozole or anastrozole.

An “antimetabolite chemotherapy” is use of an agent which is structurally similar to a metabolite, but cannot be used by the body in a productive manner Many antimetabolite chemotherapy interferes with the production of the nucleic acids, RNA and DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOME®), azocytosine, deoxycytosine, pyridmidene, fludarabine (FLUDARA®), cladrabine, 2-deoxy-D-glucose etc.

By “chemotherapy-resistant” cancer is meant that the cancer patient has progressed while receiving a chemotherapy regimen (i.e. the patient is “chemotherapy refractory”), or the patient has progressed within 12 months (for instance, within 6 months) after completing a chemotherapy regimen.

The term “platin” is used herein to refer to platinum based chemotherapy, including, without limitation, cisplatin, carboplatin, and oxaliplatin.

The term “fluoropyrimidine” is used herein to refer to an antimetabolite chemotherapy, including, without limitation, capecitabine, floxuridine, and fluorouracil (5-FU).

A “fixed” or “flat” dose of a therapeutic agent herein refers to a dose that is administered to a human patient without regard for the weight (WT) or body surface area (BSA) of the patient. The fixed or flat dose is therefore not provided as a mg/kg dose or a mg/m² dose, but rather as an absolute amount of the therapeutic agent.

### II. Assays

Co-formulation of therapeutic monoclonal antibodies (mAbs) to a fixed dose combination (FDC) increases the complexity of the drug product, and creates challenges for characterization and control of product quality. This challenge is exacerbated when the coformulated antibodies have similar physicochemical properties, like similar isoelectric points, sequence similarities, and no significant difference in size. Moreover, each of the coformulated antibodies can exhibit heterogeneities in size, charge, and post-translational modifications during manufacturing. For these reasons, interactions between the mAbs in fixed dose combination need to be characterized and understood. Herein described are analytical methods to determine critical quality attributes (CQAs) of a fixed dose combination of two anti-HER2 antibodies.

In one aspect, these assays are suitable to analyze a fixed dose combination of the two anti-HER2 antibodies trastuzumab and pertuzumab. Trastuzumab and pertuzumab have more than 93% sequence identity, differ only by 30 Da and both have a molecular weight of approx. 148 kDa. Furthermore, both antibodies have very similar isoelectric points, bind to the same target (HER2) and have a synergistic effect in vivo. Due to these structural and functional similarities, most of the usual known analytical methods cannot be applied to this co-formulation. In addition, the assays developed for the testing strategy took into account that the trastuzumab pertuzumab fixed dose combination is provided in two different dosages, i.e. loading dose and maintenance dose, which differ in the ratio of pertuzumab SC and trastuzumab SC drug substances.

(i) Potency Assays

Potency is a CQA that is included in the control system for release and stability testing of biotherapeutics, including therapeutic monoclonal antibodies. Potency monitors the cumulative impact of product quality attributes on bioactivity, which can potentially impact safety and efficacy; namely, higher potency can pose safety concerns, whereas lower potency can raise considerations for efficacy. Ideally, the potency assay will represent the product's mechanism of action (i.e., relevant therapeutic activity or intended biological effect). According to the US Food and Drug Administration's (FDA's) “Guidance for Industry on Potency Tests for Cellular and Gene Therapy Products”, the traditional approach for assessing the potency of biological products is to develop a quantitative biological assay (bioassay) that measures the activity of the product related to its specific ability to effect a given result. Bioassays can provide a measure of potency by evaluating a product's active ingredient(s) within a living biological system. Bioassays can include in vivo animal studies, in vitro organ, tissue or cell culture systems, or any combination of these. A widely used example of a bioassay for determining or quantifying potency is a cell-based assay. Two distinct cell-based assays, designed to measure cell growth inhibition specifically for pertuzumab or trastuzumab (anti-proliferation assays) were assessed for their suitability to control the biological activity of the fixed dose combination of pertuzumab trastuzumab. This assessment demonstrated that the assays are not suitable for the fixed dose combination because of limitations that prevent the control of relevant changes in product quality of the individual antibodies, when combined in the co-formulation. Due to the nature of the co-formulation of two antibodies that bind to the same receptor and inhibit similar signaling pathways, no alternative HER2-expressing cell line would be able to overcome these limitations.

For trastuzumab and pertuzumab, which bind to the same receptor and act on similar signaling pathways in the target cells, effects on downstream signaling, gene expression, and proliferation of HER2-expressing target cells are mediated by their binding activity to the respective epitopes on HER2. Therefore, potential molecular changes of the antibodies that affect their potency to inhibit HER2-driven cell growth can be observed at the binding level. This hypothesis has been assessed in a comparative study with selected product variants (charge and size variants and CDR affinity mutants), as shown in the examples herein. The study confirmed that the difference in binding as detected by the binding assays provided therein reflect the changes observed in the anti-proliferation activity for most of the product variants tested, except for size variants. Given the interference in the anti-proliferation assays and the ability of the binding assays provided therein to detect single-antibody quality changes affecting potency, the new binding assays provided therein are considered the best possible assays to control relevant changes in product quality affecting target binding and HER2 signaling.

In one embodiment the pertuzumab trastuzumab FDC drug product is tested by binding assays that specifically measure HER2 binding to pertuzumab or trastuzumab to determine potency. Trastuzumab and pertuzumab both target HER2, but they bind to distinct and non-overlapping epitopes on the HER2 extracellular domain (ECD): trastuzumab recognizes subdomain IV, the juxtamembrane region, while pertuzumab recognizes subdomain II, the dimerization region (Rocca A, Andreis D, Fedeli A, et al. Pharmacokinetics, pharmacodynamics and clinical efficacy of pertuzumab in breast cancer therapy. Expert Opin Drug Metab Toxicol 2015; 11:1647-63). Binding of trastuzumab to the HER2 subdomain IV inhibits ligand-independent HER2 signaling by blocking its homodimerization (Junttila T T, Akita R W, Parsons K, et al. Ligand-independent HER2/HER3/PI3K complex is disrupted by trastuzumab and is effectively inhibited by the PI3K inhibitor GDC-0941. Cancer Cell 2009; 15:429-40), and prevents the proteolytic cleavage of its ECD, thereby prohibiting subsequent constitutive activation of associated intracellular signaling pathways (Molina M A, Codony-Servat J, Albanell J, et al. Trastuzumab (Herceptin), a humanized anti-HER2 receptor monoclonal antibody, inhibits basal and activated HER2 ectodomain cleavage in breast cancer cells. Cancer Research 2001; 61:4744-9). As a result, trastuzumab inhibits the proliferation of human tumor cells that overexpress HER2, as has been shown in both in vitro assays and animals. Binding of pertuzumab to the HER2 subdomain II blocks ligand-dependent heterodimerization of HER2 with other HER family members, including EGFR, HER3, and HER4 (Franklin M C, Carey K D, Vajdos F F, et al. Insights into ErbB signaling from the structure of the ErbB2 pertuzumab complex. Cancer Cell 2004; 5:317-28; Adams C W, Allison D E, Flagella K, et al. Humanization of a recombinant monoclonal antibody to produce a therapeutic HER dimerization inhibitor, pertuzumab. Cancer Immunol Immunother 2006; 55:717-27; Diermeier-Daucher S, Hasmann M, Brockhoff G. Flow cytometric FRET analysis of erbB receptor interaction on a cell by cell basis. Ann NY Acad Sci 2008; 1130:280-6). As a result, pertuzumab inhibits ligand-initiated intracellular signaling, inducing cell growth arrest and apoptosis of human tumor cells that overexpress HER2. Pertuzumab and trastuzumab bind to these distinct and non-overlapping epitopes on the HER2 ECD without competing with each other, and they have complementary mechanisms for disrupting HER2 signaling. This results in augmented anti-proliferative activity in vitro and in vivo when pertuzumab and trastuzumab are administered in combination (Scheuer W, Friess T, Burtscher H, et al. Strongly enhanced antitumor activity of trastuzumab and pertuzumab combination treatment on HER2-positive human xenograft tumor models. Cancer Res 2009; 69:9330-6). In one embodiment the anti-proliferative activity and HER2 signaling of the FDC drug product is determined using two distinct HER2-binding assays, which ensure control of the quality of each of the two antibodies in the pertuzumab trastuzumab FDC drug product.

In one embodiment a binding assay for a fixed dose combination (FDC) of two anti-HER2 antibodies is provided, comprising
a. contacting the FDC with a capture reagent, wherein the capture reagent is a modified HER2 ECD.
b. contacting the sample with a detectable antibody.
c. quantifying the level of antibody bound to the capture reagent using a detection means for the detectable antibody.

The fixed dose combination of two anti-HER2 antibodies is contacted and incubated with the capture reagent so that the capture reagent captures or binds to one of the anti-HER2 antibodies of interest so that it can be detected in a detection step. The capture reagent is a modified HER2 ECD comprising one or more recombinant HER2 ECD subdomains. In one embodiment the modified HER2 ECD is a genetically engineered protein or peptide that comprises one or more recombinant HER2 ECD subdomains. In one embodiment the HER2 ECD is modified such that one of the anti-HER2 antibodies to be assessed in the FDC can bind while the second anti-HER2 antibody in the FDC will not bind to it. This is achieved by either omitting the HER2 ECD subdomain to which the second anti-HER2 antibody binds to or by replacing it by a structurally close subdomain that is not bound to by either of the anti-HER2 antibodies. A structurally close subdomain can be any subdomain that when included in the modified HER2 ECD does not interrupt the three-dimensional conformation of the modified HER2 ECD. Examples of structurally close subdomains are corresponding subdomains of EGFR, HER3 or HER4. Preferably the modified HER2 ECD has a three-dimensional conformation mimicking the native HER2 ECD as closely as possible. The subdomains can be full-length or shortened by a few amino acids at the N or C-terminus. It has been found by the inventors of the present invention that the integrity of the three-dimensional structure of the HER2 ECD is retained or improved when using one or more recombinant HER2 ECD subdomains that are shortened by about 4 to 5 amino acids at the C-terminus.

In one embodiment the modified HER2 ECD is fused to a peptide or protein to facilitate immobilizing the capture reagent to a solid substrate. Examples of suitable peptides or proteins are biotin, bovine serum albumin (BSA) and Fc domains. In one modified HER2 extracellular domain is fused to a Fc domain. In one embodiment said Fc domain is from a species different from the species of the Fc domain of the anti-HER2 antibody to be analysed. For example, if the anti-HER2 antibody to be analyzed comprises a human Fc domain, the capture reagent should comprise a non-human Fc domain, e.g. murine, porcupine, rat, rabbit and so forth. In one embodiment the Fc domain of the recombinant HER2 ECD subdomain is a murine Fc domain. In one embodiment said Fc domain comprises SEQ ID NO. 35.

In a next step, the sample comprising the capture reagent and the captured anti-HER2 antibody is incubated with a detectable antibody. The detectable antibody, when contacted with any of the bound anti-HER2 antibody of interest, binds to the antibody of interest. In a next step, a detection means is used to detect the label on the detectable antibody and hence the presence or amount of anti-HER2 antibody of interest present in the FDC.

In one embodiment the fixed dose combination comprises an antibody binding to HER2 extracellular subdomain II and an antibody binding to HER2 extracellular subdomain IV. In one embodiment the antibody binding to HER2 extracellular subdomain II is pertuzumab. In one embodiment the antibody binding to HER2 extracellular subdomain IV is trastuzumab. In one embodiment the fixed dose combination comprises pertuzumab and trastuzumab. In one embodiment the fixed dose combination additionally comprises hyaluronidase. In one such embodiment the hyaluronidase is a recombinant human hyaluronidase. In one preferred embodiment said hyaluronidase is rHUPH20. The pertuzumab trastuzumab FDC drug product is provided in two different dosages, i.e. a loading dose (LD) and a maintenance dose (MD). The LD and MD have the same total protein content and differ in the ratio of pertuzumab SC and trastuzumab SC drug substances. In one embodiment the binding assay is used to analyze a LD of a pertuzumab trastuzumab FDC. In one embodiment the binding assay is used to analyze a pertuzumab trastuzumab FDC comprising 40 mg/mL trastuzumab and 80 mg/mL pertuzumab. In one embodiment said pertuzumab trastuzumab FDC additionally comprises rHuPH20 at 2000 U/mL. In one embodiment the binding assay is used to analyze a MD of a pertuzumab trastuzumab FDC. In one embodiment the binding assay is used to analyze a pertuzumab trastuzumab FDC comprising 60 mg/mL and trastuzumab, 60 mg/mL pertuzumab. In one embodiment said pertuzumab trastuzumab FDC additionally comprises rHuPH20 at 2000 U/mL.

In one embodiment the binding of pertuzumab and trastuzumab are determined in two separate binding assays.

The pertuzumab binding assay determines specific bioactivity as the ability of pertuzumab to specifically bind to its epitope of the recombinant HER2 capture reagent. In one embodiment the binding of Pertuzumab is quantified. In one such embodiment the capture reagent comprises HER2 extracellular subdomain II or parts thereof. In one embodiment the capture reagent comprises human HER2 extracellular subdomain II In one embodiment the capture reagent comprises SEQ ID NO. 23 or sequence ID No: 2.

In one embodiment the modified HER2 ECD comprises HER2 ECD subdomains I, II and III or parts thereof. In one embodiment the modified HER2 ECD comprises human HER2 ECD subdomains I, II and III or parts thereof. In one embodiment the modified HER2 ECD does not comprise subdomain IV. It has been found by the present inventors that a modified HER2 ECD can be produced with a three-dimensional conformation resembling the native HER2 ECD, when including a recombinant subdomain III which has been truncated at the C-terminus. In one such embodiment the modified HER2 ECD comprises SEQ ID NO: 1, SEQ ID NO: 2 and SEQ ID NO:34. In one embodiment the modified HER2 ECD comprises SEQ ID NO. 24. In one embodiment the modified HER2 ECD has at least 99%, 98%, 97%, 96%, 95%, or 90% sequence identity to SEQ ID NO. 24.

In one embodiment, the recombinant HER2 extracellular subdomains I, II, III are fused to a Fc domain. In one embodiment said Fc domain is a murine, rat, rabbit or porcupine Fc domain. In any of the above embodiments the capture reagent for assessing binding of Pertuzumab does not comprise a HER2 subdomain IV. In one embodiment the capture reagent comprises SEQ ID NO: 25, SEQ ID NO: 26 or SEQ ID NO: 27. In one embodiment the modified HER2 ECD has at least 99%, 98%, 97%, 96%, 95%, or 90% sequence identity to SEQ ID NO. 25. In one embodiment the modified HER2 ECD has at least 99%, 98%, 97%, 96%, 95%, or 90% sequence identity to SEQ ID NO. 26. In one embodiment the modified HER2 ECD has at least 99%, 98%, 97%, 96%, 95%, or 90% sequence identity to SEQ ID NO. 27.

In one embodiment the binding of trastuzumab is quantified. In one such embodiment the capture reagent comprises recombinant HER2 extracellular subdomain IV or parts thereof. In one such embodiment the capture reagent comprises human recombinant HER2 extracellular subdomain IV. In one embodiment the capture reagent comprises SEQ ID NO. 28 or sequence ID No: 4. In one embodiment the capture reagent comprises recombinant HER2 extracellular subdomains I, III and IV. In one embodiment the capture reagent comprises human HER2 extracellular subdomains I, III and IV. In one embodiment the capture reagent comprises recombinant HER2 extracellular subdomains I, III and IV and subdomain II of EGFR. In one embodiment the capture reagent comprises recombinant human HER2 extracellular subdomains I, III and IV and recombinant human subdomain II of EGFR. It has been found by the present inventors that a modified HER2 ECD can be produced with a three-dimensional conformation resembling the native HER2 ECD, when including a recombinant HER2 extracellular subdomain I and a recombinant HER2 extracellular subdomain IV, which both have been truncated at the C-terminus. In one embodiment the modified ECD comprises SEQ ID NO: 33, SEQ ID NO: 3 and SEQ ID NO: 28. In one embodiment the modified ECD comprises SEQ ID NO: 33, SEQ ID NO: 36, SEQ ID NO: 3 and SEQ ID NO: 28.

In one embodiment the modified HER2 ECD comprises SEQ ID NO. 29. In one embodiment the modified HER2 ECD has at least 99%, 98%, 97%, 96%, 95%, or 90% sequence identity to SEQ ID NO. 29.

In one embodiment the recombinant HER2 extracellular subdomains I, III and IV and subdomain II of EGFR are fused to a Fc domain. In one embodiment said Fc domain is a murine, rat, rabbit or porcupine Fc domain. In any of the above embodiments the capture reagent for assessing binding of trastuzumab does not comprise a HER2 subdomain II In one embodiment the capture reagent comprises SEQ ID NO: 30, SEQ ID NO: 31 or SEQ ID NO: 32. In one embodiment the modified HER2 ECD has at least 99%, 98%, 97%, 96%, 95%, or 90% sequence identity to SEQ ID NO. 30. In one embodiment the modified HER2 ECD has at least 99%, 98%, 97%, 96%, 95%, or 90% sequence identity to SEQ ID NO. 31. In one embodiment the modified HER2 ECD has at least 99%, 98%, 97%, 96%, 95%, or 90% sequence identity to SEQ ID NO. 32.

In any of the above embodiments, the detectable antibody comprises a label which allows for its detection by various means. These labels include moieties that may be detected directly, such as fluorochrome, chemiluminescent, and radioactive labels, as well as moieties, such as enzymes, that must be reacted or derivatized to be detected. Examples of such labels include the radioisotopes 32P, 14C, 1251, 3H, and 1311, fluorophores such as rare-earth chelates or fluorescein and its derivatives, rhodamine and its derivatives, ruthenium, dansyl, umbelliferone, luciferases, e.g., firefly luciferase and bacterial luciferase (U.S. Pat. No. 4,737,456), luciferin, 2,3-dihydrophthalazinediones, HRP, alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin (detectable by, e.g., avidin, streptavidin, streptavidin-HRP, and streptavidin-β-galactosidase with MUG), spin labels, bacteriophage labels, stable free radicals, and the like.

The preferred label of the detectable antibody is Horse Radish Peroxidase (HRP). The substrates commonly used with HRP fall into different categories including chromogenic (e g aminoethyl carbazole (AEC), 3, 3′-diaminobenzidine tetrahydrochloride (DAB), chloronaphthol combined with diaminobenzidine (CN/DAB), Tetramethyl Benzidine (TMB), o-phenylenediamine dihydrochloride (OPD), 2,2′-Azinobis [3-ethylbenzothiazoline-6-sulfonic acid]-diammonium salt (ABTS)), fluorogenic (e.g. ADHP) and chemiluminescent (e g enhanced chemiluminescence (ECL)) substrates depending on whether they produce a colored, fluorimetric or luminescent derivative respectively. A preferred substrate is ABTS.

In one embodiment the detectable antibody targets the F(ab′)2 portion of human IgG. In one embodiment the detectable antibody targets the F(ab′)2 portion of the anti-HER2 antibody.

In one embodiment, the binding assay is an enzyme-linked immunoabsorbent assay (ELISA). In an ELISA the capture reagent is attached to a solid substrate. The solid phase used for immobilization may be any inert support or carrier that is essentially water insoluble and useful in immunometric assays, including supports in the form of, e.g., surfaces, particles, porous matrices, etc. Examples of commonly used supports include small sheets, SEPHADEX® gels, polyvinyl chloride, plastic beads, and assay plates or test tubes manufactured from polyethylene, polypropylene, polystyrene, and the like, including 96-well microtiter plates, as well as particulate materials such as filter paper, agarose, cross-linked dextran, and other polysaccharides. Alternatively, reactive water-insoluble matrices such as cyanogens-bromide-activated carbohydrates and the reactive substrates described in U.S. Pat. Nos. 3,969,287; 3,691,016; 4,195,128; 4,247,642; 4,229,537; and 4,330,440 are suitably employed for capture-reagent immobilization. In a preferred embodiment, the immobilized capture reagents are coated on a microtiter plate, and in particular a preferred solid phase used is a multi-well microtiter plate that can be used to analyze several samples at one time. Preferred microtiter plates are plates with a highly charged polystyrene surface with high affinity for molecules with polar or hydrophilic groups, which have a high binding capacity for proteins. The most preferred is a MICROTEST® or MAXISORP® 96-well ELISA plate such as that sold as NUNC MAXISORB® or IMMULON®.

The 96-well plates are preferably coated with the capture reagent for at least 30 minutes, 40 minutes, 50 minutes, 60 minutes, about 20 to 80 minutes, or about 30 to 60 minutes. The 96-well plates are preferably coated with the capture reagent at temperatures of about 4-20° C., more preferably at about 2-8° C. The plates may be stacked and coated in advance of the assay itself, and then the assay can be carried out simultaneously on several samples in a manual, semi-automatic, or automatic fashion, such as by using robotics.

The amount of capture reagents employed is sufficiently large to give a good signal, but not in molar excess compared to the maximum expected level of antibody of interest in the sample. In one embodiment the coat reagent concentration is about 0.5 μg/mL-5 μg/mL, preferably about 1 μg/mL-1.5 μg/mL.

The coated plates are then typically treated with a blocking agent that binds non-specifically to and saturates the binding sites to prevent unwanted binding of the free ligand to the excess sites on the wells of the plate. Examples of appropriate blocking agents for this purpose include, e.g., gelatin, bovine serum albumin (BSA), egg albumin, casein, and non-fat milk. The blocking treatment typically takes place under conditions of ambient temperatures for about 1-4 hours, about 1 to 3 hours, preferably about 1 to 1.5 hours.

After coating and blocking, the standard or the FDC sample to be analyzed, is added in standard dilutions to the coated plates. In one embodiment, increasing concentrations of pertuzumab trastuzumab FDC (standard, product control and samples to be analyzed) are added to the coated plates.

The conditions for incubation of the FDC sample and immobilized capture reagent are selected to maximize sensitivity of the assay and to minimize dissociation, and to ensure that the anti-HER2 antibody to be assessed in the FDC sample binds to the immobilized capture reagent. Preferably, the incubation is accomplished at fairly constant temperatures, ranging from about 0° C. to about 40° C., preferably at or about room temperature. The time for incubation is generally no greater than about hours. Preferably, the incubation time is from about 0.5 to 3 hours, and more preferably about 1 to 1.5 hours at or about room temperature to maximize binding of the anti-HER2 antibody to be assessed in the FDC sample to the capture reagents.

The immobilized capture reagents with any bound anti-HER2 antibody are contacted with detectable antibody, preferably at a temperature of about 20-40° C., more preferably at room temperature, with the exact temperature and time for contacting the two being dependent primarily on the detection means employed.

In another embodiment the binding assay is an electrochemiluminescence (ECL).

In one embodiment the binding assay is used for analyzing the potency of one of the anti-HER2 antibodies. Thus, in one embodiment the binding assay additionally comprises step
d. correlating the level of antibody bound to the capture reagent with the biological activity of said antibody.

In one embodiment a dose-response curve generated for the samples is compared to a dose-response curve of a standard. In one embodiment the potency of the standard is quantified by separately correlating the results obtained in the binding assay with the biological activity of the isolated antibodies in a cell-based assay.

In one embodiment, non-linear four-parameter dose-response curves generated for the sample and the standard are compared. Once the similarity criteria between the standard and the sample dose-response curve are assessed, the relative potency of a sample is calculated based on the concentration shift between standard and sample dose-response curve fit and using four-parameter parallel line analysis.

In one embodiment the binding assay is for batch release of a fixed dose combination of pertuzumab and trastuzumab. In one embodiment the binding assay is for determining shelf-life of a fixed dose combination of pertuzumab and trastuzumab. In one such embodiment, the pertuzumab trastuzumab FDC is analyzed with the binding assays of the above embodiments at several points in time during storage.

(ii) Analysis of Charge Variants

In one embodiment a method for evaluating a fixed dose composition comprising pertuzumab, trastuzumab is provided, said method comprising assessing the amount of charge variants of pertuzumab and trastuzumab in the composition. In one embodiment said fixed dose combination additionally comprises hyaluronidase. In one embodiment said method is an ion exchange chromatography. Ion-exchange chromatography (IEX) is widely used for the detailed characterization of therapeutic proteins and can be considered as a reference and powerful technique for the qualitative and quantitative evaluation of charge heterogeneity. Ion-Exchange High Performance Liquid Chromatography (IE-HPLC, IEC) separates molecules in solution according to their charge heterogeneity. Separation is caused by the reversible adsorption of charged solute molecules onto ion-exchange groups of opposite charge immobilized in the column packing material. The adsorption of the molecules to the solid support is driven by the ionic interaction between the two moieties. The strength of the interaction is determined by the number and location of the charges on the molecule and on the stationary phase. IEX is typically a release method where specifications are set around the distribution of each acidic, main, and basic species specifically for mAbs. These charged species are considered product related impurities that may impact potency. Moreover, it is one of the few methods that can characterize the protein in its native confirmation as no denaturants are added. IEX may also be used as an identity method for certain biologics and is a routine test for stability and shelf-life justification.

Analyzing the distribution of charge variants of a Fixed Dose Combination of two anti-HER2 antibodies with very similar isoelectric points, like trastuzumab and pertuzumab, requires a specific ion-exchange chromatography protocol to allow for proper segregation of all relevant species.

In one embodiment a method for evaluating a fixed dose composition comprising pertuzumab, trastuzumab is provided, said method comprising assessing the amount of charge variants of pertuzumab and trastuzumab in the composition. In one embodiment said fixed dose combination additionally comprises hyaluronidase. In one embodiment said method is an ion exchange chromatography. In one specific embodiment said method is a cation exchange chromatography. In cation-exchange chromatography, as applied for pertuzumab/trastuzumab Fixed-Dose Combination (FDC), positively charged molecules are retained on a negatively charged stationary phase. Acidic species elute at lower retention times than basic species.

After equilibration of the column and sample application, the anti-HER2 antibodies pertuzumab, trastuzumab of the FDC are adsorbed to the column ligand. The column is then washed to remove unadsorbed proteins and elution is performed by changing the ionic strength of the mobile phase while keeping the pH within a predefined range. In one embodiment the pH is kept at a constant value.

The ionic strength is changed by applying a gradient of increasing salt concentration, the gradient being either a step gradient or a continuous gradient. The inventors of the present invention found that for analyzing charge variants of a FDC of the two anti-HER2 antibodies pertuzumab, trastuzumab, the pH range of the loading buffer (mobile phase A) and elution buffer (mobile phase B) is critical. The best separation of charge variants is obtained with a predefined pH range of the loading buffer (mobile phase A) of pH 7.5-7.65 and a predefined pH range of the elution buffer (mobile phase B) of pH 7.5-7.7. In one embodiment the pH is kept at a constant value. In one embodiment said constant pH value of the loading buffer is 7.5, 7.55, 7.6 or 7.65. In one embodiment said constant pH value of the elution buffer is 7.5, 7.55, 7.6, 7.65 or 7.7.

After elution, the column is then re-equilibrated with the loading buffer (mobile phase A).

In one embodiment the pertuzumab trastuzumab Fixed Dose combination is contacted with a cation exchange material and the charge variants and native antibodies are eluted with a salt gradient while keeping the pH of the mobile phase within a predefined range. In one embodiment the salt gradient is a continuous salt gradient. In one embodiment the pH of the mobile phase of the loading buffer (mobile phase A) is between pH 7.5 and pH 7.65. In one embodiment the pH of the mobile phase of the eluting buffer (mobile phase B) is between pH 7.5 and pH 7.7.

In one embodiment the salt gradient is a sodium chloride gradient. In one embodiment the salt gradient is a sodium chloride gradient and the pH of the mobile phase of the eluting buffer (mobile phase B) is between pH 7.5 and pH 7.7.

In one embodiment a method for evaluating a fixed dose composition comprising pertuzumab, and trastuzumab is provided, said method comprising
a. Binding the antibodies to a ion exchange material using a loading buffer, wherein the pH of the loading buffer is between about pH 7.5 and about pH 7.65
b. Eluting the antibodies with an elution buffer, wherein the pH of the elution buffer is between about pH 7.5 and about pH 7.7

In one embodiment the elution of step b is performed using a salt gradient. In one embodiment said salt gradient is a continuous salt gradient. In one embodiment the salt gradient is a sodium (Na+) gradient. Thus in one embodiment said elution buffer comprises sodium. In one embodiment the elution buffer comprises sodium ions (Na+). In one embodiment the sodium gradient is a sodium chloride (NaCl) gradient. In one embodiment said elution buffer comprises NaCl. Suitable buffers for the loading and elution buffer are MES (2-ethanesulfonic acid), ACES (N-(2-Acetamido)-2-aminoethanesulfonic acid), HEPES (4-(2-hydroxyethyl)-1-piperazineethanesulfonic acid), phosphate buffer, MOPS (3-(N-morpholino)propanesulfonic acid), TAPS ([tris(hydroxymethyl)methylamino]propanesulfonic acid), CAPSO (N-cyclohexyl-2-hydroxyl-3-aminopropanesulfonic acid), Tris (tris(hydroxymethyl)aminomethane), PIPES (piperazine-N,N′-bis(2-ethanesulfonic acid)), TPP (Tris, phosphate, piperazine). Preferred buffers are ACES and HEPES.

In one embodiment the sodium chloride concentration of the elution buffer (mobile phase B) is about 180-220 mM NaCl, about 200 mM NaCl, about 180 mM NaCl, about 190 mM NaCl, about 210 mM NaCl or about 220 mM NaCl.

In one embodiment said ion exchange material is an cation exchange material. When further optimizing the method of the invention, the inventors found that separation of charge variants is improved when using strong cation exchange column material. In a preferred embodiment the method is performed using a non-porous SCX column with sulfonate groups, using Na+ as counterion for elution. Thus in one embodiment the cation exchange material has sulfonate groups. In one such embodiment the cation exchange material is a strong cation exchanger (SCX) column with sulfonate groups and the elution buffer comprises sodium. In one such embodiment the elution buffer comprises sodium ions. In one embodiment said SCX column is non-porous. Preferred cation exchange columns useful therein are: YMC Bio Pro SP-F column, MabPac SCX-10, Waters BioResolve SCX mAb, Sepax Proteomix SCX-NP1.7 or Agilent Bio SCX non-porous.

In one embodiment step a and b of the method above are performed at a temperature of 32° C. to 40° C. or at about 36° C.

In one embodiment the ion exchange chromatography is performed with loading a total protein amount of about 50 μg to 149 μg, or about 51 μg to 153 μg. In one embodiment the ion exchange chromatography is performed with loading a total protein amount of about 50 μg to 149 μg of a Loading Dose of a pertuzumab trastuzmab FDC. In one embodiment the ion exchange chromatography is performed with loading a total protein amount of about 51 μg to 153 μg of a Maintenance Dose of a pertuzumab trastuzmab FDC. In one embodiment the total protein loaded on the ion exchange chromatography is about 100 μg.

In one embodiment a method for evaluating a fixed dose composition comprising pertuzumab, and trastuzumab is provided, said method comprising:
a. Binding the antibodies to a ion exchange material using a loading buffer, wherein the pH of the loading buffer is between about pH 7.5 and about pH 7.65
b. Eluting the antibodies with an elution buffer, wherein the pH of the elution buffer is between about pH 7.5 and about pH 7.7
c. Selectively detecting charge variants of Pertuzumab and Trastuzumab in the composition.

In one embodiment the acidic variants, native forms and basic variants of trastuzumab and pertuzumab in a fixed dose combination are selectively detected.

In one embodiment the ion exchange chromatography is performed with a fixed dose combination of pertuzumab and trastuzumab that has been digested with carboxypeptidase B before loading on the chromatography column.

In one embodiment said fixed dose combination of pertuzumab and trastuzumab additionally comprises hyaluronidase. In one such embodiment the hyaluronidase is a recombinant human hyaluronidase. In one embodiment said hyaluronidase is rHuPH20. In one embodiment said pertuzumab and trastuzumab FDC comprises about 2000 U/mL rHuPH20. The pertuzumab trastuzumab FDC drug product is provided in two different dosages, i.e. a loading dose (LD) and a maintenance dose (MD). The LD and MD have the same total protein content and differ in the ratio of pertuzumab SC and trastuzumab SC drug substances. In one embodiment the method is useful to determine charge variants of a loading dose of a pertuzumab and trastuzumab FDC. In one embodiment the charge variants of both pertuzumab and trastuzumab are determined simultaneously, i.e. in the same method. In one embodiment the method is used to analyze charge variants of a pertuzumab trastuzumab FDC comprising 40 mg/mL trastuzumab and 80 mg/mL pertuzumab. In one embodiment said pertuzumab trastuzumab FDC additionally comprises rHuPH20 at 2000 U/mL. In one embodiment the method is useful to determine charge variants of a maintenance dose of a pertuzumab and trastuzumab FDC. In one embodiment the charge variants of both pertuzumab and trastuzumab are determined simultaneously, i.e. in the same method. In one embodiment the method is used to analyze charge variants of a pertuzumab trastuzumab FDC comprising 60 mg/mL and trastuzumab, 60 mg/mL pertuzumab. In one embodiment said pertuzumab trastuzumab FDC additionally comprises rHuPH20 at 2000 U/mL.

In one embodiment the native antibodies and their acidic and basic variants are eluted in a salt gradient from 1-100% (solvent B) over at least 44 minutes. In one embodiment the salt gradient is increased from 1 to 47% Solvent B over 43 minutes. In one embodiment the salt gradient is increased from about 1.8-103.4 mM NaCl. In another embodiment the salt gradient is increased from about 2 mM NaCl to about 94 mM NaCl.

In one embodiment, the mobile phase for the ion exchange chromatography comprises ACES buffer. In one embodiment mobile phase A and mobile phase B comprise ACES buffer. In one embodiment the ion exchange chromatography solvent A comprises about 10-50 mM, about 15-25 mM, about 18-22 mM or about 20 mM ACES. In one embodiment the ion exchange chromatography solvent B comprises about 10-50 mM, about 15-25 mM, about 18-22 mM or about 20 mM ACES and about 180-220 mM NaCl. In one embodiment solvent B comprises about 20 mM ACES.

(ii) Quantity/Protein Content Assay

UV Spectrophotometry is the typical method for determining total protein content of formulation samples. However, for a fixed dose combination (FDC) of two anti-HER2 antibodies, a different approach was required, as the conventional method does not allow separate and quantitative protein content analysis for each of the anti-HER2 antibodies in the FDC. Different chromatographic methods were tested, such as hydrophobic interaction (HIC) and reversed-phase chromatography (RPC). With regards to separate and quantitative protein content analysis of Pertuzumab Trastuzumab FDC, reversed phase chromatography proved to be the most suitable method.

Reversed-phase ultra-high-performance liquid chromatography (RP-UHPLC, RPC) separates molecules in solution according to their hydrophobicity. Separation is caused by the reversible, hydrophobic adsorption of molecules onto a non-polar stationary phase in the column. The adsorption of molecules to the solid support is driven by hydrophobic/non-polar interactions between the two moieties. The strength of interaction is determined by the number and location of functional groups on the molecule and stationary phase. In reversed-phase chromatography, non-polar molecules elute at higher retention times from the stationary phase than polar molecules. Since the two anti-HER2 antibodies Trastuzumab and pertuzumab have more than 93% sequence identity and differ only by 30 Da in total, a robust method was developed which provides reliable overall resolution and peak separation and has no significant sample carryover (i.e. carryover should not exceed 0.2% in the subsequent analysis). In addition, the content assays developed for the testing strategy took into account that the trastuzumab pertuzumab fixed dose combination is provided in two different dosages, i.e. loading dose and maintenance dose, which differ in the ratio of pertuzumab SC and trastuzumab SC drug substances. It was found by the present inventors that a phenyl-based column gave particularly robust results, and that the most critical parameters for a robust method were column temperature and flow rate. Phenyl-based RP-UHPLC columns are known in the art and can have the following groups: Ethyl phenyl with methyl side groups and an endcapped silica surface, Phenyl hexyl phase with extended (hexyl) ligand spacer methyl side groups, Ethyl phenyl ligand with steric protection (isobutyl) side groups, Hexyl biphenyl with methyl side groups, Biphenyl phase with methyl side groups, Oxygen activated phenyl ethyl phenyl phase with methyl side groups. HPLC columns with stationary phases modified with phenyl (e.g. single phenyl, biphenyl, diphenyl, phenyl hexyl, phenyl propyl) are readily available from most major column suppliers. One example of a phenyl column useful herein is an Agilent Zorbax RRHD 300-Diphenly column. In one embodiment said column is a 2.1×100 mm column.

Provided herein is a method for analyzing the protein content of a fixed dose combination (FDC) of two anti-HER2 antibodies comprising
a. Providing a RP-HPLC phenyl column
b. Loading the fixed dose combination (FDC) of two anti-HER2 antibodies on the RP-HPLC column
c. Separating the two anti-HER2 antibodies at a flow rate of 0.2-0.4 mL/min, wherein the column temperature is 64° C. to 76° C.

The RP-HPLC separation principle is based on hydrophobic association between the polypeptide solute and hydrophobic ligands on the chromatographic resin surface. The RP-HPLC column is usually part of a UHPLC system equipped with in-line vacuum degasser, autosampler with sample cooler, column heater and UV/VIS detector. Examples of suitable UHPLC systems are Waters Aquity and Thermo Ultimate 3000 RS.

The FDC of two anti-HER2 antibodies is loaded on the column by injecting a sample thereof into the RP-HPLC system. Usually the sample is diluted, for example to a concentration of approximately 0.5 to 5 mg/mL, or 1 mg/mL. It was found by the present inventors that a sample concentration of 1.0 mg/mL enables a good detectability of minor species without saturating the detector signal. In one embodiment the samples are diluted with formulation buffer. In one embodiment said formulation buffer comprises L-histidine, L-histidine hydrochloride monohydrate, L-methionine, α,α-trehalose dihydrate, sucrose, and polysorbate 20. By using formulation buffer as a diluent, the risk of altering the sample and reference solution upon using a different diluent than previously is non-existent. No relevant interference from formulation buffer with the RP-HPLC method was observed. In one embodiment the injection volume is 0.5 to 100 μL, 1-50 μL, 5-10 μL, or 10 μL. In one embodiment the injection volume is 10 μL In one embodiment the total protein load on the column is 10 μg.

Proteins bind to RP-HPLC columns in aqueous mobile phase and are eluted from the column by increasing the hydrophobicity of mobile phase. The proteins are then separated according to their hydrophobicity. In one embodiment of the method for analyzing the protein content of a fixed dose combination (FDC) of two anti-HER2 antibodies, the separation in step c) is achieved with a water-2-propanol/acetonitrile gradient. In one such embodiment the proteins are bound to the column in aqueous phase (eluent A) comprising water: 2-propanol (98:2)+0.1% Trifluoroacetic acid (TFA) and then eluted with increasing concentrations of an organic phase comprising acetonitrile. In one such embodiment the organic phase (eluent B) comprises 2-propanol: acetonitrile: eluent A (70:20:10). Due to the phenyl-based column type an improved specificity was achieved and new species were detected only with 2-propanol but not with pure acetonitrile. Different specificities were achieved because phenyl-based columns interact with the analyte via classic hydrophobic but also additional π-π-interactions. It has been shown in the literature that pure acetonitrile impedes these interactions, whereas 2-propanol does not (Yang, M., Fazio, S., Munch, D. & Drumm, P. Impact of methanol and acetonitrile on separations based on π-π interactions with a reversed-phase phenyl column. Journal of Chromatography A 1097, 124-129). However, considering the high viscosity of 2-propanol and the increased back-pressure associated with it, 20% acetonitrile was added to lower the back pressure in the system.

In one embodiment the aqueous mobile phase comprises 70% eluent A and 30% eluent B, wherein eluent A comprises water: 2-propanol (98:2)+0.1% Trifluoroacetic acid (TFA) and eluent B comprises 2-propanol: acetonitrile: eluent A (70:20:10). In one such embodiment the organic phase (eluent B) is increased to 55% eluent A and 45% eluent B. In one embodiment the gradient is increased to 45% eluent B over 15 minutes.

In one embodiment the organic phase (eluent B) is increased to 10% eluent A and 90% eluent B. In one embodiment the gradient is increased to 90% eluent B over 20 minutes.

Flow rates of 0.4 and 0.2 mL/min were tested and found to not have a significant impact on method performance. In one embodiment of the method for analyzing the protein content of a fixed dose combination (FDC) of two anti-HER2 antibodies, the flow rate in step c) is about 0.3 mL/min.

In one embodiment of the method for analyzing the protein content of a fixed dose combination (FDC) of two anti-HER2 antibodies, the antibodies are separated over 10 to 20 minutes. In one such embodiment, the antibodies are separated over 15 minutes. In one embodiment the antibodies are separated over 15 minutes at a flow rate of 0.3 mL/min.

In addition to the loading and elution (separation) step, RP-HPLC purification can include additional steps like equilibration, wash, and regeneration. In one embodiment the RP-HPLC phenyl column is equilibrated with 70% eluent A and 30% eluent B, wherein eluent A comprises water: 2-propanol (98:2)+0.1% Trifluoroacetic acid (TFA) and eluent B comprises 2-propanol: acetonitrile: eluent A (70:20:10). In one embodiment the RP-HPLC phenyl column is washed with 10% eluent A and 90% mobile phase B, wherein eluent A comprises water: 2-propanol (98:2)+0.1% Trifluoroacetic acid (TFA) and eluent B comprises 2-propanol: acetonitrile: eluent A (70:20:10).

In one embodiment of the method for analyzing the protein content of a fixed dose combination (FDC) of two anti-HER2 antibodies, the column temperature is 70° C.+−2° C. In comparison to room temperature, a column temperature of 70° C. leads to a higher reproducibility, removes tailing effects, shows a lower system back pressure and overall results in a better resolution and separation. Several column temperatures have been tested and 70° C. showed an improved peak pattern while not reaching the maximum temperature allowed for the system and column type. Temperatures of 64° C. and 76° C. and 66° C. and 74° C., respectively, were tested and found to not have a significant impact on method performance.

In one embodiment of the method for analyzing the protein content of a fixed dose combination (FDC) of two anti-HER2 antibodies, the phenyl column is a column selected from the group of Agilent Zorbax RRHD 300-Diphenyl column, Acclaim Phenyl-1 (Dionex), Pursuit® XRs Diphenyl, Pinnacle® Biphenyl, Zorbax® Eclipse® Plus Hexyl Phenyl, Ascentis Phenyl, BioResolve RP mAb Polyphenyl and Agilent AdvanceBio RP mAb Diphenyl. In one embodiment the phenyl column is a Agilent Zorbax RRHD 300-Diphenyl column. In another embodiment the phenyl column is a BioResolve RP mAb Polyphenyl column.

In one embodiment the proteins are detected by UV. In one embodiment the detection wavelength is 280 nm.

In one embodiment the fixed dose combination comprises Pertuzumab and Trastuzumab. In one embodiment the fixed dose combination of Pertuzumab and Trastuzumab additionally comprises hyaluronidase. In one such embodiment the hyaluronidase is a recombinant human hyaluronidase. In one embodiment said hyaluronidase is rHuPH20. In one embodiment said pertuzumab and trastuzumab FDC comprises about 2000 U/mL rHuPH20. The pertuzumab trastuzumab FDC drug product is provided in two different dosages, i.e. a loading dose (LD) and a maintenance dose (MD). The LD and MD have the same total protein content and differ in the ratio of pertuzumab SC and trastuzumab SC drug substances. In one embodiment the method is useful to determine the protein content of a loading dose of a pertuzumab and trastuzumab FDC. In one embodiment the protein content of both pertuzumab and trastuzumab are determined simultaneously, i.e. in the same method.

In one embodiment the method is used to analyze protein content of a pertuzumab trastuzumab FDC comprising 40 mg/mL trastuzumab and 80 mg/mL pertuzumab. In one embodiment said pertuzumab trastuzumab FDC additionally comprises rHuPH20 at 2000 U/mL. In one embodiment the method is useful to determine protein content of a maintenance dose of a pertuzumab and trastuzumab FDC. In one embodiment the protein content of both pertuzumab and trastuzumab are determined simultaneously, i.e. in the same method. In one embodiment the method is used to analyze protein content of a pertuzumab trastuzumab FDC comprising 60 mg/mL and trastuzumab, 60 mg/mL pertuzumab. In one embodiment said pertuzumab trastuzumab FDC additionally comprises rHuPH20 at 2000 U/mL.

### III. Anti-HER2 Antibodies and Compositions

(i) Anti-HER2 Antibodies

The HER2 antigen to be used for production of antibodies may be, e.g., a soluble form of the extracellular domain of a HER2 receptor or a portion thereof, containing the desired epitope. Alternatively, cells expressing HER2 at their cell surface (e.g. NIH-3T3 cells transformed to overexpress HER2; or a carcinoma cell line such as SK-BR-3 cells, see Stancovski et al. PNAS (USA) 88:8691-8695 (1991)) can be used to generate antibodies. Other forms of HER2 receptor useful for generating antibodies will be apparent to those skilled in the art.

Various methods for making monoclonal antibodies herein are available in the art. For example, the monoclonal antibodies may be made using the hybridoma method first described by Kohler et al., Nature, 256:495 (1975), by recombinant DNA methods (U.S. Pat. No. 4,816,567).

The anti-HER2 antibodies used in accordance with the present invention, pertuzumab and trastuzumab, are commercially available.

U.S. Pat. No. 6,949,245 describes production of exemplary humanized HER2 antibodies which bind HER2 and block ligand activation of a HER receptor.

Humanized HER2 antibodies specifically include trastuzumab as described in Table 3 of U.S. Pat. No. 5,821,337 expressly incorporated herein by reference and as defined herein; and humanized 2C4 antibodies such as pertuzumab as described and defined herein.

The humanized antibodies herein may, for example, comprise nonhuman hypervariable region residues incorporated into a human variable heavy domain and may further comprise a framework region (FR) substitution at a position selected from the group consisting of 69H, 71H and 73H utilizing the variable domain numbering system set forth in Kabat et al., Sequences of Proteins of Immunological Interest, 5th Ed. Public Health Service, National Institutes of Health, Bethesda, MD (1991). In one embodiment, the humanized antibody comprises FR substitutions at two or all of positions 69H, 71H and 73H.

An exemplary humanized antibody of interest herein comprises variable heavy domain complementarity determining residues GFTFTDYTMX (SEQ ID NO: 17), where X is preferably D or S; DVNPNSGGSIYNQRFKG (SEQ ID NO:18); and/or NLGPSFYFDY (SEQ ID NO:19), optionally comprising amino acid modifications of those CDR residues, e.g. where the modifications essentially maintain or improve affinity of the antibody. For example, an antibody variant for use in the methods of the present invention may have from about one to about seven or about five amino acid substitutions in the above variable heavy CDR sequences. Such antibody variants may be prepared by affinity maturation, e.g., as described below.

The humanized antibody may comprise variable light domain complementarity determining residues KASQDVSIGVA (SEQ ID NO:20); SASYX¹X²X³, where X¹ is preferably R or L, X² is preferably Y or E, and X³ is preferably T or S (SEQ ID NO:21); and/or QQYYIYPYT (SEQ ID NO:22), e.g. in addition to those variable heavy domain CDR residues in the preceding paragraph. Such humanized antibodies optionally comprise amino acid modifications of the above CDR residues, e.g. where the modifications essentially maintain or improve affinity of the antibody. For example, the antibody variant of interest may have from about one to about seven or about five amino acid substitutions in the above variable light CDR sequences. Such antibody variants may be prepared by affinity maturation, e.g., as described below.

The present application also contemplates affinity matured antibodies which bind HER2. The parent antibody may be a human antibody or a humanized antibody, e.g., one comprising the variable light and/or variable heavy sequences of SEQ ID Nos. 7 and 8, respectively (i.e. comprising the VL and/or VH of pertuzumab). An affinity matured variant of pertuzumab preferably binds to HER2 receptor with an affinity superior to that of murine 2C4 or pertuzumab (e.g. from about two or about four fold, to about 100 fold or about 1000 fold improved affinity, e.g. as assessed by ELISA. Exemplary variable heavy CDR residues for substitution include H28, H30, H34, H35, H64, H96, H99, or combinations of two or more (e.g. two, three, four, five, six, or seven of these residues). Examples of variable light CDR residues for alteration include L28, L50, L53, L56, L91, L92, L93, L94, L96, L97 or combinations of two or more (e.g. two to three, four, five or up to about ten of these residues).

Humanization of murine 4D5 antibody to generate humanized variants thereof, including trastuzumab, is described in U.S. Pat. Nos. 5,821,337, 6,054,297, 6,407,213, 6,639,055, 6,719,971, and 6,800,738, as well as Carter et al. PNAS (USA), 89:4285-4289 (1992). HuMAb4D5-8 (trastuzumab) bound HER2 antigen 3-fold more tightly than the mouse 4D5 antibody, and had secondary immune function (ADCC) which allowed for directed cytotoxic activity of the humanized antibody in the presence of human effector cells. HuMAb4D5-8 comprised variable light (V_{L}) CDR residues incorporated in a V_{L} κ subgroup I consensus framework, and variable heavy (V_{H}) CDR residues incorporated into a V_{H} subgroup III consensus framework. The antibody further comprised framework region (FR) substitutions as positions: 71, 73, 78, and 93 of the V_{H} (Kabat numbering of FR residues; and a FR substitution at position 66 of the V_{L} (Kabat numbering of FR residues). trastuzumab comprises non-A allotype human-γ 1 Fc region.

Various forms of the humanized antibody or affinity matured antibody are contemplated. For example, the humanized antibody or affinity matured antibody may be an antibody fragment. Alternatively, the humanized antibody or affinity matured antibody may be an intact antibody, such as an intact IgG1 antibody.

(ii) Pertuzumab Compositions

In one embodiment of a HER2 antibody composition, the composition comprises a mixture of a native pertuzumab antibody and one or more variants thereof. The preferred embodiment herein of a pertuzumab native antibody is one comprising the variable light and variable heavy amino acid sequences in SEQ ID Nos. 7 and 8, and most preferably comprising a light chain amino acid sequence of SEQ ID No. 11, and a heavy chain amino acid sequence of SEQ ID No. 12. In one embodiment, the composition comprises a mixture of the native pertuzumab antibody and an amino acid sequence variant thereof comprising an amino-terminal leader extension. Preferably, the amino-terminal leader extension is on a light chain of the antibody variant (e.g. on one or two light chains of the antibody variant). The main species HER2 antibody or the antibody variant may be an full length antibody or antibody fragment (e.g. Fab of F(ab=)2 fragments), but preferably both are full length antibodies. The antibody variant herein may comprise an amino-terminal leader extension on any one or more of the heavy or light chains thereof. Preferably, the amino-terminal leader extension is on one or two light chains of the antibody. The amino-terminal leader extension preferably comprises or consists of VHS—. Presence of the amino-terminal leader extension in the composition can be detected by various analytical techniques including, but not limited to, N-terminal sequence analysis, assay for charge heterogeneity (for instance, cation exchange chromatography or capillary zone electrophoresis), mass spectrometry, etc. The amount of the antibody variant in the composition generally ranges from an amount that constitutes the detection limit of any assay (preferably N-terminal sequence analysis) used to detect the variant to an amount less than the amount of the main species antibody. Generally, about 20% or less (e.g. from about 1% to about 15%, for instance from 5% to about 15%) of the antibody molecules in the composition comprise an amino-terminal leader extension. Such percentage amounts are preferably determined using quantitative N-terminal sequence analysis or cation exchange analysis (preferably using a high-resolution, weak cation-exchange column, such as a PROPAC WCX-10™ cation exchange column). Aside from the amino-terminal leader extension variant, further amino acid sequence alterations of the main species antibody and/or variant are contemplated, including but not limited to an antibody comprising a C-terminal lysine residue on one or both heavy chains thereof, a deamidated antibody variant, etc.

Moreover, the main species antibody or variant may further comprise glycosylation variations, non-limiting examples of which include antibody comprising a G1 or G2 oligosaccharide structure attached to the Fc region thereof, antibody comprising a carbohydrate moiety attached to a light chain thereof (e.g. one or two carbohydrate moieties, such as glucose or galactose, attached to one or two light chains of the antibody, for instance attached to one or more lysine residues), antibody comprising one or two non-glycosylated heavy chains, or antibody comprising a sialidated oligosaccharide attached to one or two heavy chains thereof etc.

The composition may be recovered from a genetically engineered cell line, e.g. a Chinese Hamster Ovary (CHO) cell line expressing the HER2 antibody, or may be prepared by peptide synthesis.

For more information regarding exemplary pertuzumab compositions, see U.S. Pat. Nos. 7,560,111 and 7,879,325 as well as US 2009/0202546A1.

(iii) Trastuzumab Compositions

The trastuzumab composition generally comprises a mixture of a main species antibody (comprising light and heavy chain sequences of SEQ ID NOS: 13 and 14, respectively), and variant forms thereof, in particular acidic variants (including deamidated variants). Preferably, the amount of such acidic variants in the composition is less than about 25%, or less than about 20%, or less than about 15%. See, U.S. Pat. No. 6,339,142. See, also, Harris et al., J. Chromatography, B 752:233-245 (2001) concerning forms of trastuzumab resolvable by cation-exchange chromatography, including Peak A (Asn30 deamidated to Asp in both light chains); Peak B (Asn55 deamidated to isoAsp in one heavy chain); Peak 1 (Asn30 deamidated to Asp in one light chain); Peak 2 (Asn30 deamidated to Asp in one light chain, and Asp102 isomerized to isoAsp in one heavy chain); Peak 3 (main peak form, or main species antibody); Peak 4 (Asp102 isomerized to isoAsp in one heavy chain); and Peak C (Asp102 succinimide (Asu) in one heavy chain).

(iv) Trastuzumab Pertuzumab Compositions in a Fixed Dose Combination

The present examples disclose extensive research on the various charge variants found in a trastuzumab pertuzumab fixed dose combination. The acceptance criteria were established based on clinical experience and the assumed impact on bioactivity/PK and safety/immunogenicity profile. The compositions provided herein are considered having the bioactivity and PK required for a safe biomedicine, with no added risk to immunogenicity and safety.

In one embodiment a composition comprising Pertuzumab and Trastuzumab is provided, wherein the composition comprises less than 23% of acidic pertuzumab variants selected from deamidation of HC-Asn-391, Fc sialic acid, and lysine glycation and trastuzumab variants deamidated at LC-Asn-30 and trastuzumab variants deamidated at HC-Asn-55, at least 28% of Pertuzumab native antibody, at least 16% of Trastuzumab native antibody and less than 12% trastuzumab with single isomerization of HC-Asp-102 to iso-aspartic acid at one heavy chain.

In one embodiment a composition comprising Pertuzumab and Trastuzumab is provided, wherein the composition comprises less than 23% of acidic pertuzumab variants selected from deamidation of HC-Asn-391, Fc sialic acid, and lysine glycation and trastuzumab variants deamidated at LC-Asn-30 and trastuzumab variants deamidated at HC-Asn-55, at least 38% of Pertuzumab native antibody, at least 16% of Trastuzumab native antibody and less than 9% trastuzumab with single isomerization of HC-Asp-102 to iso-aspartic acid at one heavy chain.

In one embodiment a composition comprising Pertuzumab and Trastuzumab is provided, wherein the composition comprises less than 21% of acidic pertuzumab variants selected from deamidation of HC-Asn-391, Fc sialic acid, and lysine glycation and trastuzumab variants deamidated at LC-Asn-30 and trastuzumab variants deamidated at HC-Asn-55, at least 28% of Pertuzumab native antibody, at least 23% of Trastuzumab native antibody and less than 12% trastuzumab with single isomerization of HC-Asp-102 to iso-aspartic acid at one heavy chain.

In one embodiment the composition comprising Pertuzumab, Trastuzumab and their charge variants is analyzed by an ion exchange chromatography. In one embodiment, the composition comprising Pertuzumab and Trastuzumab and their charge variants is analyzed with an ion exchange chromatography according to any of the above embodiments. In one embodiment the percentages of the native antibodies and the charge variants are equal to peak areas determined by ion exchange chromatography according to any of the above embodiments, wherein (i) the pertuzumab variant deamidated at HC-Asn-391, pertuzumab FC sialic acid variant, pertuzumab lysine glycation variant trastuzumab deamidated at LC-Asn-30 and trastuzumab deamidated at HC-Asn-55 elute in peaks 1 to 3 and thus the percentage of these variants within the composition is equal to the sum of peak areas 1 to 3, (ii) the pertuzumab native antibody elutes in peak 4 and thus the percentage of pertuzumab native antibody in the composition equals to the peak area of peak 4, (iii) the trastuzumab native antibody elutes in peak 7 and thus the percentage of trastuzumab native antibody in the composition equals to the peak area of peak 7, (iv) trastuzumab with single isomerization of HC-Asp-102 to iso-aspartic acid at one heavy chain elutes in peak 8 and thus the percentage of this variant in the composition equals to the peak area of peak 8.

In one embodiment a composition comprising Pertuzumab and Trastuzumab is provided, wherein the composition comprises less than 23% peak area for the sum of peaks 1 to 3, at least 28% peak area for peak 4 (Pertuzumab native antibody), at least 16% peak area for peak 7 (Trastuzumab native antibody) and less than 12% peak area for peak 8 as determined by a method described in any of the above embodiments. In one aspect, said method comprises the steps of:
a. Binding the antibodies to a ion exchange material using a loading buffer, wherein the pH of the loading buffer is between about pH 7.5 and about pH 7.65.
b. Eluting the antibodies with an elution buffer, wherein the pH of the elution buffer is between about pH 7.5 and about pH 7.7.

In one embodiment, the ion exchange material is a cation exchange material. In one embodiment, the cation exchange chromatography material is a strong cation exchange material. In one embodiment, the cation exchange material comprises sulfonate groups.

In one embodiment step b is performed with a salt gradient. In one embodiment the elution buffer comprises sodium. In one embodiment, the elution buffer comprises sodium chloride.

In one embodiment the method for evaluating a fixed dose composition comprising pertuzumab and trastuzumab above additionally comprises step:
c. Selectively detecting charge variants of pertuzumab and trastuzumab in the composition.

In one embodiment the method is performed at a temperature of 32-40° C. In one embodiment the composition comprising Pertuzumab and Trastuzumab additionally comprises rHuPH20.

In one embodiment the composition comprising Pertuzumab and Trastuzumab comprises 40 to 60 mg/mL Trastuzumab and 60-80 mg/mL Pertuzumab.

In one embodiment a composition comprising Pertuzumab and Trastuzumab is provided, wherein the composition comprises less than 23% peak area for the sum of peaks 1 to 3, at least 38% peak area for peak 4 (Pertuzumab native antibody), at least 16% peak area for peak 7 (Trastuzumab native antibody) and less than 9% peak area for peak 8 as determined by a method described in any of the above embodiments. In one aspect, said method comprises the steps of:
a. Binding the antibodies to a ion exchange material using a loading buffer, wherein the pH of the loading buffer is between about pH 7.5 and about pH 7.65.
b. Eluting the antibodies with an elution buffer, wherein the pH of the elution buffer is between about pH 7.5 and about pH 7.7.

In one embodiment, the ion exchange material is a cation exchange material. In one embodiment, the cation exchange chromatography material is a strong cation exchange material. In one embodiment, the cation exchange material comprises sulfonate groups.

In one embodiment step b is performed with a salt gradient. In one embodiment the elution buffer comprises sodium. In one embodiment, the elution buffer comprises sodium chloride.

In one embodiment the method for evaluating a fixed dose composition comprising pertuzumab and trastuzumab above additionally comprises step:
c. Selectively detecting charge variants of pertuzumab and trastuzumab in the composition.

In one embodiment the method is performed at a temperature of 32-40° C. In one embodiment the composition comprising Pertuzumab and Trastuzumab additionally comprises rHuPH20.

In one embodiment the composition comprising Pertuzumab and Trastuzumab comprises 40 to 60 mg/mL Trastuzumab and 60-80 mg/mL Pertuzumab.

In one embodiment a composition comprising Pertuzumab and Trastuzumab is provided, wherein the composition comprises less than 21% peak area for the sum of peaks 1 to 3, at least 28% peak area for peak 4 (Pertuzumab native antibody), at least 23% peak area for peak 7 (Trastuzumab native antibody) and less than 12% peak area for peak 8 as determined by a method described in any of the above embodiments. In one aspect, said method comprises the steps of:
a. Binding the antibodies to a ion exchange material using a loading buffer, wherein the pH of the loading buffer is between about pH 7.5 and about pH 7.65.
b. Eluting the antibodies with an elution buffer, wherein the pH of the elution buffer is between about pH 7.5 and about pH 7.7.

In one embodiment, the ion exchange material is a cation exchange material. In one embodiment, the cation exchange chromatography material is a strong cation exchange material. In one embodiment, the cation exchange material comprises sulfonate groups.

In one embodiment step b is performed with a salt gradient. In one embodiment the elution buffer comprises sodium. In one embodiment, the elution buffer comprises sodium chloride.

In one embodiment the method for evaluating a fixed dose composition comprising pertuzumab and trastuzumab above additionally comprises step:
c. Selectively detecting charge variants of pertuzumab and trastuzumab in the composition.

In one embodiment the method is performed at a temperature of 32-40° C. In one embodiment the composition comprising Pertuzumab and Trastuzumab additionally comprises rHuPH20.

In one embodiment the composition comprising Pertuzumab and Trastuzumab comprises 40 to 60 mg/mL Trastuzumab and 60-80 mg/mL Pertuzumab.

In one embodiment a composition comprising Pertuzumab and Trastuzumab is provided, wherein the composition comprises less than 22% of acidic pertuzumab variants selected from deamidation of HC-Asn-391, Fc sialic acid, and lysine glycation and trastuzumab variants deamidated at LC-Asn-30 and trastuzumab variants deamidated at HC-Asn-55, at least 29.2% of Pertuzumab native antibody, at least 21.8% of Trastuzumab native antibody and less than 5% trastuzumab with single isomerization of HC-Asp-102 to iso-aspartic acid at one heavy chain.

In one embodiment a composition comprising Pertuzumab and Trastuzumab is provided, wherein the composition comprises less than 22% of acidic pertuzumab variants selected from deamidation of HC-Asn-391, Fc sialic acid, and lysine glycation and trastuzumab variants deamidated at LC-Asn-30 and trastuzumab variants deamidated at HC-Asn-55, at least 39.4% of Pertuzumab native antibody, at least 21.8% of Trastuzumab native antibody and less than 4.1% trastuzumab with single isomerization of HC-Asp-102 to iso-aspartic acid at one heavy chain.

In one embodiment a composition comprising Pertuzumab and Trastuzumab is provided, wherein the composition comprises less than 19.8% of acidic pertuzumab variants selected from deamidation of HC-Asn-391, Fc sialic acid, and lysine glycation and trastuzumab variants deamidated at LC-Asn-30 and trastuzumab variants deamidated at HC-Asn-55, at least 29.2% of Pertuzumab native antibody, at least 31% of Trastuzumab native antibody and less than 5% trastuzumab with single isomerization of HC-Asp-102 to iso-aspartic acid at one heavy chain.

In one embodiment the composition comprising Pertuzumab and Trastuzumab comprises 40 to 60 mg/mL Trastuzumab and 60-80 mg/mL Pertuzumab.

In a further aspect of the invention the compositions provided herein are obtainable by a method comprising the following steps:
a. adding a pre-defined amount of pertuzumab to a compounding vessel
b. adding trastuzumab in a 1:1 Trastuzumab to Pertuzumab ratio or in a 1:2 Trastuzumab to Pertuzumab ratio
c. adding rHuPH20.

In one embodiment the 1:1 Trastuzumab to Pertuzumab ratio results in a composition comprising 60 mg/mL Trastuzumab and 60 mg/mL Pertuzumab. In one embodiment the 1:2 Trastuzumab to Pertuzumab ratio results in a composition comprising 40 mg/mL Trastuzumab and 80 mg/mL Pertuzumab. In one embodiment rHuPH20 is added to the composition to achieve a final concentration of 2000 U/ml rHuPH20.

### IV. Recombinant HER2 Extracellular Domains

It has been found by the present inventors that a modified HER2 ECD lacking subdomain IV can be produced with a three-dimensional conformation resembling the native HER2 ECD, when including a recombinant subdomain III which has been truncated at the C-terminus. In one such embodiment the modified HER2 ECD comprises SEQ ID NO: 1, SEQ ID NO: 2 and SEQ ID NO: 34. In one embodiment a modified HER2 ECD comprising SEQ ID NO. 24 is provided. In one embodiment a modified HER2 ECD having 99%, 98%, 97%, 96%, 95%, or 90% sequence identity to SEQ ID NO. 24 is provided.

In one embodiment the recombinant HER2 extracellular subdomains I, II, III are fused to a Fc domain. In one embodiment said Fc domain is a murine, rat, rabbit or porcupine Fc domain. In one embodiment a modified HER2 ECD comprising SEQ ID NO: 25, SEQ ID NO: 26 or SEQ ID NO: 27 is provided. In one embodiment a modified HER2 ECD having 99%, 98%, 97%, 96%, 95%, or 90% sequence identity to SEQ ID NO. 25 is provided. In one embodiment a modified HER2 ECD having 99%, 98%, 97%, 96%, 95%, or 90% sequence identity to SEQ ID NO. 26 is provided. In one embodiment a modified HER2 ECD having 99%, 98%, 97%, 96%, 95%, or 90% sequence identity to SEQ ID NO. 27 is provided.

In one embodiment a modified ECD comprising SEQ ID NO: 33, SEQ ID NO: 3 and SEQ ID NO: 4 is provided. In one embodiment the modified ECD comprises SEQ ID NO: 33, SEQ ID NO: 36, SEQ ID NO: 3 and SEQ ID NO: 4.

It has been found by the present inventors that a modified HER2 ECD lacking subdomain II can be produced with a three-dimensional conformation resembling the native HER2 ECD, when including a recombinant subdomain I which has been truncated at the C-terminus and replacing HER2 ECD subdomain II with EGFR subdomain II. In one embodiment a modified HER2 ECD is provided comprising SEQ ID NO. 29. In one embodiment a modified HER2 ECD having at least 99%, 98%, 97%, 96%, 95%, or 90% sequence identity to SEQ ID NO. 29 is provided. In one embodiment the recombinant HER2 extracellular subdomains I, III and IV and subdomain II of EGFR are fused to a Fc domain. In one embodiment said Fc domain is a murine, rat, rabbit or porcupine Fc domain. In any of the above embodiments the capture reagent for assessing binding of trastuzumab does not comprise a HER2 ECD subdomain II In one embodiment a recombinant HER2 extracellular domain comprising SEQ ID NO: 30, SEQ ID NO: 31 or SEQ ID NO: 32 is provided. In one embodiment a modified HER2 ECD having at least 99%, 98%, 97%, 96%, 95%, or 90% sequence identity to SEQ ID NO. 30 is provided. In one embodiment a modified HER2 ECD having at least 99%, 98%, 97%, 96%, 95%, or 90% sequence identity to SEQ ID NO. 31 is provided. In one embodiment a modified HER2 ECD having at least 99%, 98%, 97%, 96%, 95%, or 90% sequence identity to SEQ ID NO. 32 is provided.

The recombinant HER2 extracellular domains can be produced and purified by methods known in the art. In one embodiment a method of making a recombinant HER2 extracellular domain is provided, wherein the method comprises culturing a host cell comprising nucleic acid(s) encoding the recombinant HER2 extracellular domain, under conditions suitable for expression of the recombinant HER2 extracellular domain, and optionally recovering the recombinant HER2 extracellular domain from the host cell (or host cell culture medium). For recombinant production of the recombinant HER2 extracellular domain, nucleic acids encoding the recombinant HER2 extracellular domain, are isolated and inserted into one or more vectors for further cloning and/or expression in a host cell. Such nucleic acids may be readily isolated and sequenced using conventional procedures or produced by recombinant methods or obtained by chemical synthesis. Suitable host cells for cloning or expression of recombinant HER2 extracellular domain-encoding vectors include prokaryotic or eukaryotic cells described herein. For example, recombinant HER2 extracellular domain may be produced in bacteria. For expression of antibody fragments and polypeptides in bacteria, see, e.g., U.S. Pat. Nos. 5,648,237, 5,789,199, and 5,840,523. After expression, the recombinant HER2 extracellular domain may be isolated from the bacterial cell paste in a soluble fraction and can be further purified.

In addition to prokaryotes, eukaryotic microbes such as filamentous fungi or yeast are suitable cloning or expression hosts for recombinant HER2 extracellular domain-encoding vectors. Suitable host cells for the expression of recombinant HER2 extracellular domains are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. In one aspect, the host cell is eukaryotic, e.g., a Chinese Hamster Ovary (CHO) cell.

### V. Kits

The present invention also provides a kit for specifically quantifying the binding of an antibody binding to HER2 extracellular subdomain II in a fixed dose combination (FDC) of a first antibody binding to HER2 extracellular subdomain II and a second anti-HER2 antibody, the kit comprising:
(a) a container containing, as a capture reagent, a protein comprising SEQ ID NO: 1, SEQ ID NO: 2 and SEQ ID NO: 34.
(b) instructions for quantifying the binding of an antibody binding to HER2 extracellular subdomain II.

In one embodiment the capture reagent comprises SEQ ID NO. 24. In one embodiment the capture reagent has at least 99%, 98%, 97%, 96%, 95%, or 90% sequence identity to SEQ ID NO. 24.

In one embodiment the capture reagent comprises SEQ ID NO: 25, SEQ ID NO: 26 or SEQ ID NO: 27. In one embodiment the capture reagent has at least 99%, 98%, 97%, 96%, 95%, or 90% sequence identity to SEQ ID NO. 25. In one embodiment the capture reagent has at least 99%, 98%, 97%, 96%, 95%, or 90% sequence identity to SEQ ID NO. 26. In one embodiment the capture reagent has at least 99%, 98%, 97%, 96%, 95%, or 90% sequence identity to SEQ ID NO. 27.

In one embodiment said instructions additionally comprise instructions to correlate the binding of the first antibody binding to HER2 extracellular subdomain II to its potency.

In one embodiment the second antibody binds to a different epitope than the first antibody. In one embodiment the second antibody is an antibody binding to HER2 extracellular subdomain IV. In one embodiment the first antibody is pertuzumab. In one embodiment the second antibody is trastuzumab.

In one embodiment said fixed dose combination of pertuzumab and trastuzumab additionally comprises hyaluronidase. In one such embodiment the hyaluronidase is a recombinant human hyaluronidase. In one embodiment said hyaluronidase is rHuPH20. In one embodiment said pertuzumab and trastuzumab FDC comprises about 2000 U/mL rHuPH20.

The present invention also provides a kit for specifically quantifying the binding of an antibody binding to HER2 extracellular subdomain IV in a fixed dose combination (FDC) of an antibody binding to HER2 extracellular subdomain IV and a second anti-HER2 antibody, the kit comprising:
(a) a container containing, as a capture reagent, a protein comprising SEQ ID NO: 33, SEQ ID NO: 36, SEQ ID NO: 3 and SEQ ID NO: 4
(b) instructions for quantifying the binding of an antibody binding to HER2 extracellular subdomain IV.

In one embodiment the capture reagent comprises SEQ ID NO. 29. In one embodiment the capture reagent has at least 99%, 98%, 97%, 96%, 95%, or 90% sequence identity to SEQ ID NO. 29.

In one embodiment the capture reagent comprises SEQ ID NO: 30, SEQ ID NO: 31 or SEQ ID NO: 32. In one embodiment the capture reagent has at least 99%, 98%, 97%, 96%, 95%, or 90% sequence identity to SEQ ID NO. 30. In one embodiment the capture reagent has at least 99%, 98%, 97%, 96%, 95%, or 90% sequence identity to SEQ ID NO. 31. In one embodiment the capture reagent has at least 99%, 98%, 97%, 96%, 95%, or 90% sequence identity to SEQ ID NO. 32.

In one embodiment said instructions additionally comprise instructions to correlate the binding of an antibody binding to HER2 extracellular subdomain IV to its potency.

In one embodiment the second antibody binds to a different epitope than the first antibody. In one embodiment the second antibody is an antibody binding to HER2 extracellular subdomain II In one embodiment the first antibody is trastuzumab. In one embodiment the second antibody is pertuzumab.

In one embodiment said fixed dose combination of pertuzumab and trastuzumab additionally comprises hyaluronidase. In one such embodiment the hyaluronidase is a recombinant human hyaluronidase. In one embodiment said hyaluronidase is rHuPH20. In one embodiment said pertuzumab and trastuzumab FDC comprises about 2000 U/mL rHuPH20.

### VI. Manufacturing Methods

In one embodiment a method for making a composition is provided, comprising: (1) producing a fixed dose composition comprising pertuzumab, trastuzumab and one or more variants thereof, and (2) subjecting the composition so-produced to an analytical assay to evaluate the amount of the variant(s) therein, wherein the variant(s) comprise: (i) pertuzumab deamidated at HC-Asn-391, pertuzumab FC sialic acid variant, pertuzumab lysine glycation variant, trastuzumab deamidated at LC-Asn-30, trastuzumab deamidated at HC-Asn-55 (ii) pertuzumab native antibody, (iii) trastuzumab native antibody (vi) trastuzumab with single isomerization of HC-Asp-102 to iso-aspartic acid at one heavy chain.

In one embodiment, the variant(s) comprise (i) less than 23% of the following variants: pertuzumab deamidated at HC-Asn-391, pertuzumab FC sialic acid variant, and pertuzumab lysine glycation variant, trastuzumab deamidated at LC-Asn-30, trastuzumab deamidated at HC-Asn-55 (ii) at least 28% of pertuzumab native antibody, (iii) at least 16% of trastuzumab native antibody, (iv) less than 12% trastuzumab with single isomerization of HC-Asp-102 to iso-aspartic acid at one heavy chain.

In one embodiment, the variant(s) comprise (i) less than 23% of the following variants: pertuzumab deamidated at HC-Asn-391, pertuzumab FC sialic acid variant, pertuzumab lysine glycation variant, trastuzumab deamidated at LC-Asn-30, trastuzumab deamidated at HC-Asn-55 (ii) at least 38% of pertuzumab native antibody, (iii) at least 16% of trastuzumab native antibody, (iv) less than 9% trastuzumab with single isomerization of HC-Asp-102 to iso-aspartic acid at one heavy chain.

In one embodiment, the variant(s) comprise (i) less than 21% of the following variants pertuzumab deamidated at HC-Asn-391, pertuzumab FC sialic acid variant, and pertuzumab lysine glycation variant, trastuzumab deamidated at LC-Asn-30, trastuzumab deamidated at HC-Asn-55 (ii) at least 28% of pertuzumab native antibody, (iii) at least 23% of trastuzumab native antibody, (iv) less than 12% trastuzumab with single isomerization of HC-Asp-102 to iso-aspartic acid at one heavy chain.

In one embodiment said analytical assay is an ion exchange chromatography. In one embodiment said analytical assay is an ion exchange chromatography according to any of the above embodiments. In one embodiment the percentages are equal to peak areas determined by ion exchange chromatography according to any of the above embodiments, wherein (i) the pertuzumab variant deamidated at HC-Asn-391, pertuzumab FC sialic acid variant, pertuzumab lysine glycation variant trastuzumab deamidated at LC-Asn-30 and trastuzumab deamidated at HC-Asn-55 elute in peaks 1 to 3 and thus the percentage of these variants within the composition is equal to the sum of peak areas 1 to 3, (ii) the pertuzumab native antibody elutes in peak 4 and thus the percentage of pertuzumab native antibody in the composition equals to the peak area of peak 4, (iii) the trastuzumab native antibody elutes in peak 7 and thus the percentage of trastuzumab native antibody in the composition equals to the peak area of peak 7, (iv) trastuzumab with single isomerization of HC-Asp-102 to iso-aspartic acid at one heavy chain elutes in peak 8 and thus the percentage of this variant in the composition equals to the peak area of peak 8.

In one embodiment the amounts of the following additional variants are analyzed in the analytical assay: (v) pertuzumab with N-Terminal VHS on heavy and light chains, pertuzumab with C-terminal lysine at the heavy chain, trastuzumab with deamidation of HC-Asn-392, trastuzumab with lysine glycation and trastuzumab with increased Fc sialic acid content.

In one embodiment said analytical assay is an ion exchange chromatography. In one embodiment said analytical assay is an ion exchange chromatography according to any of the above embodiments. In one embodiment the percentages are equal to peak areas determined by ion exchange chromatography according to any of the above embodiments, wherein (vii) pertuzumab with N-Terminal VHS on heavy and light chains, pertuzumab with C-terminal lysine at the heavy chain, trastuzumab with deamidation of HC-Asn-392, trastuzumab with lysine glycation and trastuzumab with increased Fc sialic acid content elute in peaks 5-6. thus the percentage of these variants in the composition equals to the peak area of peaks 5-6.

In one embodiment the amounts of the following additional variants are analyzed in the analytical assay: (vi) trastuzumab with single isomerization of HC Asp102 to succinimide at one heavy chain and trastuzumab Fc oxidation.

In one embodiment said analytical assay is an ion exchange chromatography. In one embodiment said analytical assay is an ion exchange chromatography according to any of the above embodiments. In one embodiment the percentages are equal to peak areas determined by ion exchange chromatography according to any of the above embodiments, wherein (vi) trastuzumab with single isomerization of HC Asp102 to succinimide at one heavy chain and trastuzumab Fc oxidation. elute in peaks 9-10. Thus the percentage of these variants in the composition equals to the peak area of peaks 9-10.

In one embodiment the method is for making a composition that additionally comprises rHuPH20. In one embodiment the composition comprises 2000 U/ml rHuPH20. In one embodiment the method is for making a composition that comprises 40 to 60 mg/mL Trastuzumab and 60-80 mg/mL Pertuzumab. In one embodiment the composition comprises 40 mg/mL Trastuzumab and 80 mg/mL Pertuzumab. In one embodiment the composition comprises 60 mg/mL Trastuzumab and 60 mg/mL Pertuzumab.

In one embodiment step (1) of the method of making as described above comprises the following steps:
a. adding a pre-defined amount of pertuzumab to a compounding vessel
b. adding trastuzumab in a 1:1 Trastuzumab to Pertuzumab ratio or in a 1:2 Trastuzumab to Pertuzumab ratio
c. adding rHuPH20.

In one embodiment the 1:1 Trastuzumab to Pertuzumab ratio results in a composition comprising 60 mg/mL Trastuzumab and 60 mg/mL Pertuzumab. In one embodiment the 1:2 Trastuzumab to Pertuzumab ratio results in a composition comprising 40 mg/mL Trastuzumab and 80 mg/mL Pertuzumab.

In one embodiment rHuPH20 is added to the composition to achieve a final concentration of 2000 U/ml rHuPH20.

### VII. Selecting Patients for Therapy

Detection of HER2 expression or amplification can be used to select patients for treatment in accordance with the present invention. Several FDA-approved commercial assays are available to identify HER2-positive, HER2-expressing, HER2-overexpressing or HER2-amplified cancer patients. These methods include HERCEPTEST® (Dako) and PATHWAY® HER2 (immunohistochemistry (IHC) assays) and PathVysion® and HER2 FISH pharmDx™ (FISH assays). Users should refer to the package inserts of specific assay kits for information on the validation and performance of each assay.

For example, HER2 expression or overexpression may be analyzed by IHC, e.g. using the HERCEPTEST® (Dako). Paraffin embedded tissue sections from a tumor biopsy may be subjected to the IHC assay and accorded a HER2 protein staining intensity criteria as follows:

Score 0 no staining is observed or membrane staining is observed in less than 10% of tumor cells.

Score 1+a faint/barely perceptible membrane staining is detected in more than 10% of the tumor cells. The cells are only stained in part of their membrane.

Score 2+a weak to moderate complete membrane staining is observed in more than 10% of the tumor cells.

Score 3+a moderate to strong complete membrane staining is observed in more than 10% of the tumor cells.

Those tumors with 0 or 1+ scores for HER2 overexpression assessment may be characterized as HER2-negative, whereas those tumors with 2+ or 3+ scores may be characterized as HER2-positive.

Tumors overexpressing HER2 may be rated by immunohistochemical scores corresponding to the number of copies of HER2 molecules expressed per cell, and can been determined biochemically:
0=0-10,000 copies/cell,
1+=at least about 200,000 copies/cell,
2+=at least about 500,000 copies/cell,
3+=at least about 2,000,000 copies/cell.

Overexpression of HER2 at the 3+ level, which leads to ligand-independent activation of the tyrosine kinase (Hudziak et al., Proc. Natl. Acad. Sci. USA, 84:7159-7163 (1987)), occurs in approximately 30% of breast cancers, and in these patients, relapse-free survival and overall survival are diminished (Slamon et al., Science, 244:707-712 (1989); Slamon et al., Science, 235:177-182 (1987)).

The presence of HER2 protein overexpression and gene amplification are highly correlated, therefore, alternatively, or additionally, the use of in situ hybridization (ISH), e.g. fluorescent in situ hybridization (FISH), assays to detect gene amplification may also be employed for selection of patients appropriate for treatment in accordance with the present invention. FISH assays such as the INFORM™ (sold by Ventana, Arizona) or PathVysion® (Vysis, Illinois) may be carried out on formalin-fixed, paraffin-embedded tumor tissue to determine the extent (if any) of HER2 amplification in the tumor.

Most commonly, HER2-positive status is confirmed using archival paraffin-embedded tumor tissue, using any of the foregoing methods.

Preferably, HER2-positive patients having a 2+ or 3+ IHC score and/or who are FISH or ISH positive are selected for treatment in accordance with the present invention. Patients with 3+ IHC score and FISH/ISH positivity are particularly suitable for treatment in accordance with the present invention.

HER2 mutations associated with responsiveness to HER2-directed therapy have also been identified. Such mutations include, without limitation, insertions in exon 20 of HER2, deletions around amino acid residues 755-759 of HER2, any of the mutations G309A, G309E, S310F, D769H, D769Y, V777L, P780-Y781insGSP, V842I, R896C (Bose et al., Cancer Discov 2013; 3:1-14), as well as previously reported identical non-synonymous putative activating mutations (or indels) in COSMIC database found in two or more unique specimens.

See also U.S. Pat. No. 7,981,418 for alternative assays for screening patients for therapy with pertuzumab, and the Examples.

TABLE 1
SEQUENCES

| Description | SEQ ID NO | FIG. |
|---|---|---|
| HER2 domain I | 1 | 1 |
| HER2 domain II | 2 | 1 |
| HER2 domain III | 3 | 1 |
| HER2 domain IV | 4 | 1 |
| 2C4 variable light | 5 | 2A |
| 2C4 variable heavy | 6 | 2B |
| 574/pertuzumab variable light | 7 | 2A |
| 574/pertuzumab variable heavy | 8 | 2B |
| human V_{L} consensus framework | 9 | 2A |
| Human V_{H} consensus framework | 10 | 2B |
| pertuzumab light chain | 11 | 3A |
| pertuzumab heavy chain | 12 | 3B |
| trastuzumab light chain | 13 | 4A |
| trastuzumab heavy chain | 14 | 4B |
| Variant pertuzumab light chain | 15 | 5A |
| Variant pertuzumab heavy chain | 16 | 5B |
| GFTFTDYTMX | 17 | |
| DVNPNSGGSIYNQRFKG | 18 | |
| NLGPSFYFDY | 19 | |
| KASQDVSIGVA | 20 | |
| SASYX¹X²X³ | 21 | |
| QQYYIYPYT | 22 | |
| Recombinant HER2 extracellular domain II | 23 | |
| Capturing agent for anti-HER2 antibody binding to | 24 | |
| ECD domain II | | |
| Capturing agent for anti-HER2 antibody binding to | 25 | |
| ECD domain II connected to Fc domain | | |
| Capturing agent for anti-HER2 antibody binding to | 26 | |
| ECD domain II connected to Fc domain | | |
| Capturing agent for anti-HER2 antibody binding to | 27 | |
| ECD domain II connected to Fc domain | | |
| Recombinant HER2 extracellular domain IV | 28 | |
| Capturing agent for anti-HER2 antibody binding to | 29 | |
| ECD domain IV | | |
| Capturing agent for anti-HER2 antibody binding to | 30 | |
| ECD domain IV connected to Fc domain | | |
| Capturing agent for anti-HER2 antibody binding to | 31 | |
| ECD domain IV connected to Fc domain | | |
| Capturing agent for anti-HER2 antibody binding to | 32 | |
| ECD domain IV connected to Fc domain | | |
| Recombinant HER2 extracellular domain I | 33 | |
| Recombinant HER2 extracellular domain III | 34 | |
| Murine Fc domain | 35 | |
| EGFR ECD subdomain II | 36 | |

TABLE 2
LIST OF ABBREVIATIONS AND DEFINITIONs OF TERMS

| Abbreviation | Definition |
|---|---|
| ABTS | 2,2′-Azinobis [3-ethylbenzothiazoline-6-sulfonic |
| | acid]-diammonium salt |
| ACES | N-(2-Acetamido)-2-aminoethanesulfonic acid |
| BSA | bovine serum albumin |
| CDR | Complementarity determining region |
| CoA | certificate of analysis |
| CpB | Carboxypeptidase B |
| DPBS | Dulbecco's phosphate-buffered saline |
| EC₅₀ | half maximal effective concentration |
| ECD | extracellular domain |
| EGFR | epidermal growth factor receptor |
| ELISA | enzyme-linked immunosorbent assay |
| FDC | fixed-dose combination |
| FDC drug | pertuzumab-trastuzumab fixed-dose combination drug |
| product | product for subcutaneous injection |
| HER | human epidermal growth factor receptor |
| HER2 | human epidermal growth factor receptor 2 |
| HPLC | high performance liquid chromatography |
| HRP | horseradish peroxidase |
| HSR | Hill slope ratio |
| IE-HPLC | ion exchange high performance liquid chromatography |
| IgG | immunoglobulin G |
| iv | for intravenous injection |
| LAD | lower asymptote deviation |
| LC-MS | Liquid chromatography-mass spectrometry |
| LD | loading dose |
| mAB | Monoclonal antibody |
| MD | maintenance dose |
| NA | not applicable |
| OD | optical density |
| rHuPH20 | recombinant human hyaluronidase |
| RP-UHPLC | Reversed-phase ultra-high-performance liquid |
| | chromatography |
| R² | coefficient of determination |
| SC | subcutaneous |
| U | Unit |
| UAD | upper asymptote deviation |
| UV | ultraviolet |
| WFI | water for injection |

### Example 1: Pertuzumab-Trastuzumab FDC

Pertuzumab and trastuzumab, two of the active ingredients of FDC drug product LD and MD, are recombinant humanized monoclonal antibodies of the IgG1 subclass directed against the extracellular domains of HER2. rHuPH20, the third active ingredient of the FDC drug products, is a transiently active enzyme (recombinant human hyaluronidase) that acts as a local permeation enhancer, allowing for the subcutaneous delivery of therapeutics traditionally delivered intravenously.

The FDC drug product is provided as a sterile, colorless-to-slightly brownish solution for subcutaneous injection. It contains no preservatives. There are two formulations as described below:

Loading Dose: FDC Drug Product LD Each 20 mL single-dose vial contains 1200 mg (nominal) pertuzumab, 600 mg (nominal) trastuzumab and 2000 U/mL hyaluronidase (rHuPH20, vorhyaluronidase alfa) at target pH 5.5. The drug product is formulated as 80 mg/mL pertuzumab and 40 mg/mL trastuzumab. Excipients used in the formulation are L-histidine, L-histidine hydrochloride monohydrate, L-methionine, α,α-trehalose dihydrate, sucrose, and polysorbate 20.

Maintenance Dose: FDC Drug Product MD

Each 15 mL single-dose vial contains 600 mg (nominal) of pertuzumab, 600 mg (nominal) of trastuzumab and 2000 U/mL hyaluronidase (rHuPH20, vorhyaluronidase alfa) at target pH 5.5. The drug product is formulated as 60 mg/mL pertuzumab and 60 mg/mL trastuzumab. Excipients used in the formulation are L-histidine, L-histidine hydrochloride monohydrate, L-methionine, α,α-trehalose dihydrate, sucrose, and polysorbate 20.

### Example 2: Potency of Pertuzumab Trastuzumab FDC by Cell-Based Assays

This method determines the potency of pertuzumab and trastuzumab measuring their ability to inhibit proliferation of MDA-MB-175-VII or BT-474 cells, respectively. In a typical assay, 96-well microtiter plate(s) are seeded with MDA-MB-175-VII cells or BT-474 cells and incubated overnight at 37° C. with 5% carbon dioxide in a humidified incubator. After incubation, the medium is removed, and varying concentrations of Reference Standard, assay control, and sample(s) are added to the plate(s). The plate(s) are then incubated for 3 days, and the relative number of viable cells is quantitated indirectly using a redox dye, alamarBlue.

The fluorescence is measured using excitation at 530 nm and emission at 590 nm.

The alamarBlue dye is blue and nonfluorescent in its oxidized state, but it is reduced by the cell's intracellular environment to a pink form that is highly fluorescent. The changes in color and fluorescence are proportional to the number of viable cells. The results, expressed in RFU, are plotted against the antibody concentrations, and a parallel line analysis program is used to estimate the anti-proliferative activity of the FDC samples relative to the Reference Standard.

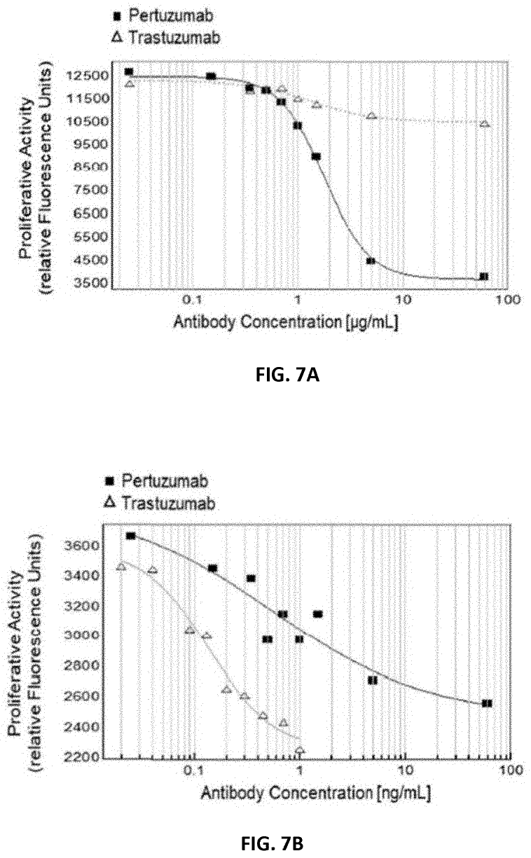

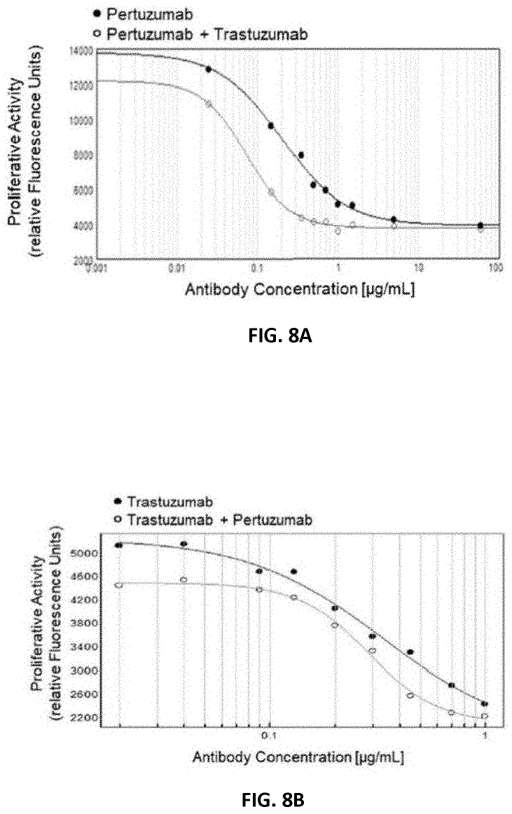

The cell-based assays are selectively sensitive for one or the other antibody in the FDC drug product, but not for both antibodies, as shown in FIGS. 7A and B. When analyzed individually, trastuzumab has an anti-proliferative activity on BT-474, but not on MDA-MD-175 VII cells, whereas pertuzumab has an anti-proliferative activity on MDA-MB-175 VII, but its activity on BT-474 cells is strongly shifted to higher concentrations. The difference in sensitivity for the two cell lines is likely based on the different HER2 expression levels (high and middle for BT-474 and MDA-MB-175 VII, respectively), rather than on differences in affinity for HER2. Also, HER3-expression levels and other potential parameters (e.g., presence or absence of HER3 endogenous ligand heregulin) involved in the overall anti-proliferative activity could contribute to the sensitivity difference. In addition, in the drug substance cell-based assays, the presence of one antibody influences the response of the other, masking potential quality changes occurring in one or the other antibody. Pertuzumab and trastuzumab have complementary mechanisms of action for disrupting HER2 signaling, resulting in higher anti-proliferative activity when both are present (FIGS. 8A and B). Although trastuzumab alone is not able to inhibit the proliferation of MDA-MB-175 VII cells in the pertuzumab anti-proliferation assay (FIGS. 7A and B), its addition to pertuzumab shifts the trastuzumab dose-response curve to lower EC50 values, reflecting higher potency when trastuzumab and pertuzumab are combined (FIG. 8A). Consequently, slight quality changes of pertuzumab in the FDC drug product will not be detected in the MDA-MB-175 VII anti-proliferation assay. Similar observations, although less pronounced, were made for pertuzumab in the BT-474 anti-proliferation assay (FIG. 8 B). Furthermore, slight quality changes of the antibodies in opposite directions might result in 100% potency.

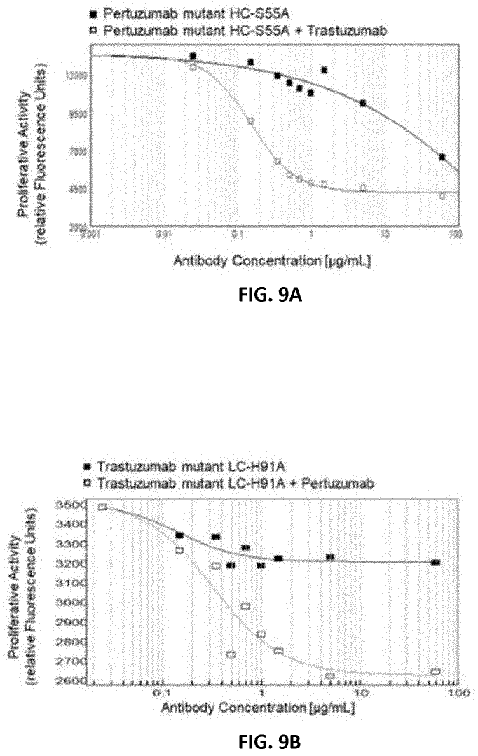

In order to demonstrate that substantial quality changes of either antibody in the FDC drug product cannot be detected in the anti-proliferation assays, pertuzumab and trastuzumab HER2 affinity-mutants with directed changes in the CDR (HC S55A and LC H91A mutation, respectively) were tested in the pertuzumab and trastuzumab anti-proliferations assays (FIGS. 9A and B). The mutants' greatly reduced affinity to HER2 correlates with their reduced anti-proliferative activities in their respective cell-based assays. The addition of pertuzumab to the trastuzumab mutant (or trastuzumab to the pertuzumab mutant) partially restores the dose-response curve shape and, therefore, the anti-proliferative activity.

In summary, based on the selective sensitivity, the complementary mechanisms, and the masking effects observed in the anti-proliferation assays, these assays are considered not to be suitable to detect relevant changes in the activity of either antibody in the co-formulation. These limitations disqualify the anti-proliferation assays for use in determining and controlling the bioactivity of the FDC drug product. Therefore, two selective potency ELISAs, which are not impacted by such cross-interferences, have been designed to control relevant changes in the binding activity of the two antibodies in the FDC drug product. The selectivity of the ELISAs is ensured by using the different binding epitopes of the HER2 receptor as primary binding targets.

### Example 3: Potency of Pertuzumab in FDC by ELISA

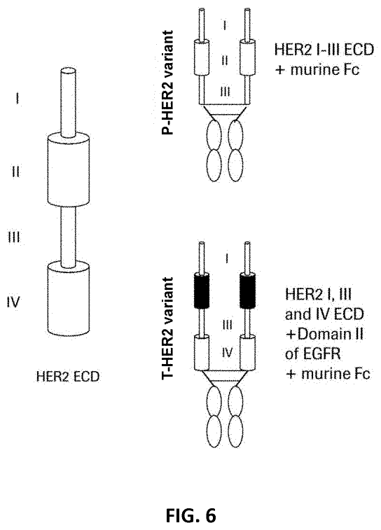

The potency of FDC drug product is controlled using two separate ELISAs. Here the ELISA controlling the bioactivity of the pertuzumab component of the FDC drug product is described. Pertuzumab is a monoclonal IgG1 antibody directed against HER2, specifically against the extracellular subdomain II of HER2. Upon binding, pertuzumab blocks activation of HER2 by preventing HER2 heterodimerization with ligand-activated members of the HER receptor family. This results in an inhibition of the downstream signaling pathway of HER2-overexpressing cells. The ELISA for pertuzumab determines the specific bioactivity as the ability of pertuzumab to specifically bind to its epitope of the recombinant HER2 (i.e., subdomain II). FIG. 6 depicts a schematic of the capture reagents used for the Pertuzumab ELISA and Trastuzumab ELISA (details see Example 6).

Binding is measured using a peroxidase-conjugated secondary antibody. A dose response curve generated for the sample and standard provides the basis for quantitation. For the ELISA, the actual protein content of pertuzumab (and not the total actual protein content of the FDC drug product) is considered in the dilution preparation. The ELISA for pertuzumab is used for both FDC drug product LD and MD.

Equipment and Material
96-well immuno plate (e.g., Maxisorp ELISA)
Absorbance plate reader
Computer with four-parameter data reduction software and parallelism analysis software (e.g., SoftMaxPro)
Microplate washer

Reagents
Pertuzumab coat reagent: recombinant HER2 extracellular domains I, II, III fused to a murine Fc; domain IV (containing the trastuzumab epitope) is depleted (SEQ ID NO: 27).
Detection antibody: HRP-conjugated goat anti-human antibody (specific for the F(ab′)2 portion of human IgG) (e.g. Jackson ImmunoResearch)
1×DPBS without calcium and magnesium
Purified water, e.g., Milli-Q.
BSA Fraction V
Tween 20
ABTS substrate solution
Phosphoric acid concentrated (85%)

Solutions

Note: Recipes are for nominal quantities of reagent and can be adjusted proportionally according to assay requirements.
WASH BUFFER: 1×DPBS, 0.05% Tween 20
ASSAY DILUENT: 1×DPBS, 0.05% Tween 20, 0.5% BSA Fraction V

COATING SOLUTION: Pertuzumab coat reagent (1 μg/mL) in 1×DPBS
DETECTION ANTIBODY: 0.8 mg/mL HRP-conjugated goat anti-human antibody
DETECTION SOLUTION: Prepare detection solution by diluting the detection antibody (0.8 mg/mL) in assay diluent to a concentration of 16 ng/mL. Prepare freshly before use.
STOP SOLUTION: 1 M phosphoric acid
REFERENCE STANDARD: FDC MD reference standard

Coating Plates
Transfer 100 μL of coating solution to each well of microtiter plates.
Incubate coated plates for 30-60 minutes at 2° C.-8° C.

Blocking Plates
Remove the excess of coating solution by washing all coated plates three times with 300 μL/well of wash buffer.
Block all plates by adding 100 μL of assay diluent to each well.
Incubate the plates for 60-90 minutes at ambient temperature under gentle shaking.
Wash blocked plates three times with 300 μL/well wash buffer.

Sample Transfer
Transfer 100 μL/well FDC reference standard, product control and sample dilutions to the wells of the immunoplate.
Incubate plates for 60-90 minutes at ambient temperature under gentle shaking.

Detection
Transfer 100 μL of detection solution (at 16 ng/mL) to each well of the plates.
Incubate plates for 30-90 minutes at ambient temperature under gentle shaking.
Wash plates three times with 300 μL/well of wash buffer.

Substrate Transfer and Measurement
Transfer 100 μL/well of ABTS substrate solution to each well of the plates.
Incubate plates at ambient temperature for 20-35 minutes under gentle shaking.
To stop the reaction, transfer 100 μL/well of stop solution to each well of the plates.
Mix plates by mild agitation for at least 1 minute.
Within 30 minutes, measure OD values at a wavelength of 405 nm (reference wavelength of 490 nm) on an absorbance plate reader.

Evaluation
Calculate the OD value of each well as follows: OD (405 nm)-OD (490 nm)

Where: OD (405 nm): detection absorbance at 405 nm, OD (490 nm): reference absorbance at 490 nm
Average the OD values of replicates to determine the mean OD.
Generate dose-response curves for standard, product control and sample(s) by plotting mean OD (y) against concentration of pertuzumab antibody concentration in ng/mL (x).
Apply non-linear regression using the following four-parameter equation:

$$y = D + \frac{A - D}{1 + {[ \frac{x}{C} ]}^{B}}$$

Where:

A: lower asymptote

B: Hill slope

C: EC₅₀ value

D: upper asymptote
Calculate the R₂ of standard, product control and sample curves.
Calculate the Standard delta OD as follows:

Standard delta OD=(Mean Maximum OD of standard)−(Mean Minimum OD of standard)
Determine the maximal OD as follows:

The maximal OD value is the maximal OD value at 405 nm obtained within all replicates of the dose-response curve.

Calculation of Potency
Calculate a common set of Hill slope, upper asymptote and lower asymptote for standard and sample (or product control) curves using four-parameter parallel line analysis.
The resulting curve equations for standard and sample (or product control) are:

$${y}_{standard} = D + \frac{A - D}{1 + {[ \frac{x}{{C}_{standard}} ]}^{B}} \qquad {y}_{sample} = D + \frac{A - D}{1 + {[ \frac{ρ x}{{C}_{sample}} ]}^{B}}$$

Where:

A=common lower asymptote

B=common Hill slope

C_{standard}=standard EC₅₀ value

D=common upper asymptote

ρ=potency of sample and product control relative to reference standard
Calculate relative potency as follows:

Relative Potency=ρ×Activity of reference Standard

Reference Standard Potency Assignment

The pertuzumab potency of FDC drug product is based on pertuzumab protein content instead of total protein content of the FDC drug product. Therefore, the potency measurement is independent from the ratio of the two molecules in the FDC drug product and one single molecule reference standard can be used to determine the potency of FDC drug product MD and LD samples. The FDC MD reference standard was selected as potency reference standard.

Details for the potency assignment of the FDC MD reference standard are provided in the following:
The potency was set to 1.00×10⁴ U/mg.
The pertuzumab potency determination by ELISA was performed relative to the commercial pertuzumab IV reference standard anti2C4907-2.
The trastuzumab potency determination by ELISA was performed relative to the commercial trastuzumab SC reference standard G005.03EP1

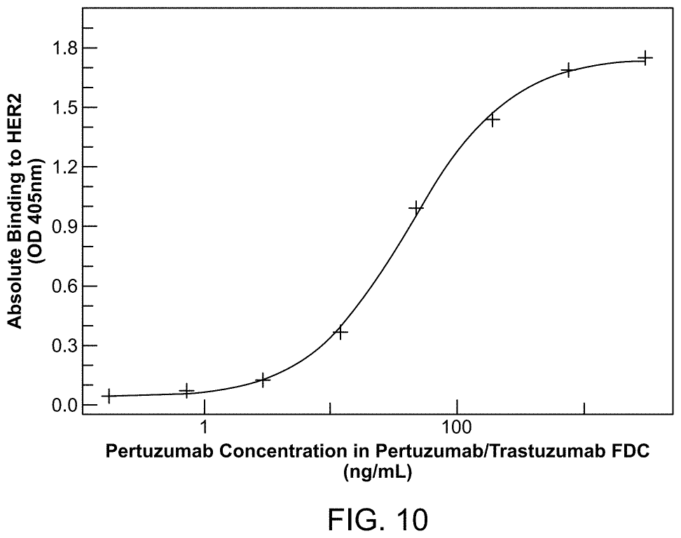

Results: The binding of pertuzumab in the pertuzumab trastuzumab Fixed Dose combination was analyzed in the pertuzumab ELISA assay. A representative dose-response curve is depicted in FIG. 10.

### Example 4: Specificity of Pertuzumab ELISA

To assess the specificity of the pertuzumab ELISA of Example 3, formulation buffers and structurally related molecules were tested at the highest assay concentration in duplicate on a single plate. In case interference with structurally related molecules was observed (mean value of the replicates is higher than three times the lower asymptote mean value of the reference standard dose-response curve), an escalation to full dose-response curve with one reportable result determination (n=1) has been performed.

The results demonstrate that the pertuzumab ELISA is specific for pertuzumab:
Both rHuPH20-containing FDC LD and MD formulation buffers showed no interference with the assay, demonstrating the suitability of the assay for the analysis of FDC drug product samples formulated in these matrices.
Structurally related molecules (except pertuzumab, see below), including trastuzumab, did not interfere with the pertuzumab ELISA. This is shown by the mean OD values of the replicates that are lower than three times the lower asymptote OD mean value of the reference standard dose-response curve.
As expected, pertuzumab SC drug substance formulated in FDC drug product formulation and pertuzumab IV showed interference in the pertuzumab ELISA since they bind to the same HER2 Domain II

The results are shown in Table 3.

TABLE 3
SPECIFICITY OF ELISA FOR PERTUZUMAB

| Sample | | Relative | OD Mean of |
|---|---|---|---|
| Name/Description | Response Detectable | Potency (%) | Replicates |
| Pertuzumab IV | Yes | 92 | NA |
| Pertuzumab SC | Yes | 93 | NA |
| Trastuzumab IV | No | NA | NA |
| Trastuzumab SC | No | NA | NA |
| Trastuzumab | No | NA | NA |
| Emtansine | | | |
| rHuPH20-Containing | No | NA | 0.07 |
| FDC LD Formulation | | | |
| Buffer | | | |
| rHuPH20-Containing | No | NA | 0.07 |
| FDC MD Formulation | | | |
| Buffer | | | |
| Epoetin Beta | No | NA | 0.07 |
| Peginterferon Alfa-2a | No | NA | 0.07 |
| Interferon Alfa-2a | No | NA | 0.07 |
| Methoxy Polyethylene | No | NA | 0.08 |
| Glycol-Epoetin Beta | | | |
| Atezolizumab | No | NA | 0.07 |
| Ocrelizumab | No | NA | 0.07 |
| Rituximab IV | No | NA | 0.09 |
| Bevacizumab | No | NA | 0.07 |
| Tocilizumab SC | No | NA | 0.07 |
| Etrolizumab | No | NA | 0.07 |
| Emicizumab | No | NA | 0.07 |
| Vorhyaluronidase Alfa | No | NA | 0.07 |
| Lebrikizumab | No | NA | 0.07 |
| Satralizumab | | NA | 0.07 |
| PEG-Filgrastim | No | NA | 0.07 |
| Filgrastim | No | NA | 0.08 |
| Polatuzumab | No | NA | 0.07 |
| Lower Asymptote | NA | NA | 0.08 |
| Mean Value of the | | | |
| Reference Standard | | | |
| Dose-Response Curve | | | |
| 3 Times the Lowest | NA | NA | 0.23 |
| Value of Reference | | | |
| Standard | | | |

### Example 5: Robustness of Pertuzumab ELISA

The robustness of the pertuzumab ELISA was assessed by deliberate variation of assay parameters that are a potential source of variation in practice. The robustness results were evaluated by comparing the obtained dose-response curve parameters, system suitability and similarity criteria with the method procedure condition. Overall robustness results are summarized in table 4.

TABLE 4
ROBUSTNESS RESULTS FOR PERTUZUMAB ELISA.

| Robustness Parameter | Test Condition | | Result |
|---|---|---|---|
| Coat Reagent | 0.5, 1, 1.5 | μg/mL | The assay tolerates coat reagent |
| Concentration | | | concentration ranging 1-1.5 μg/mL |
| Coating Incubation Time | 30, 60, 90 | minutes | The assay tolerates coating incubation time |
| | | | ranging 30-60 minutes |
| Blocking Incubation Time | 30, 60, 90 | minutes | The assay tolerates blocking incubation |
| | | | time ranging 60-90 minutes |
| FDC Drug Product | 30, 60, 90 | minutes | The assay tolerates FDC drug product |
| Incubation Time | | | incubation time ranging 60-90 minutes |
| Detection Antibody Batches | 3 | batches | The assay tolerates the use of different |
| | | | detection antibody batches originating from |
| | | | Jackson ImmunoResearch |
| Detection Antibody | 30, 60, 90 | minutes | The assay tolerates detection antibody |
| Incubation Time | | | incubation time ranging 30-90 minutes |
| Substrate Incubation Time | 20, 30, 40 | minutes | The assay tolerates substrate incubation |

| | | |
|---|---|---|
| | | time ranging 20-35 minutes |
| Use of Different Readers | SpectraMax M5, i3x | The assay tolerates the use of both |
| | | molecular devices SpectraMax M5 and i3x |

### Example 6: Potency of Trastuzumab in FDC by ELISA

The potency of FDC drug product is controlled using two similar ELISAs. This section describes the ELISA controlling the bioactivity of the trastuzumab component of the FDC drug product. Trastuzumab is a monoclonal IgG1 antibody directed against HER2, specifically against the extracellular subdomain IV of HER2. Upon binding, trastuzumab blocks activation of HER2 by preventing its homodimerization and shedding of HER2 extracellular domain.

This results in an inhibition of the downstream signaling pathway of HER2-overexpressing cells. The ELISA for trastuzumab determines the specific bioactivity as the ability of trastuzumab to specifically bind to its epitope of the recombinant HER2 (i.e., subdomain IV). FIG. 6 depicts a schematic of the capture reagent used for the Trastuzumab ELISA.

Binding is measured using a peroxidase-conjugated secondary antibody. A dose-response curve generated for the sample and standard provides the basis for quantitation. For the ELISA, the actual protein content of trastuzumab (and not the total actual protein content of the FDC drug product) is considered in the dilution preparation. The ELISA for trastuzumab is used for both FDC drug product LD and MD.

The reagents, buffers and procedures are as outlined in example 3, except for the coat reagent and coating solution:
Trastuzumab coat reagent: recombinant HER2 extracellular domains I, III, IV fused to a murine Fc; domain II is replaced by structurally related domain II of EGFR, which is not able to bind pertuzumab (SEQ ID NO: 32).
COATING SOLUTION: Trastuzumab coat reagent (1 μg/mL) in 1×DPBS

Evaluation
Calculate the OD value of each well as follows: OD (405 nm)-OD (490 nm)

Where: OD (405 nm): detection absorbance at 405 nm, OD (490 nm): reference absorbance at 490 nm
Average the OD values of replicates to determine the mean OD.
Generate dose-response curves for standard, product control and sample(s) by plotting mean OD (y) against concentration of trastuzumab antibody concentration in ng/mL (x).
Apply non-linear regression using the following four-parameter equation:

$$y = D + \frac{A - D}{1 + {[ \frac{x}{C} ]}^{B}}$$

Where:

A: lower asymptote

B: Hill slope

C: EC₅₀ value

D: upper asymptote
Calculate the R2 of standard, product control and sample curves.
Calculate the Standard delta OD as follows:

Standard delta OD=(Mean Maximum OD of standard)−(Mean Minimum OD of standard)
Determine the maximal OD as follows:

The maximal OD value is the maximal OD value at 405 nm obtained within all replicates of the dose-response curve.

Calculation of Potency
Calculate a common set of Hill slope, upper asymptote and lower asymptote for standard and sample (or product control) curves using four-parameter parallel line analysis.
The resulting curve equations for standard and sample (or product control) are:

$${y}_{standard} = D + \frac{A - D}{1 + {[ \frac{x}{{C}_{standard}} ]}^{B}} \qquad {y}_{sample} = D + \frac{A - D}{1 + {[ \frac{ρ x}{{C}_{sample}} ]}^{B}}$$

Where:

A=common lower asymptote

B=common Hill slope

C_{standard}=standard EC₅₀ value

D=common upper asymptote

ρ=potency of sample and product control relative to reference standard
Calculate relative potency as follows:

Relative Potency=ρ×Activity of reference Standard

Reference Standard Potency Assignment

The trastuzumab potency of FDC drug product are based on trastuzumab protein content instead of total protein content of the FDC drug product. Therefore, the potency measurement is independent from the ratio of the two molecules in the FDC drug product and one single molecule reference standard can be used to determine the potency of FDC drug product MD and LD samples. The FDC MD reference standard was selected as potency reference standard.

Details for the potency assignment of the FDC MD reference standard are provided in the following:
The potency was set to 1.00×10⁴ U/mg.
The pertuzumab potency determination by ELISA was performed relative to the commercial pertuzumab IV reference standard anti2C4907-2.
The trastuzumab potency determination by ELISA was performed relative to the commercial trastuzumab SC reference standard G005.03EP1.

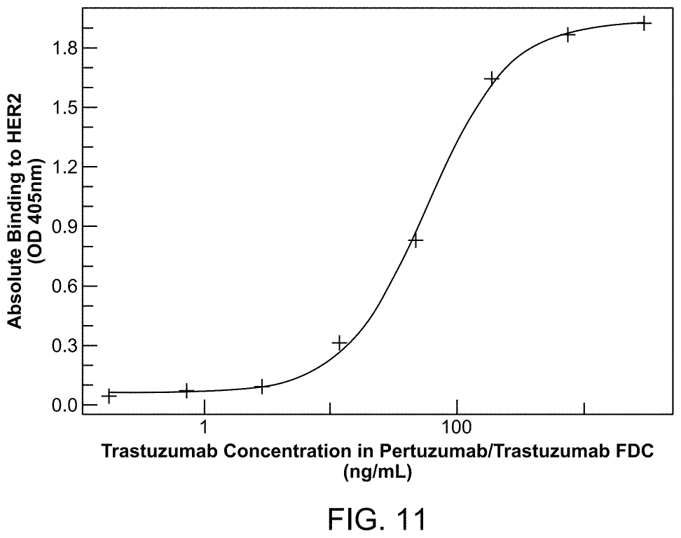

Results: The binding of trastuzumab in the pertuzumab trastuzumab Fixed Dose combination was analyzed in the trastuzumab ELISA assay. A representative dose-response curve is depicted in FIG. 11.

### Example 7: Specificity of Trastuzumab ELISA

To assess the specificity of the trastuzumab ELISA, formulation buffers and structurally related molecules were tested at the highest assay concentration in duplicate on a single plate. In case interference with structurally related molecules was observed (mean value of the replicates is higher than three times the lower asymptote mean value of the reference standard dose-response curve), an escalation to full dose-response curve with one reportable result determination (n=1) has been performed.

The results demonstrate that the trastuzumab ELISA is specific for trastuzumab:
Both rHuPH20-containing FDC LD and MD formulation buffers showed no interference with the assay, demonstrating the suitability of the assay for the analysis of FDC drug product samples formulated in these matrices.
Structurally related molecules (except trastuzumab, see below), including pertuzumab, did not interfere with the trastuzumab ELISA. This is shown by the mean OD values of the replicates that are lower than three times the lower asymptote OD mean value of the reference standard dose-response curve.
As expected, trastuzumab (IV and SC) and trastuzumab emtansine showed interference in the trastuzumab ELISA since they bind to the same HER2 Domain IV.

The results are shown in Table 5.

TABLE 5
SPECIFICITY OF ELISA FOR TRASTUZUMAB

| Sample | | Relative | OD Mean of |
|---|---|---|---|
| Name/Description | Response Detectable | Potency (%) | Replicates |
| Trastuzumab IV | Yes | 88 | NA |
| Trastuzumab SC | Yes | 92 | NA |
| Trastuzumab | Yes | 76 | NA |
| Emtansine | | | |
| Pertuzumab IV | No | NA | NA |
| Pertuzumab SC | No | NA | NA |
| rHuPH20-Containing | No | NA | 0.03 |
| FDC LD Formulation | | | |
| Buffer | | | |
| rHuPH20-Containing | No | NA | 0.03 |
| FDC MD Formulation | | | |
| Buffer | | | |
| Epoetin Beta | No | NA | 0.03 |
| Peginterferon Alfa-2a | No | NA | 0.03 |
| Interferon Alfa-2a | No | NA | 0.04 |
| Methoxy Polyethylene | No | NA | 0.04 |
| Glycol-Epoetin Beta | | | |
| Atezolizumab | No | NA | 0.03 |
| Ocrelizumab | No | NA | 0.03 |
| Rituximab IV | No | NA | 0.03 |
| Bevacizumab | No | NA | 0.03 |
| Tocilizumab SC | No | NA | 0.03 |
| Etrolizumab | No | NA | 0.03 |
| Emicizumab | No | NA | 0.03 |
| Vorhyaluronidase Alfa | No | NA | 0.03 |
| Lebrikizumab | No | NA | 0.03 |
| Satralizumab | No | NA | 0.03 |
| PEG-Filgrastim | No | NA | 0.03 |
| Filgrastim | No | NA | 0.04 |
| Polatuzumab | No | NA | 0.03 |
| Lower Asymptote | NA | NA | 0.04 |
| Mean Value of the | | | |
| Reference Standard | | | |
| Dose-Response Curve | | | |
| 3 Times the Lowest | NA | NA | 0.11 |
| Value of Reference | | | |
| Standard | | | |

### Example 8: Robustness of Trastuzumab ELISA

The robustness of the trastuzumab ELISA was assessed by deliberate variation of assay parameters that are a potential source of variation in practice. The robustness results were evaluated by comparing the obtained dose-response curve parameters, system suitability and similarity criteria with the method procedure condition. Overall robustness results are summarized in table 6.

TABLE 6
ROBUSTNESS RESULTS FOR TRASTUZUMAB ELISA.

| Robustness Parameter | Test Condition | | Result |
|---|---|---|---|
| Coat Reagent | 0.5, 1, 1.5 | μg/mL | The assay tolerates coat reagent |
| Concentration | | | concentration ranging 1-1.5 μg/mL |
| Coating Incubation Time | 30, 60, 90 | minutes | The assay tolerates coating incubation time |
| | | | ranging 30-60 minutes |
| Blocking Incubation Time | 30, 60, 90 | minutes | The assay tolerates blocking incubation |
| | | | time ranging 60-90 minutes |
| FDC Drug Product | 30, 60, 90 | minutes | The assay tolerates FDC drug product |
| Incubation Time | | | incubation time ranging 60-90 minutes |
| Detection Antibody Batches | 3 | batches | The assay tolerates the use of different |
| | | | detection antibody batches originating from |
| | | | Jackson ImmunoResearch |
| Detection Antibody | 30, 60, 90 | minutes | The assay tolerates detection antibody |
| Incubation Time | | | incubation time ranging 30-90 minutes |
| Substrate Incubation Time | 20, 30, 40 | minutes | The assay tolerates substrate incubation |

| | | |
|---|---|---|
| | | time ranging 20-35 minutes |
| Use of Different Readers | SpectraMax M5, i3x | The assay tolerates the use of both |
| | | molecular devices SpectraMax M5 and i3x |

### Example 9: Development of IEC to Analyze FDC Charge Variants

Various ion exchange chromatography protocols have been tested in order to resolve the FDC charge variants. The following parameters have been tested: column type, buffer type and concentration, salt concentration, flow rate, injection volume, pH value, column temperature and gradient profile.

The test method is developed to separate and determine the relative abundance (in % of total peak area) of the following peaks/peak groups:
Sum of Peaks 1-3
Peak 4 (Main Peak Pertuzumab)
Sum of Peaks 5-6
Peak 7 (Main Peak Trastuzumab)
Peak 8
Sum of Peaks 9-10

The FDC IE-HPLC method has been developed and optimized to enable the best achievable separation of pertuzumab and trastuzumab charge variants. From analyzing Trastuzumab SC and Pertuzumab SC separately, the expected charge variants can be extrapolated. Perjeta SC (lot GB0005, c=120 mg/mL) and Herceptin SC (lot P0003, c=120 mg/mL) were used individually as well as co-mixtures to perform the experiments.

In a first step, the registered IE-HPLC methods of the individual molecules Perjeta IV and Herceptin IV/SC were tested. These methods have been published e.g. in Zephania W. Kwong Glover et al, Compatibility and Stability of Pertuzumab and Trastuzumab Admixtures in i.v. Infusion Bags for Coadministration, Pharmaceutical Biotechnology, Vol. 02, Issue 3, P794-812, Mar. 1, 2013, DOI:https://doi.org/10.1002/jps.23403. In these methods, a weak cation exchange column (WCX-10) is used (see Table 7, Methods 1 and 2). In a next step, the ProPac WCX-10 column was tested with operating conditions which were successful for another mAb product bearing a similar pI value as pertuzumab/trastuzumab (see Method 3 in table 7). In a next step, a strong cation exchange column was used, and different buffers and pH values tested. The used parameters and results are summarized below in table 7.

In a next step, different columns were screened. The best resolution was achieved with a strong cation exchange column (Mab PAC SCX-10). Different buffers and pH values were tested (Methods 4 to 6). The used parameters and results are summarized in table 7. A further series was conducted based on Method 6, which gave the best results (see table 8).

Results

Method 1: When analyzing the pertuzumab trastuzumab FDC with the conditions of method 1, the resolution of the peaks was not satisfactory for the requirements of a product release assay: The resolution between Peak 7 (Main peak trastuzumab) and Peak 8 (IsoAsp102 of trastuzumab) was poor and the basic region of pertuzumab was overlapping with the trastuzumab main peak.

Method 2: When analyzing the pertuzumab trastuzumab FDC with the conditions of method 2, the resolution of the peaks was not satisfactory for the requirements of a product release assay: The basic region of pertuzumab was completely overlapping with trastuzumab main peak (Peak 7) and with Peak8 and is therefore not acceptable.

Method 3: Both main peaks of trastuzumab and pertuzumab could be separated and only minor overlaps of the basic region of pertuzumab was observed. However, the acidic region of trastuzumab was overlapping with the main peak of pertuzumab.

Method 4: Overlap of the basic region of pertuzumab with the main peak of trastuzumab and the IsoAsp102 peak of trastuzumab (Peak8)

Method 5: 1: pH 7.5: Good separation of both main peaks and Peak 8, only minor overlaps of the basic region of pertuzumab with the main peak of trastuzumab

Method 5: 2: pH 8.0: Good separation of Peak 8, but stronger overlap of the basic region of pertuzumab with the main peak of trastuzumab compared to method 5 with pH 7.5

Method 6: Good separation of all species of interest.

TABLE 7
DEVELOPMENT OF IEC PROTOCOL TO ANALYZE FDC CHARGE VARIANTS.

| | Method 1 | Method 2 | Method 3 | Method 4 | Method 5 | Method 6 |
|---|---|---|---|---|---|---|
| Column | ProPac WCX-10 | | | MabPac SCX-10 | | |
| Solvent A | 20 mM MES + | 20 mM | 10 mM | 20 mM MES + | 20 mM | 20 mM |
| | 1 mM Na2EDTA | ACES | NaHPO₄ | 1 mM Na2EDTA | HEPES | ACES |
| Solvent B | Buffer A + | Buffer A + | Buffer A + | Buffer A + | Buffer A + | Buffer A + |
| | 250 mM NaCl | 200 mM NaCl | 100 mM NaCl | 250 mM NaCl | 200 mM NaCl | 200 mM NaCl |
| pH | pH 6.0 | pH 7.5 | pH 7.5 | pH 6.0 | 1: pH 7.5 | pH 7.5 |
| | | | | | 2: pH 8.0 | |
| Gradient | From 18 to | From 8 to | From 15 to | From 18 to | 1: From 1 to | From 8 to |
| | 58% B in | 45% B in | 55% B in | 58% B in | 47% B in | 45% B in |
| | 65 min | 40 min | 30 min | 65 min | 40 min | 40 min |
| | | | | | 2: From 0 to | |
| | | | | | 47% B in | |
| | | | | | 40 min | |
| Column | 34° C. | 35° C. | 23° C. | 34° C. | 40° C. | 35° C. |
| temperature | | | | | | |
| Flow rate | 0.8 mL/ | 0.5 mL/ | 0.8 mL/ | 0.8 mL/ | 0.8 mL/ | 0.5 mL/ |
| | min | min | min | min | min | min |
| Injection | 50 μg | 100 μg | 50 μg | 50 μg | 100 μg | 100 μg |
| amount | protein | protein | protein | protein | protein | protein |

TABLE 8
DEVELOPMENT OF IEC PROTOCOL TO ANALYZE FDC CHARGE VARIANTS.
TEST PARAMETERS OF METHOD 6 ABOVE WERE INVESTIGATED.

| | Method 6 | Method 6A | Method 6B | Method 6C | Method 6D |
|---|---|---|---|---|---|
| Column | MabPac SCX-10 | | | | |
| Solvent A | 20 mM | 20 mM | 20 mM | 20 mM | 20 mM |
| | ACES | ACES | ACES | ACES | ACES |
| Solvent B | Buffer A + | Buffer A + | Buffer A + | Buffer A + | Buffer A + |
| | 200 mM | 200 mM | 200 mM | 200 mM | 200 mM |
| | NaCl | NaCl | NaCl | NaCl | NaCl |
| pH | pH 7.5 | pH 6.8 | pH 7.5 | pH 7.5 | pH 7.5 |
| Gradient | From 8 | From 8 to | From 3 to | From 0 to | From 1 to |
| | to 45% B | 45% B in | 40% B in | 35% B in | 47% B in |
| | in 40 min | 40 min | 30 min | 30 min | 40 min |
| Column | 35° C. | 35° C. | 35° C. | 40° C. | 40° C. |
| temperature | | | | | |
| Flow rate | 0.5 mL/ | 0.5 mL/ | 1.0 mL/ | 0.8 mL/ | 0.8 mL/ |
| | min | min | min | min | min |
| Injection | 100 μg | 100 μg | 100 μg | 100 μg | 100 μg |
| amount | protein | protein | protein | protein | protein |

Based on the HPLC parameters described in table 8 above, several experimental designs (Design of Experiment, DoE) were carried out. The following parameters were tested:
gradient profile
flow rate (0.5-1.0 mL/min)
pH value of mobile phase A and B (7.4-7.6 and 6.8)
NaCl concentration in mobile phase B (100-300 mM)
column temperature (25-40° C.)

By analyzing the data obtained in the frame of these experiment designs, the following test parameters showed the best results:
Eluent A 20 mM ACES, pH 7.5
Eluent B 20 mM ACES, 200 mM NaCl, pH 7.5
Column MabPac SCX-10, BioLC, 4×250 mm
Column temperature 40° C.
Flow rate 0.8 mL/min
Injection amount 10 μL, (100 μg protein)
Gradient from 1 to 47% B in 40 min

To analyze the robustness of this developed method, an experimental factorial design based on DoE was performed. Therefore, the following parameters were varied in a matrix:
ACES concentration: 18-22 mM
NaCL concentration: 180-220 mM
Column temperature: 36-44° C.
Flow rate: 0.7-0.9 mL/min
pH: 7.4-7.6
injection volume: 8-12 μL (80-120 μg)

The results of these experiments showed that the test method is robust within the tested range with regard to trastuzumab main peak (Peak 7) and peak 8. However, a high variability was observed among the purity values obtained for Pertuzumab Main Peak (Peak 4) and the Middle Region (region between Pertuzumab Main Peak and Trastuzumab Main Peak). This variability strongly depended of the pH and the column temperature. The statistical evaluation of this experiments resulted in the settings for the final method:
Eluent A 20 mM ACES, pH 7.6
Eluent B 20 mM ACES, 200 mM NaCl, pH 7.6
Column temperature 36° C.
Flow rate 0.8 mL/min
Injection amount 10 μL (100 μg protein)
Gradient from 1 to 47% B in 40 min

Possible alternatives for the determination of the charge heterogeneity of Pertuzumab/Trastuzumab FDC variants were evaluated. Among these alternatives, the suitability of different column types and of pH-gradient method was assessed.

Several separation attempts were also conducted using a weak anion-exchange column ProPac WAX-10 bio LC, 4×250 mm under the following chromatographic conditions:
The following eluents A and B were prepared and tested:
   1. A=20 mM CAPSO pH 10.0, B=20 mM CAPSO+250 mM NaCl, pH 10.0
   2. A=20 mM piperazine pH 10.0, B=20 mM piperazine+250 mM NaCl, pH 10.0
   3. A=20 mM trisma pH 10.5, B=20 mM trisma+250 mM NaCl, pH 10.5
   4. A=20 mM trisma pH 8.0, B=20 mM trisma+250 mM NaCl, pH 8.0
   5. A=20 mM phosphat pH 11.0, B=20 mM phosphat+250 mM NaCl, pH 11.0
Column temperature 30° C.
Flow rate 0.8 mL/mM; 1.0 mL/min
Injection amount 5 μL (50 μg protein)
Gradient 1 From 0 to 100% B in 60 min
Gradient 2 From 0 to 100% B in 40 min

With all of the conditions tested for the weak anion-exchange column, the species of interest are not retained on the column and elute together with the injection peak, thus showing that these experimental conditions are not suitable at all for the separation of the charges variants of Pertuzumab/Trastuzumab FDC.

Experiments in pH-Gradient Separation Mode

The suitability of an IEC method based on a pH-gradient was assessed as a possible alternative to a salt-gradient method. A strong cation exchange column (MabPac SCX-10 column) was used with following HPLC test parameters:
Eluent A 10 mM Tris, 10 mM Phosphat, 10 mM Piperazin, pH 6.0
Eluent B 10 mM Tris, 10 mM Phosphat, 10 mM Piperazin, pH 11.0
Eluent C 100 mM NaCl
Eluent D Pure water
Column MabPac SCX-10, BioLC, 4×250 mm
Column temperature 35° C.
Flow rate 0.5 mL/min
Injection amount 10 μL (10 μg protein)
Gradient From 10 to 50% B in 45 mM (see details below)
Equipment Waters Alliance

Eluents C and D were combined to provide a constant salt concentration of 0 mM, 10 mM, 20 mM, 30 mM, 40 mM and 50 mM NaCl, respectively. Therefore, the ratio of eluent C/eluent D was varied from 0% eluent C/50% eluent D (0 mM NaCl) to 50% eluent C/0% eluent D (50 mM NaCl). Perjeta SC (lot GB0005, c=120 mg/mL) and Herceptin SC (lot P0003, c=120 mg/mL) were used individually as well as co-mixtures to perform the experiments. Test samples were diluted with 90% eluent A/10% eluent B to a final concentration of 1 mg/mL.

The best separation was obtained using 40 mM NaCl; nevertheless, under these conditions, peaks elute very early, with the two main peaks presenting broad shapes and reduced heights.

An experimental design (DoE) was conducted with the salt concentration set at 40 mM NaCl while varying gradient profile and flow rate. Nevertheless, no statistical model could be established, showing the lack of robustness of the investigated method even by minor modifications of the test conditions.

From the experiments conducted above, the most critical parameters for the IEC method appear to be: 1. pH value, 2. column type, 3. column temperature, 4. gradient profile. These parameters have a significant impact on resolution. On the other hand, buffer type and concentration, salt concentration, flow rate and injection volume have less impact on the resolution.

### Example 10: IEC to Analyze FDC Charge Variants Purpose and Principle

IE-HPLC separates proteins present in drug product according to their charge properties in the dissolved state. This separation is based on the interaction of surface charges of the protein with charged groups present on the surface of the column packing. In cation-exchange HPLC, as used in this analytical procedure, acidic species elute first and more basic species elute later, in the salt gradient. The same method is applied for FDC drug product LD and MD. FDC MD reference standard is used for testing of both FDC drug product LD and MD.

Equipment and Materials
HPLC system equipped with a UV detector (Waters Alliance 2695/e2695 with 2487/2489 detector or equivalent)
HPLC column (Thermo Scientific MAbPac SCX-10, 4 mm-250 mm, particle size: 10 μm or equivalent)

Solutions
Drug Product Dilution Buffer: 20 mM L-Histidine/L-Histidine monohydrochloride, 105 mM trehalose, 100 mM sucrose, 10 mM methionine, 0.04% [w/v] polysorbate 20, pH 5.5±0.2
Mobile Phase A: 20 mM ACES, pH 7.60±0.05
Mobile Phase B: 20 mM ACES, 200 mM sodium chloride, pH 7.60±0.05
CpB solution: 1 mg/mL CpB (in Mobile Phase A)

Preparation of Sample Solution:

Dilute FDC drug product with mobile phase A to prepare a sample solution containing a total protein concentration of approximately 10 mg/mL and CpB of approximately 0.08 mg/mL.

Preparation of Blank Solution:

Dilute drug product dilution buffer in the same manner as the samples.

CpB Digestion

Incubate the reference standard, sample, and blank solutions for 20±5 minutes at 37° C.±2° C. Samples are stored at 10° C.±4° C. until analyzed and HPLC analysis has to be completed within 24 hours.

Procedure

Before injecting the first sample, rinse the column with 99% mobile phase A until a stable baseline is obtained. Optionally, inject reference solution for the purpose of column conditioning until a visual evaluation of the chromatograms demonstrates consistent profiles for at least two consecutive injections.

Operating Parameters
Detection wavelength: 280 nm
Injection volume: 10 μL
Flow rate: 0.8 mL/min
Column temperature: 36° C.±2° C.
Autosampler temperature: 10° C.±4° C.
Run time: 60 min

Gradient

| Time (min) | Mobile Phase A (%) | Mobile Phase B (%) |
|---|---|---|
| 0 | 99 | 1 |
| 3 | 99 | 1 |
| 43 | 53 | 47 |
| 44 | 0 | 100 |
| 50 | 0 | 100 |
| 51 | 99 | 1 |
| 60 | 99 | 1 |

Injection Protocol

The injections are performed in the following order:
1. Mobile phase A
2. Blank solution
3. Reference standard
4. Sample(s) (up to 10 samples)
5. Reference standard
6. Blank solution

Note: If more than 10 samples are to be analyzed, bracket every 10 samples with a reference standard injection.

Results:

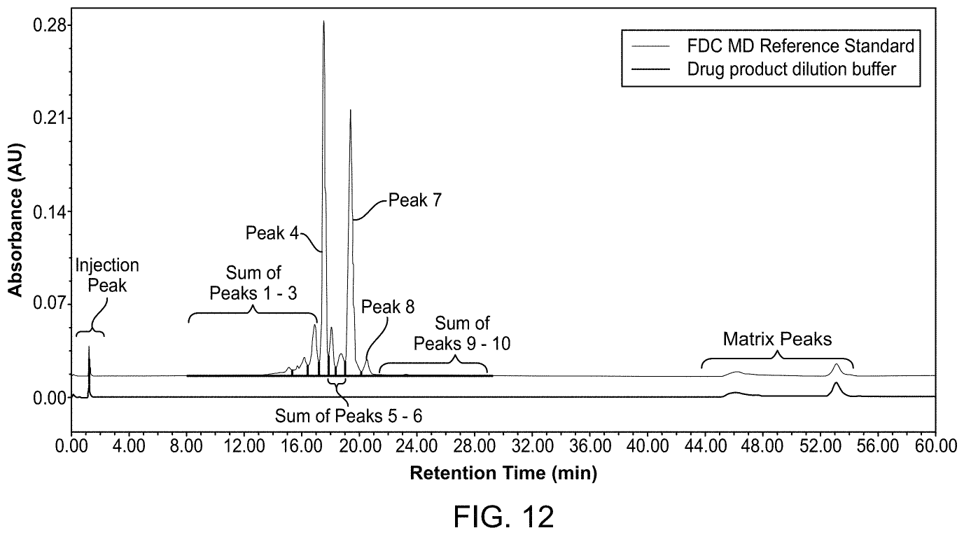

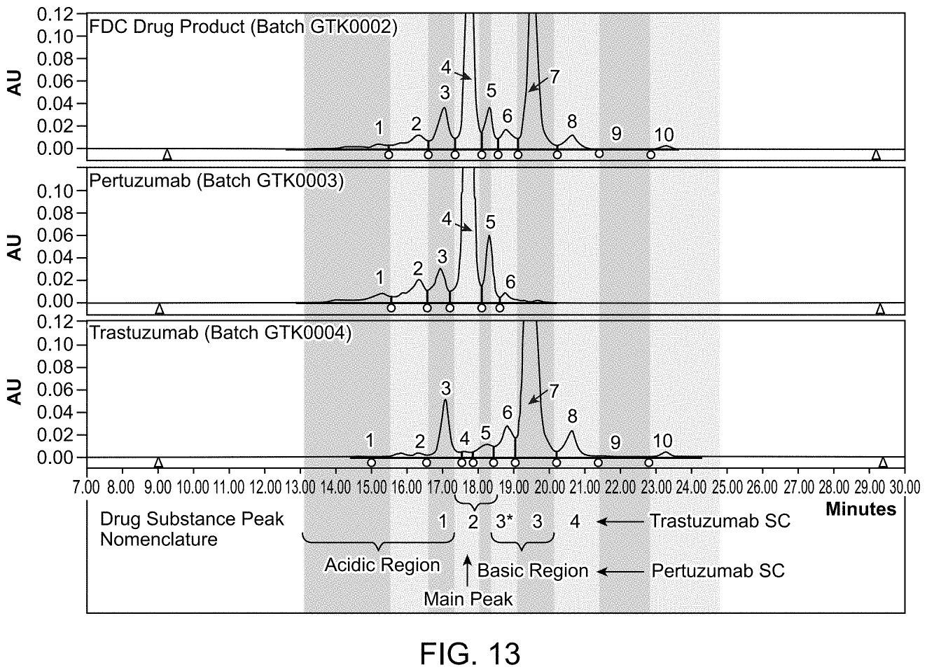

The FDC drug product IE-HPLC method has been developed and optimized to enable the best achievable separation of pertuzumab and trastuzumab charge variants. Due to the similar isoelectric points of pertuzumab (pI 8.7) and trastuzumab (pI 8.4), IE-HPLC is not able to completely separate all charge variants of the two antibody molecules (refer to FIG. 13). All critical charge variants of the individual molecules can be controlled in the FDC drug product as all relevant peaks are resolved. The reported assay parameters for FDC drug product are Sum of Peaks 1-3, Peak 4 (Main Peak Pertuzumab), Sum of Peaks 5-6, Peak 7 (Main Peak Trastuzumab), Peak 8, and Sum of Peaks 9-10. An exemplary chromatogram is shown in FIG. 12.

### Example 11: HPLC Robustness and Repeatability Studies

Various experiments were performed in order to evaluate the robustness of the analytical procedure of example 10 against different input variables. These input variables were inter alia:
Column temperature (32° C., 36° C., 40° C.)
Flow rate (0.7 mL/min, 0.8 mL/min, 0.9 mL/min)
pH of the mobile phases A & B (pH 7.5 to 7.7)
Sodium chloride concentration in mobile phase B (180 mM to 220 mM)

The profiles and results obtained upon analysis after change of the parameter of interest were compared with the profiles and results of the analysis according to the target parameters. The relative peak areas (in area %) of Peak 4, Peak 7, Sum of Peaks 1-3, and Peak 8 were used for the calculation of the relative difference between results. The results met the acceptance criteria, thus demonstrating that the procedure is suitably robust for its intended purpose.

The repeatability of the analytical procedure was demonstrated for Peak 4, Peak 7, Sum of Peaks 1-3, and Peak 8 in the range of:
50 μg to 149 μg injected protein for FDC drug product LD, covering 50% to 149% of the nominal working amount (100 μg protein)
51 μg to 153 μg injected protein for FDC drug product MD, covering 51% to 153% of the nominal working amount (100 μg protein).

### Example 12: Stability-Indicating Properties

Non-stressed and stressed FDC drug product MD and LD samples were tested with the method of example 10 and the ability of the procedure to separate, identify, and determine the purity of the antibodies under different stress conditions was demonstrated. The following stress conditions were tested: thermal stress, forced oxidation, high-pH (pH 7.4) stress, low-pH (pH 4) stress and light stress. Impurities and related substances of different charges were separated. Compared to the non-stressed sample (FIG. 12 and FIG. 13), the chromatograms of the stressed samples show increased amounts of Sum of Peaks 1-3 and Peak 8 (data not shown). As conclusion, the procedure is stability indicating.

### Example 13 Potency of Trastuzumab and Pertuzumab Charge Variants and CDR Affinity Mutants in FDC by ELISA

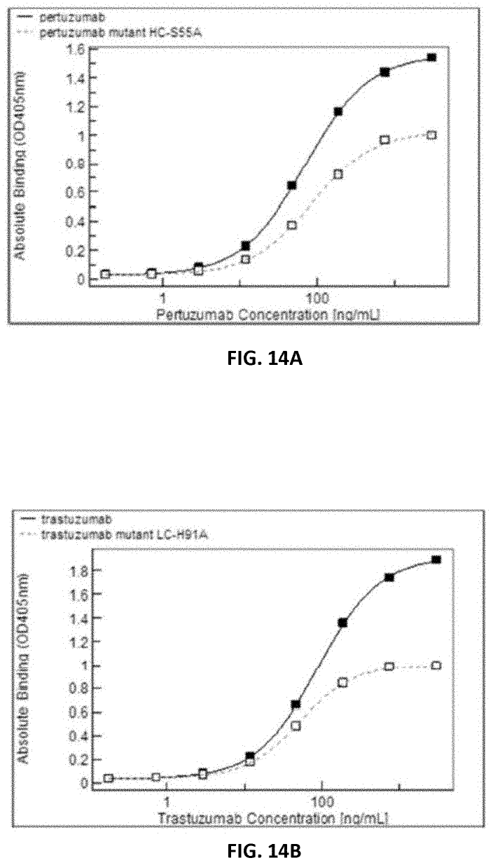

The capability of the ELISAs to reflect the anti-proliferative activity was demonstrated for charge variants and CDR affinity-mutants: Pertuzumab and trastuzumab HER2 affinity-mutants as described above were tested in the anti-proliferation assays and ELISAs (FIG. 9 and FIG. 14 respectively). The absence of dose-response curve or the shift to higher concentrations observed for the HER2 affinity-mutants, together with the failure to fulfill the similarity criteria (parallelism and higher-asymptote deviation for the anti-proliferation assays and ELISAs, respectively), demonstrates similar large reductions in potency. For the evaluation of the charge variants, supportive technical batches containing either trastuzumab or pertuzumab in the FDC drug product MD formulation buffer were used to exclude the aforementioned cross-interference of the FDC drug product in the cell-based assays. All IE-HPLC fractions showed similar potencies in both cell-based assays and ELISAs (taking into account the respective method precisions), except for Peak 9 (trastuzumab with increased Fc Met261 oxidation). Although this Fc oxidation at Met261 should not impact target binding activity of the CDRs, this variant showed reduced potency in the trastuzumab ELISA (73% vs. 91% in the cell-based assay).

It could not be completely resolved whether the fractionation process of these isoforms, which are present only in very small amounts, contributed to this finding and whether the potency values from both assays can really be considered different. However, the trastuzumab ELISA is regarded as conservative in this respect, since it would indicate a decrease in potency that is not reflected by the cell-based anti-proliferation assay.

The ELISAs are equal to the anti-proliferation assays in the ability to control the bioactivity of the product variants known to impact bioactivity, as detailed below:
The trastuzumab deamidated product variant HC Asn55/isoAsp55 and LC Asn30/Asp30 in Peak 1 showed reduced activity in both assays.
The trastuzumab product variant with succinimide at the Asp102 position in one heavy chain and increased Fc Met oxidation in Peak 10 showed reduced activity in both assays.
All other IE-HPLC fractions, including Peaks 4 and 7, corresponding to the main peaks of pertuzumab and trastuzumab, respectively, showed unchanged activity between 80% and 120% in both assays, as expected.

In addition, although similar potencies for Peak 8 were obtained in both cell-based assays and ELISAs, it is acknowledged that the known negative impact of HC IsoAsp102 on trastuzumab IV potency was not observed in this study. The isomerization of HC Asp102 to IsoAsp at one heavy chain of trastuzumab eluting in IE-HPLC Peak 4 corresponds to IE-HPLC Peak 8 in the FDC drug product. Additional studies on HC Asp102/isoAsp102 form's impact on anti-proliferative activity performed during the development of trastuzumab SC showed a less pronounced impact for trastuzumab SC than for trastuzumab IV.

This may be attributed to the optimization of the formulation (e.g., pH change) and the increased stability of trastuzumab SC. Finally, it is noted that the control of this variant is maintained for the FDC drug product through defined acceptance criteria by IE-HPLC.

TABLE 9
CORRELATION OF THE BINDING AND ANTI-PROLIFERATIVE
ACTIVITIES OF TRASTUZUMAB AND PERTUZUMAB

| | Pertuzumab in FDC Drug Product MD | | | |
|---|---|---|---|---|
| | Formulation Buffer | | Trastuzumab in FDC Drug Product MD | |
| | (Batch GTK0003) | | Formulation Buffer | |
| | | Relative Potency | (Batch GTK0004) | |
| | | of Pertuzumab by | | Relative Potency |
| | Relative Potency | MDA-MB-175 VII | | of Trastuzumab |
| | of Pertuzumab by | Ant-Proliferation | Relative Potency | by BT-474 |
| Sample | ELISA (%) | Assay (%) | of Trastuzumab | Anti-Proliferation |
| IE-HPLC Fractions (non-stressed)^{a} | | | by ELISA (%) | Assay (%) |
| Peak 1 | 88 | Equipotent^{b} | 75 | 61 |
| Peak 2 | 95 | 84 | 87 | 90 |
| Peak 3 | 93 | 112 | 95 | 114 |
| Peak 4 | 113 | 107 | 86 | 83 |
| Peak 5 | 109 | 109 | 89 | 109 |
| Peak 8 | 81 | Equipotent^{b} | 102 | 116 |
| Peak 7 | NA^{c} | NA^{c} | 101 | 109 |
| Peak 8 | NA^{c} | NA^{c} | 100 | 105 |
| Peak 9 | NA^{c} | NA^{c} | 73 | 91 |
| Peak 10 | NA^{c} | NA^{c} | 71 | 73 |

^{a}For characterization of fractions, refer to Example 14.
^{b}Qualitative estimates provided relative to the reference standard as dose-response curves of sample and reference standard are not similar and therefore relative potency is not reportable (n ≥ 3 single plate results).
^{c}FDC drug product IE-HPLC Peaks 7 to 10 contain only trastuzumab isoforms.

### Example 14: Characterization of Charge Variants

Charge variants of the FDC drug product separated and isolated by the FDC drug product IE-HPLC method were characterized (FIG. 13). In addition, charge variants of the individual antibodies in the FDC formulation at the time of release were isolated by the same IE-HPLC method and characterized (FIG. 13).

A comprehensive peak characterization study using the following methods was performed to confirm the charge variants of FDC drug product:
LC-MS/MS of tryptic antibody peptides for the assessment of chemical degradation sites.
Boronate affinity chromatography for the evaluation of lysine glycation content
2-AB labeling combined with HILIC for the analysis of Fc glycosylation

LC-MS Peptide Mapping:

LC-MS/MS peptide mapping and quantitation of relevant amino acid modifications was conducted as described by Schmid et al. 2018 (Schmid I, Bonnington L, Gerl M, et al. Assessment of susceptible chemical modification sites of trastuzumab and endogenous human immunoglobulins at physiological conditions. Commun Biol 2018; 1:28). In brief, all samples were denatured with 8 mol/L guanidine hydrochloride (pH 6.0) and reduced with dithiothreitol at 50° C. for 1 h. Samples were buffer-exchanged (0.02 mol/L histidine-hydrochloride, pH 6.0) and further digested with trypsin at 37° C. for 18 h. Peptide separation on a BEH C18 column was performed on an ACQUITY UPLC system. Online mass spectrometric detection was accomplished with a Synapt G2 HDMS Q-ToF mass spectrometer. For relative quantitation of modified peptides, GRAMS AI software was used.

Boronate Affinity Chromatography:

The boronate affinity chromatography was carried out using a TSKgel Boronate-5PW affinity column. An elution buffer consisting of 100 mmol/L Hepes, 70 mmol/L Tris, 200 mmol/L NaCl, 500 mmol/L sorbitol (pH 8.6) was used for chromatographic separation on an HPLC system equipped with UV detection at 280 nm. Peak integration and glycation quantitation was performed as described (Fischer S, Hoernschemeyer J, Mahler H C. Glycation during storage and administration of monoclonal antibody formulations. Eur J Pharm Biopharm. 2008; 70:42-50).

Glycan Analysis:

For the assessment of Fc glycosylation, samples were buffer-exchanged with ammonium formate buffer (pH 8.6) and incubated with PNGase F at 45° C. for 1 h. Glycan 2-AB labeling was performed at 65° C. for 2 h. Labeled glycan structures were HILIC-separated and fluorescence-detected for subsequent peak integration and glycan quantitation as described (Reusch D, Haberger M, Maier B, et al. Comparison of methods for the analysis of therapeutic immunoglobulin G Fc-glycosylation profiles—part 1: separation-based methods. MAbs. 2015; 7:167-79.)

Results and Conclusion:

All charge variants (≥1% relative abundance) found for the individual pertuzumab and trastuzumab molecules in the FDC formulation were also detected in FDC drug product. No new charge variants were detected in the FDC drug product compared to the individual antibodies in the FDC formulations at time of release and after storage. Table 10 summarizes the findings.

TABLE 10
IE-HPLC PEAK CHARACTERIZATION RESULTS OF FDC DRUG PRODUCT

| Sample | IE-HPLC Peak 1 | IE-HPLC Peak 2 | IE-HPLC Peak 3 | IE-HPLC Peak 4 | IE-HPLC Peak 5 |
|---|---|---|---|---|---|
| Pertuzumab | Deamidation of | Deamidation of | Deamidation of | Deamidation of | Lys glycation |
| trastuzumab | HC-Asn-55 and | HC-Asn-55 and | LC-Asn-30 | LC-Asn-30 and | (trastuzumab); |
| FDC MD | LC-Asn-30 | LC-Asn-30 | (trastuzumab); | isomerization of | N-terminal VHS |
| (Batch | (trastuzumab); | (trastuzumab); | Deamidation of | HC-Asp-102 | and |
| GTK0002) | Lys glycation | Fc sialic acid and | HC-Asn-391 and | (trastuzumab); | pyroglutamate on |
| | (pertuzumab) | Lys glycation | HC-Asn-327 | IE-HPLC | heavy and light |
| | | (pertuzumab) | (trace levels), | main peak | chain |
| | | | Fc sialic acid and | (pertuzumab) | (pertuzumab) |
| | | | Lys glycation | | |
| | | | (pertuzumab) | | |
| Pertuzumab in | Lys glycation | Fc sialic acid and | Low levels of | IE-HPLC | N-terminal VHS |
| FDC drug | | Lys glycation | HC-Asn-391 and | main peak | and |
| product MD | | | HC-Asn-327 | | pyroglutamate on |
| formulation | | | deamidation, | | heavy and light |
| | | | Fc sialic acid and | | chain |
| | | | Lys glycation | | |
| Trastuzumab | Deamidation of | Deamidation of | Deamidation of | Deamidation of | Lys glycation |
| in FDC drug | HC-Asn-55 and | HC-Asn-55 and | LC-Asn-30 | LC-Asn-30 and | |
| product MD | LC-Asn-30 | LC-Asn-30 | | isomerization of | |
| formulation | | | | HC-Asp-102 | |
| Sample | IE-HPLC Peak 6 | IE-HPLC Peak 7 | IE-HPLC Peak 8 | IE-HPLC Peak 9 | IE-HPLC Peak 10 |
| Pertuzumab | Deamidation of | IE-HPLC main | Isomerization of | Increased Fc | Isomerization of |
| trastuzumab | HC-Asn-392 and | peak | HC-Asp-102 | oxidation | HC-Asp-102 and |
| FDC MD | HC-Asn-328 | (trastuzumab) | (trastuzumab) | (trastuzumab) | increased Fc |
| (Batch | (trace levels), Fc | | | | oxidation |
| GTK0002) | sialic acid and | | | | (trastuzumab) |
| | Lys glycation | | | | |
| | (trastuzumab); | | | | |
| | N-terminal VHS | | | | |
| | light chain and C- | | | | |
| | terminal lysine | | | | |
| | and proline amide | | | | |
| | at heavy chain | | | | |
| | (pertuzumab) | | | | |
| Pertuzumab in | N-terminal VHS | NA | NA | NA | NA |
| FDC drug | light chain and C- | | | | |
| product MD | terminal lysine | | | | |
| formulation | and proline amide | | | | |
| | at heavy chain | | | | |
| Trastuzumab | Deamidation of | IE-HPLC main | Isomerization of | Increased Fc | Isomerization of |
| in FDC drug | HC-Asn-392 and | peak | HC-Asp-102 | oxidation | HC-Asp-102 and |
| product MD | HC-Asn-328 | (trastuzumab) | (trastuzumab) | (trastuzumab) | increased Fc |
| formulation | (trace levels), Fc | | | | oxidation |
| | sialic acid and | | | | (trastuzumab) |
| | Lys glycation | | | | |

Sum of Peaks 1-3 contains the acidic variants of pertuzumab (deamidation of HC-Asn-391, FC sialic acid, and lysine glycation) and trastuzumab (deamidation of LC-Asn-30 and HC-Asn-55).

Peak 4 contains pertuzumab main charge variant (i.e. native antibody) and low amounts of acidic trastuzumab variants (deamidation of LC-Asn-30 and isomerization of HC-Asp-102).

Sum of Peaks 5-6 contains basic variants of pertuzumab (N-Terminal VHS on heavy and light chains and C-terminal lysine at the heavy chain) and acidic variants of trastuzumab (deamidation of HC-Asn-392, lysine glycation, and increased Fc sialic acid content).

Peak 7 contains the main charge variant of trastuzumab (i.e. native antibody), shows no overlap with pertuzumab variants.

Peak 8 contains trastuzumab with single isomerization of HC-Asp-102 to iso-aspartic acid (at one heavy chain), shows no overlap with pertuzumab charge variants.

Sum of Peaks 9-10 contains trastuzumab charge variants with increased FC oxidation (at HC-Met-255 and -431) and isomerization of HC-Asp-102, shows no overlap with pertuzumab variants.

All abundant charge variants found in pertuzumab SC drug substance and trastuzumab SC drug substance were also detected in FDC drug product. No new charge variants were detected in the FDC drug product material at time of release and after storage. All critical charge variants of the individual molecules can be controlled in the FDC drug product.

No additional co-elutions or increase in existing peak co-elutions are observed or expected during stability because pertuzumab and trastuzumab stress-induced charge variants shift towards earlier and later elution times, respectively: The peak pattern of stressed pertuzumab shifts toward the acidic region of the chromatogram, whereas the peak pattern of stressed trastuzumab shifts toward the basic region.

### Example 15: FDC Compositions

Sum of Peaks 1-3 of FDC drug product by IE-HPLC

Sum of Peaks 1-3 of FDC drug product is composed of the following variants:
The acidic variants of pertuzumab (deamidation of HC Asn391, Fc sialic acid, and lysine glycation).
The deamidation of Asn327 in pertuzumab, which was observed in FDC drug product IE-HPLC peak characterization studies only at trace level.
The acidic variants of trastuzumab (predominantly deamidation of LC Asn30, and HC Asn55). Compared to LC Asn30, a low degradation susceptibility for HC Asn55 was verified at bioprocess and physiological conditions (Schmid I, Bonnington L, Gerl M, et al. Assessment of susceptible chemical modification sites of trastuzumab and endogenous human immunoglobulins at physiological conditions. Commun Biol 2018; 1:28).

The FDC drug product end-of-shelf-life acceptance criteria are justified based on the clinical experience and anticipated impact on PK/bioactivity and safety/immunogenicity profile. The proposed acceptance criteria are suitable to control product quality and cover potential impact of the drug substance and drug product processes and storage.

For the FDC drug product the following end-of-shelf-life acceptance criteria were established: Sum of Peaks 1-3: ≤23.0 area % (LD)/≤21.0 area % (MD). These acceptance criteria were established based on clinical experience and the assumed impact on bioactivity/PK and safety/immunogenicity profile. The extension beyond current clinical experience is considered justified by a low impact on bioactivity and PK and no risk to immunogenicity/safety.

Safety and immunogenicity considerations: Because acidic variants found in pertuzumab and trastuzumab materials are modifications commonly found in IgG antibodies, any increased levels of acidic variants within the acceptance criterion are not expected to represent new forms and thus not expected to increase risk of toxicities and ADA incidence. This is supported by the low ADA incidence in FDC drug product clinical studies and the good safety profile. Aged clinical study material with up to 18.7 area % for FDC drug product LD and 16.0 area % for FDC drug product MD was administered to patients during the pivotal study. No new acidic variants are generated during storage or handling. Moreover, it was published for trastuzumab that degradation of solvent-accessible residues located in the conserved Fc (deamidation of HC Asn387, Asn392, and Asn393) and also in the CDR (predominantly deamidation of LC Asn30 and isomerization of HC Asp102) and generally occurs significantly faster in vivo (within days) compared to bio-process and real-time storage conditions (Schmid et al. 2018). The degradation levels of the same Fc Asn deamidation sites in endogenous human antibodies were significantly higher than those observed for the liquid drug product formulation stored at 5° C. It is therefore concluded that these degradations are not posing an increased safety/immunogenicity risk to the patient (Liu Y D, van Enk J Z, Flynn G C. Human antibody Fc deamidation in vivo. Biologicals 2009; 37:313-22). This conclusion can be applied for pertuzumab deamidation as well for which only one deamidation site is detected in this peak region, located in the Fc portion (HC Asn391).

In sum, the potential 4.3 area % increase in Sum of Peaks 1-3 above levels to which patients were exposed is not expected to change the immunogenicity and safety profile of the product.

Bioactivity considerations: Relative to the maximum clinical experience at 18.7 area % (LD) and 16.5 area % (MD) for the acidic variants of pertuzumab and trastuzumab (Sum of Peaks 1-3), the specification limit of 23.0 area % (LD) and 21.0 area % (MD) could lead to a decrease by up to approximately 4% in pertuzumab and trastuzumab binding activity (according to the Potency by ELISA values described in Table 8). A 4% change in bioactivity is not considered to be impactful. Therefore, efficacy is expected to be maintained if Sum of Peaks 1-3 is present at the specification limit.

PK considerations: The antibody Fc is involved in clearance (Jefferis R. Antibody therapeutics: isotype and glycoform selection. Expert Opin Biol Ther 2007; 7:1401-13); therefore, deamidation in CDRs is not expected to impact PK. Although charge properties have been known to impact the PK behavior of an antibody, single negative charges introduced by deamidation should not impact the PK (Khawli et al. 2010). Notably, only low-level alterations in Fc deamidation (IE-HPLC Peak 3: pertuzumab HC Asn391; IE-HPLC Peak 6: trastuzumab HC Asn392) have been observed during FDC drug product stability. Therefore, PK is not expected to be impacted if Sum of Peaks 1-3 is present at the specification limit.

Peak 4 of FDC Drug Product by IE-HPLC

Peak 4 of FDC drug product is part of the reported assay parameter of the IE-HPLC method and constitutes the desired main charge isoform of pertuzumab. Its inclusion on the specifications ensures consistent purity of the product.

The acceptance criteria for drug substance and drug product release and stability testing were set in relation to the other reported assay parameters by IE-HPLC and with consideration for the manufacturing experience and stability effects. The FDC drug product acceptance criteria of ≥38 area % (LD) and >28 area % (MD) at the end of shelf life ensure the purity of the product and adequate control for the manufacturing process.

Sum of Peaks 5-6 of FDC Drug Product by IE-HPLC

Sum of Peaks 5-6 of FDC drug product is composed of the following variants:
The basic variants of pertuzumab (N-terminal VHS on heavy and light chain, N-terminal pyroglutamate, and C-terminal lysine and proline amide at heavy chain)
Acidic variants of trastuzumab (deamidation of HC Asn392, lysine glycation, and increased FC sialic acid content)

Sum of Peaks 5-6 is not controlled at FDC drug product release or stability testing as historical data have shown that the basic variants of pertuzumab and these acidic variants of trastuzumab remain unchanged during drug product manufacturing and storage and therefore are not considered to be stability-indicating parameters.

Peak 7 of FDC Drug Product by IE-HPLC

Peak 7 of FDC drug product is part of the output of the IE-HPLC method and constitutes the desired main charge isoform of trastuzumab. Its specification ensures consistent purity of the product. The acceptance criteria for drug product release and stability testing were set in relation to the other reported assay parameters by IE-HPLC and considering the manufacturing experience and stability effects. The FDC drug product acceptance criteria of ≥16.0 area % (LD) and >23.0 area % (MD) at the end of shelf life ensure the quality of the product and adequate control for the manufacturing process.

Peak 8 of FDC Drug Product by IE-HPLC

Peak 8 of FDC drug product is composed of trastuzumab with singly isomerization of HC Asp102 to iso-aspartic acid (at one heavy chain) and shows no co-elution with pertuzumab charge variants.

Peak 8 will be controlled at FDC drug product release and stability testing.

The FDC drug product end-of-shelf-life acceptance criterion≤9.0 area % (LD)/≤12.0 area % (MD) is justified based on the clinical experience and anticipated impact on PK/bioactivity and safety/immunogenicity profile. The proposed acceptance criteria are suitable to control product quality and cover potential impact of the drug substance and drug product processes and storage.

Safety and immunogenicity considerations: Because acidic variants found in trastuzumab materials are modifications commonly found in IgG antibodies, any increased levels of acidic variants with the acceptance criterion are unlikely to represent new forms and are unlikely to increase risk of toxicities and ADA incidence. FDC drug product was generally safe and well tolerated. The safety profile was comparable to the safety profile of pertuzumab IV+trastuzumab IV (P+H IV). The incidence of ADAs was low (≤5%) and without clinical consequences with respect to PK, efficacy, or safety. Aged clinical study material with up to 6.4 area % of Peak 8 for FDC drug product LD and 9.4 area % of Peak 8 for FDC drug product MD was administered to patients during the pivotal study. No new charge variants are generated during storage or handling. Moreover, it was published for trastuzumab that degradation of solvent-accessible residues located in the conserved Fc (deamidation of HC Asn387, Asn392, and Asn393) and also in the CDR (predominantly deamidation of LC Asn30 and isomerization of HC Asp102) and generally occurs significantly faster in vivo (within days) compared to bio-process and real-time storage conditions (Schmid et al. 2018). The degradation levels of the same Fc Asn deamidation sites in endogenous human antibodies were significantly higher than those observed for the liquid drug product formulation stored at 5° C. It is therefore concluded that these degradations are not posing an increased safety/immunogenicity risk to the patient (Liu et al. 2009). The potential 2.5 area % increase in Peak 8 (Isomerization HC Asp102 to iso-aspartic acid) above levels to which patients were exposed is not expected to change the immunogenicity profile of the product.

Bioactivity considerations: The enriched Peak 8 (92% peak purity, which contains mainly the single isomerization of HC Asp102 to iso-aspartic acid at one heavy chain) has similar trastuzumab activity (100% binding activity) when compared to the reference standard. Therefore, efficacy of the FDC drug product is expected to be maintained if Peak 8 is present at the specification limit.

PK considerations: Aspartate isomerization to iso-aspartic acid in the CDR of pertuzumab and trastuzumab does not alter the charge and is not expected to impact PK. Therefore, the single aspartate isomerization of trastuzumab HC Asp102 should not impact the PK.

Sum of Peaks 9-10 of FDC Drug Product by IE-HPLC

Sum of Peaks 9-10 of FDC drug product is composed of trastuzumab with single isomerization of HC Asp102 to succinimide (at one heavy chain) and shows no overlap with pertuzumab charge variants. In addition, low levels of trastuzumab Fc oxidation are detected in these peaks. Due to the low levels, no impact is expected. As the succinimide (Sum of Peaks 9-10) is in equilibrium with Peak 8 (isoAsp) and Peak 7 (Asp), it is controlled indirectly via the acceptance criteria for Peak 8 and Peak 7. Therefore, no acceptance criterion is required for Sum of Peaks 9-10 in the control system.

### Example 16: Production of FDC Compositions

Pertuzumab SC drug substance is transferred from the drug substance storage container into a steam-sterilized stainless-steel compounding vessel. Multiple pertuzumab SC drug substance batches may be combined for drug product manufacturing.

Based on the amount of the pertuzumab added to the compounding vessel (determined by the pertuzumab SC drug substance mass transferred, the density, and the pertuzumab content), the target amount of trastuzumab is defined (e.g., 1:1 API ratio for the maintenance dose). The trastuzumab SC drug substance is then added (based on the density and the trastuzumab content) to the compounding vessel. Multiple trastuzumab SC drug substance batches may be combined for FDC drug product manufacturing.

Based on the volume (determined by the mass and density) of the pertuzumab SC and trastuzumab SC drug substances added to the compounding vessel, the required amount of thawed rHuPH20 is added to the compounding vessel (based on the rHuPH20 solution content and activity). Multiple rHuPH20 batches may be combined for drug product manufacturing.

After all components are transferred to the compounding vessel the solution is then homogenized by mixing.

### Example 17: Development of RP-UPHLC Assay to Determine Content of FDC Equipment

Equivalent instrumentation and appropriate operating conditions may be used.
HPLC system: HPLC System (with in-line vacuum degasser) equipped with data acquisition software
Detector: UV/Visible Absorbance Detector or Photodiode Array Detector
Membrane filter: 0.2 μm filter (e.g. Corning Cat no. 430049)
Column. TSK-Gel G3000SWXL, 7.8×300 mm, 5 μm (Tosoh Bioscience, Cat. no. 08541) or BioSuite 250, 7.8×300 mm, 5 μm (Waters, Cat. no. 186002165)

Reagents
Purified water (Water treated with Milli-Q)
Trifluoroacetic acid (TFA) (Fluka, Cat. Nr. 40967)
Acetonitrile (Merck, Cat. Nr. 1.00030.2500)
L-Histidine, anhydrous (Sigma, Cat. Nr. H8000)
Sucrose (Merck, Cat. Nr. 1.07687)
L-Methionine (Sigma, Cat. Nr. 64319)
Glacial Acetic Acid (Merck. Cat. Nr. 1.00063.1000)
Polysorbate 20 (Sigma, Cat. Nr. 93773)
Solvent A: 0.1% TFA in Milli-Q water
Solvent B: 0.1% TFA in acetonitrile
Formulation Buffer: 20 mM Histidine-acetate, 240 mM Sucrose, 10 mM Methionine and Polysorbate 20, 0.02% [w/v], pH 5.7±0.2
Dilution buffer: 20 mM Histidine-acetate pH5.5
Column Storage Solution: 60% Acetonitrile (v/v)
Sample Solution: Dilute sample to approx. 10 mg/mL with formulation buffer. Dilute the 10 mg/mL test sample solution to approximately 1 mg/mL with dilution buffer.
Blank: Formulation buffer and dilution buffer will be injected undiluted.
Flow rate: 0.4 mL/min
Maximum pressure: 400 bar/6000 psi
Wavelength: 280 nm
Run time: 29 min
Column temperature setting: 60° C.
Auto sampler temperature setting: ≤10° C.
Injection amount: Sample and reference standard: 25 μg protein (nominal) Blank and mobile phase: same injection volume as reference standard

Gradient

| Time (minutes) | Solvent A (9%) | Solvent B (%) |
|---|---|---|
| 0.0 | 64 | 36 |
| 2.0 | 64 | 36 |
| 20.0 | 40 | 60 |
| 20.5 | 5 | 95 |
| 21.0 | 5 | 95 |
| 22.0 | 64 | 36 |
| 29.0 | 64 | 36 |

While the peaks of Pertuzumab and Trastuzumab were clearly separated with this method, a major carryover problem of the method above became apparent. After 5 injections of blank samples (formulation buffer), traces of Herceptin/Perjeta were still detectable. Therefore further method development was required. Different chromatographic techniques were tested and reverse-phase chromatography (RPC) chosen as the most suitable method for protein content analysis. Multiple parameters were evaluated with regard to method accuracy and repeatability.

Influence of Column Type on the Separation

Different types of columns were tested for Pertuzumab/Trastuzumab FDC.

TABLE 11
COLUMNS TESTED FOR RP-UHPLC PROTEIN CONTENT
METHOD, THEIR RESPECTIVE TEMPERATURES TESTED

| Column | Column temperature [° C.] | Resolution |
|---|---|---|
| BEH300 C4 | 70 | −− |
| | 80 | + |
| | 90 | ++ |
| Agilent AdvanceBio RP | 70 | −− |
| mAB Diphenyl | 80 | + |
| | 90 | ++ |
| ZXorbax 300SB C3 | 70 | + |
| | 80 | ++ |
| Poroshell 300 SB-C8 | 70 | −− |
| | 80 | − |
| | 90 | − |
| Hamilton PRP | 70/80/90 | −−− |
| Baker Wide Pore C4 | 70/80/90 | −− |
| Baker Wide Pore C18 | 70/80 | −−− |
| Grace Aquapore RP-300 | 70/80/90 | − |
| Agilent PLRP-S 300 | 80/90 | + |
| Agilent Zorbax RRHD 300- | 70 | +++ |
| Diphenyl | 80 | ++ |
| | 90 | ++ |

Several potential columns for Pertuzumab/Trastuzumab FDC were found. For example, BEH300 C4 showed a good separation but required a high column temperature (90° C.). Agilent AdvanceBio RP mAb had a similar separation as Agilent Zorbax RRHD 300-Diphenyl but overall a lower resolution. The most suitable column was determined to be the Agilent Zorbax RRHD 300-Diphenyl, 2.1×100 mm column, which exhibited a low carry-over and improved the separation of the two antibodies compared to the initial method.

DoE (Design of Experiment) for Agilent Zorbax RRHD 300-Diphenyl Column

Mobile phases, flow-rates, gradients and column compartment temperatures were tested on the Agilent Zorbax RRHD 300-Diphenyl, 2.1×100 mm column A DoE for the development of reversed-phase protein content method was set up using MODDE®. A summary of the factors tested within the scope of the DoE is listed in Table 12.

TABLE 12
VARYING FACTORS DETERMINED FOR DOE SCREENING.

| Name | Abbreviation | Unit | Type | Setting | Precision | |
|---|---|---|---|---|---|---|
| Flow | Flow | mL/min | Quantitative | 0.6 to 0.8 mL/min | 0.005 | mL/min |
| Temp | Temp | ° C. | Quantitative | 70 to 90° C. | 0.5° | C. |
| Hold Time | Hold | Min | Quantitative | 0 to 3 min | 0.05 | min |
| Gradient | Grad | Min | Quantitative | 10 to 20 min | 0.3 | min |

| | | | | | |
|---|---|---|---|---|---|
| Start | Start | % B | Quantitative | 20 to 30% B | 0.05% B |

Evaluation of ‘Resolution for Trastuzumab/Pertuzumab’:

Overall, resolution of the reversed-phase chromatography method for protein content determination was strongly influenced by the flow-rate and gradient length. A lower flow-rate and longer gradient length resulted in an improved resolution. Column compartment temperature and the starting condition had a weaker but not insignificant influence on the method. A temperature of 70° C. and a relatively high starting condition of 30% B proved to produce the best results. Adding additional hold time had no effect on the resolution.

Evaluation of ‘Sum of Minor Forms’:

The sum of minor forms strongly depended on the starting concentration (high) and the column temperature (low). Flow rate and gradient time only had minor influences. The hold time alone was insignificant but showed an effect once combined with flow rate and column temperature.

Evaluation of ‘Height Ratio Trastuzumab’:

To achieve a high height ratio, i.e. no additional shoulder for Trastuzumab main peak, the temperature had to be lowered. Flow rate analysis was ambiguous. Gradient time and starting condition should ideally be in the higher range. Again, additional hold time showed no effect.

Evaluation of ‘USP Tailing Pertuzumab’:

To reduce tailing of the Pertuzumab main peak the flow rate should be increased and the gradient as well as the starting condition decreased. Again, additional hold time showed no effect.

According to the DoE results, the following parameters were chosen:
Flow rate 0.8 mL/min
Wavelength 280 nm
Column temperature 70° C.
Autosampler temperature 10° C.
Run time 20 min

TABLE 13
DOE GRADIENT

| Time [min] | % B |
|---|---|
| 0 | 30 |
| 20 | 50 |
| 25 | 90 |
| 26 | 30 |
| 30 | 30 |

Based on these results, column temperature, gradient and flow rate were further optimized.

Influence of Column Temperature on the Separation

An elevated temperature in reversed-phase chromatography can have a significant effect on peak separation, tailing effects and system pressure. Three different temperatures were chosen to be tested on the Agilent Zorbax RRHD 300 Diphenyl column. The temperature testing was performed within the scope of the DoE (results not shown). Overall, the retention times shifted towards an earlier elution with increasing column compartment temperatures. This was expected as the viscosity of the eluents and secondary column interactions are decreased with increasing temperature. However, with increasing column compartment temperature the overall resolution decreased. Therefore, the most suitable column compartment temperature within the scope of the experiment was 70° C.

Influence of Gradient Profile on the Separation

Gradients have a drastic influence on the separation of analytes. For protein content determination by reversed-phase chromatography, four major gradients were tested (see Table 14) on the Agilent Zorbax RRHD 300 Diphenyl column. For a direct gradient comparison, column compartment temperature was constantly set to 70° C. and flow-rate to 0.6 mL/min. Once the final flow rate had been set, a re-assessment of gradients had to be done. The final gradient for the RP protein content method is listed in Table 14, Gradient 5. The initial DoE gradient (Table 13) had been altered for optimal separation and equilibration time with the new flow rate (0.3 mL/min).

TABLE 14
PROFILES OF FIVE GRADIENTS SCREENED. GRADIENTS
1-4 WERE SCREENED WITH A FLOW RATE OF
0.6 ML/MIN, WHEREAS GRADIENT 5 WITH HALF
THE FLOW RATE (0.3 ML/MIN).

| Gradient 1 | | Gradient 2 | | Gradient 3 | | Gradient 4 | | Gradient 5 | |
|---|---|---|---|---|---|---|---|---|---|
| Time | | Time | | Time | | Time | | Time | |
| [min] | % B | [min] | % B | [min] | % B | [min] | % B | [min] | % B |
| 0 | 30 | 0 | 20 | 0 | 20 | 0 | 30 | 0 | 30 |
| 10 | 50 | 3 | 20 | 20 | 50 | 3 | 30 | 15 | 45 |
| 15 | 90 | 13 | 50 | 25 | 90 | 23 | 50 | 20 | 90 |
| 16 | 30 | 18 | 90 | 26 | 20 | 28 | 90 | 21 | 30 |
| 21 | 30 | 19 | 20 | 31 | 20 | 29 | 30 | 30 | 30 |
| | | 24 | 20 | | | 34 | 30 | | |

Observations:

All five gradients tested showed sufficient protein retention and a well-chosen starting condition ranging from 20-30% B. Any starting condition within this range would be suitable for Pertuzumab/Trastuzumab FDC separation. However, to shorten gradient and run time, a 30% starting condition was chosen. Considering gradient time, the range tested (10-20 min) was well-chosen. As the gradient time was also heavily influenced by the flow-rate, a separation time of 15 min at a flow rate of 0.3 mL/min was chosen eventually.

With a 10-minute separation time, in particular with a gradient steepness of 30% B, both antibodies eluted within a window of only 1-2 minutes. However, a 20-minute separation and a gradient steepness of 20% B resulted in a broader elution profile and a less intense detector signal.

A final 15 min separation time was combined with a gradient steepness of 15% B. Together with a slow flow rate (0.3 mL/min) it showed a good baseline separation of both antibodies without losing too much signal intensity.

Influence of Flow-Rate on the Separation

Eventually the most suitable flow rate had to be determined. A faster flow rate usually means earlier elution but might lead to a loss in resolution. Initial experiments were performed with a flow rate of 0.6 or 0.8 mL/min. It was later discovered, that a lower flow rate is more beneficial for this particular RP protein content method. Four different flow rates were tested (0.3 mL/min to 0.6 mL/min) on the Agilent Zorbax RRHD 300 Diphenyl column using single mAb containing samples. For a direct comparison, column compartment temperature was constantly set to 70° C. and the gradient listed in Table 13 was used for all separations. Decreasing the flow rate resulted in more narrow peak shapes and higher signal intensities. Retention times were shifted towards a later elution. The resolution, in particular for side peaks, improved with a lower flow rate. Hence for this method, a flow rate of 0.3 mL/min is ideal. The gradient runtime was set to 30 min and showed sufficient column re-equilibration at 0.3 mL/min. Based on these experiments, it was found that the most critical parameters for this method are column type, column temperature and flow rate. Using a phenyl-based column resulted in improved resolution and no carry-over issues. Temperatures of 64° C.-76° C. and 66° C.-74° C. were tested and had no significant impact on method performance. In the scope of the robustness experiments of phase III and BLA/MAA method validation, flow rates of 0.4 and 0.2 mL/min were tested and found to not have a significant impact on method performance.

### Example 18: RP-UPHLC Assay to Determine Content of FDC

Note: Equivalent instrumentation; appropriate operating conditions; and solvents, chemicals, and reagents of equivalent quality may be used.

The content of pertuzumab and trastuzumab in FDC drug product is determined by RP-UHPLC with UV detection. Pertuzumab and trastuzumab are separated based on differences in their hydrophobicity. The respective contents of pertuzumab and trastuzumab are calculated from an external calibration curve generated in each sequence of analysis by injecting varying volumes of FDC reference standard. The same method is applied for FDC drug product LD and MD. Each dosage form is measured against the corresponding reference standard.

Equipment and Materials
UHPLC system equipped with a UV detector (Thermo Ultimate 3000 RS or equivalent)
UHPLC column (Agilent Zorbax RRHD 300-Diphenyl, 2.1 mm×100 mm, particle size: 1.8 μm or equivalent)

Reagents
2-Propanol
Acetonitril
TFA
L-histidine anhydrous
L-histidine monohydrochloride monohydrate
Sucrose
Trehalose
L-Methionine
Polysorbate 20
Sodium hydroxide
Hydrochloric acid
Purified water (e.g., MilliQ)

Solutions

Drug Product Dilution Buffer

20 mM L-Histidine/L-Histidine monohydrochloride, 105 mM trehalose, 100 mM sucrose,

10 mM methionine, 0.04% (w/v) polysorbate 20, pH 5.5±0.2

Mobile Phase A

2% (v/v) 2-propanol, 0.1% (v/v) TFA in water

Mobile Phase B

70% (v/v) 2-propanol, 20% (v/v) Acetonitrile, 10% (v/v) Mobile Phase A

Preparation of Reference Standard Solutions

Note: For measuring FDC drug product LD and MD samples, FDC LD reference standard and FDC MD reference standard have to be prepared, respectively. The respective reference solution must be prepared in duplicate (Reference A and Reference B solutions). Dilute the respective reference standard to a total protein concentration of 1 mg/mL using drug product dilution buffer.

Preparation of Sample Solution

Dilute FDC drug product with drug product dilution buffer to prepare a sample solution containing a total protein concentration of 1 mg/mL.

Procedure

Before injecting the first sample, rinse the column with 70% Mobile Phase A/30% Mobile Phase B until a stable baseline is obtained. Optionally, inject reference solution for the purpose of column conditioning until a visual evaluation of the chromatograms demonstrates consistent profiles for at least two consecutive injections.

Operating Parameters
Detection wavelength: 280 nm
Injection volume: see below Injection Protocol
Flow rate: 0.3 mL/min
Column temperature: 70° C.±2° C.
Autosampler temperature: 10° C.±4° C.
Run time 30 min

Gradient

TABLE 15
BINARY GRADIENT

| Time (min) | Mobile Phase A (%) | Mobile Phase B (%) |
|---|---|---|
| 0 | 70 | 30 |
| 15 | 55 | 45 |
| 20 | 10 | 90 |
| 21 | 70 | 30 |
| 30 | 70 | 30 |

Injection Protocol

For each dosage form, separate sequences have to be performed using the corresponding reference standard. The injections of the samples are performed in the order shown in Table 16.

TABLE 16
INJECTION PROTOCOL

| Sample Type | Description | Injection Volume (μL) |
|---|---|---|
| Blank | Drug product dilution buffer | 10 |
| Standard Curve | Reference A solution | 6 |
| | Reference A solution | 8 |
| | Reference A solution | 10 |
| | Reference A solution | 12 |
| | Reference A solution | 14 |
| Assay Control | Reference B solution | 10 |
| Sample | Sample solution (1 to n) | 10 |
| Assay Control | Reference B solution | 10 |
| Blank | Drug product dilution buffer | 10 |

Note:
For more than 10 samples, bracket every 10 samples injections with control solution (Reference B).

Results

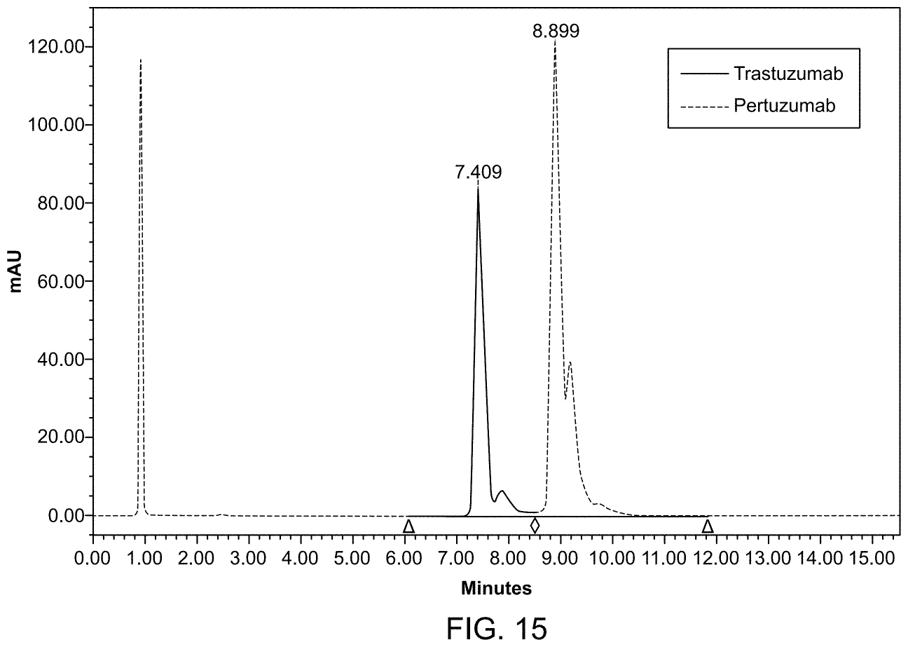

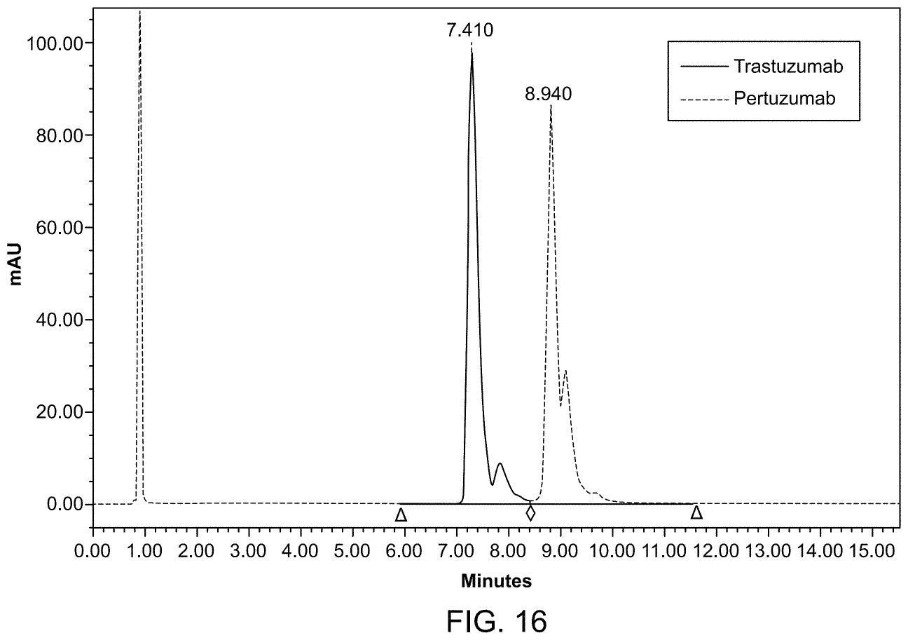

Typical chromatographic profiles are shown in FIG. 15 for FDC drug product LD and in FIG. 16 FDC drug product MD.

With the final method (example 18), substantial improvement of the initial protein content method had been obtained, including an improved overall resolution/peak separation and elimination of sample carryover, i.e. carryover does not exceed 0.2% in the subsequent analysis. Further the final method allows a quantitative protein content determination for Pertuzumab and Trastuzumab in maintenance and loading dose. A different phenyl-based RP column showed an improved specificity in regard to the two antibodies, only minor sample carryover was detected and allowed for accurate protein content determination. The final reversed-phase U-HPLC method for protein content determination in Pertuzumab/Trastuzumab FDC separates the two molecules at 70° C. on a phenyl-based reversed-phase column (Agilent Zorbax RRHD 300-Diphenyl) using a water—2-propanol/acetonitrile gradient and 0.1% TFA.

FIG. 15 depicts an example RP-UHPLC chromatogram to analyze protein content of FDC LD Reference Standard, FIG. 16 depicts example RP-UHPLC chromatogram to analyze protein content of FDC MD Reference Standard.

Data Analysis

Integrate the pertuzumab and trastuzumab peaks in the chromatograms of the Reference A and B solutions and in the sample solutions. The integration is defined with the aid of the representative chromatograms in FIG. 15 for FDC drug product LD and in FIG. 16 for FDC drug product MD. Generate a standard curve for each antibody by plotting the peak area versus the injected amount (μg) for each standard level. Fit the standard curve data using a linear regression. Do not force the curve through zero.

Using the standard curve equation, calculate the pertuzumab and trastuzumab amounts using the respective peak area for each sample solution and Reference B injection.

$$Amount ( sample ) = \frac{Peak area count - Y ‐ intercept}{Slope calibration curve}$$

Slope Calibration Curve

For calculating the pertuzumab and trastuzumab contents, the amount is divided by the respective injection volume and multiplied with the dilution factor

$$Content ( sample ) = \frac{Amount sample \times Dilution factor}{Injection volume}$$

### Example 19: HI-HPLC to Determine Content of FDC

Hydrophobic interaction chromatography (HI-HPLC) was evaluated. HI-HPLC is a common method for antibody analysis, in particular to identify their molecular variants, such as post-translational modifications or antibody-drug conjugate species. Additionally, it is possible to identify misfolded proteins or conformational changes, as HI-HPLC is a non-denaturing chromatographic method.

Compared to RP-UHPLC, the major differences to HI-HPLC are:
HI-chromatography is non-destructive and protein remains folded
Due to native protein folding, protein-column interactions arise only from amino acids located on the proteins surface.
Elution is not facilitated by increasing organic solvent concentration but decreasing the amount of e g ammonium sulfate to weaken hydrophobic-hydrophobic interactions between the protein and stationary phase. Less hydrophobic species therefore elute earlier.

Two columns for HIC-HPLC were tested:
TSKgel Ether-column, 75 mm×7.5 mm, 10 μm particle size
TSKgel Butyl-column, 35 mm×4.6 mm, 2.5 μm particle size

Mobile phases tested:
Eluent A: 50 mM sodium phosphate, pH 7.0±0.05, 5% (v/v) Ethanol
Eluent B: 50 mM sodium phosphate, 2 M ammonium sulfate, pH 7.0±0.05

Results:

HI-HPLC is able to separate the molecules of Pertuzumab/Trastuzumab FDC with either column type. The Butyl column has a far superior resolution compared to the Ether column for Coformulation samples (data not shown). In terms of RP-UHPLC and HI-HPLC comparison, in particular for protein content analysis, RPUHPLC was preferred over HI-HPLC. HI-chromatography separated the two antibodies but lacked overall resolution and showed pronounced tailing effects. Reversed-phase chromatography shows an improved resolution of Pertuzumab and Trastuzumab over HI-HPLC In particular, shoulder peaks of Pertuzumab and Trastuzumab are better resolved on RPC than HIC. Furthermore, in RPC results in a horizontal baseline which is preferred over the slanted baseline in HIC. Additionally, using a water-organic solvent gradient is less strenuous on the HPLC system than a high-low salt gradient.

TABLE 18
WORKING CONDITIONS AND HIC GRADIENT
FOR HI-HPLC TEST METHOD

| Flow rate | 0.5 mL/min | |
|---|---|---|
| Maximum pressure | 20 bar/290 psi | |
| Wavelength | 214 nm | |
| Runtime | 55 min | |
| Column temperature | 25° C. ± 3° C. | |
| Sample temperature | 5° C. ± 3° C. | |
| Sample concentration | n.a. | |
| Injection volume | 10 μL | |
| Gradient | Minutes | % Eluent B |
| | 0 | 70 |
| | 5 | 70 |
| | 35 | 0 |
| | 40 | |
| | 45 | 70 |
| | 55 | 70 |

While certain embodiments of the present invention have been shown and described herein, it will be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A binding assay for a fixed dose combination (FDC) of two anti-HER2 antibodies comprising:
a. contacting the FDC with a capture reagent comprising a modified HER2 ECD subdomain;
b. contacting the sample with a detectable antibody; and
c. quantifying the level of antibody bound to the capture reagent using a detection means for the detectable antibody.

2. The binding assay of claim 1, wherein the fixed dose combination comprises an antibody binding to HER2 extracellular subdomain II and an antibody binding to HER2 extracellular subdomain IV.

3. The binding assay of claim 1, wherein the binding of an antibody binding to HER2 extracellular subdomain II is quantified.

4. The binding assay of claim 1, wherein the capture reagent comprises a recombinant HER2 extracellular domain II.

5. The binding assay of claim 4, wherein the capture reagent comprises SEQ ID NO: 2 or SEQ ID NO: 23.

6. The binding assay of claim 1, wherein the capture reagent comprises recombinant HER2 extracellular domains I, II, III.

7. The binding assay of claim 6, wherein the capture reagent comprises SEQ ID NO: 24.

8. The binding assay of claim 3, wherein the capture reagent does not comprise a HER2 subdomain IV.

9. The binding assay of claim 1, wherein the binding of an antibody binding to HER2 subdomain IV is quantified.

10. The binding assay of claim 9, wherein the capture reagent comprises recombinant HER2 extracellular domain IV.

11. The binding assay of claim 10, wherein the capture reagent comprises SEQ ID NO: 4 or SEQ ID NO: 28.

12. The binding assay of claim 9, wherein the capture reagent does not comprise a HER2 subdomain II.

13. The binding assay of claim 9, wherein the capture reagent comprises recombinant HER2 extracellular domains I, III, IV and domain II of EGFR.

14. The binding assay of claim 9, wherein the capture reagent comprises SEQ ID NO. 29.

15. The binding assay of claim 1, for analyzing the potency of one of the anti-HER2 antibodies.

16. The binding assay of claim 15, wherein potency is quantified by correlating the level of antibody bound to the capture reagent with the biological activity of the isolated antibodies measured in a cell-based assay.

17. The binding assay of claim 1, wherein the capture reagent is coated on a microtiter plate.

18. The binding assay of claim 1, wherein the detectable antibody targets the F(ab′)2 portion of the anti-HER2 antibody

19. The binding assay of claim 1, wherein the fixed dose combination additionally comprises hyaluronidase.

20. An isolated protein comprising SEQ ID NO: 24.

21. An isolated protein comprising SEQ ID NO. 29.

22. A kit for specifically quantifying the binding of an antibody binding to HER2 extracellular subdomain II in a fixed dose combination (FDC) of a first antibody binding to HER2 extracellular subdomain II and a second anti-HER2 antibody, comprising:
a. a container containing, as a capture reagent, a protein comprising SEQ ID NO: 1, SEQ ID NO: 2 and SEQ ID NO: 34; and
b. instructions for quantifying the binding of an antibody binding to HER2 extracellular subdomain II.

23. A kit for specifically quantifying the binding of an antibody binding to HER2 extracellular subdomain IV in a fixed dose combination (FDC) of an antibody binding to HER2 extracellular subdomain IV and a second anti-HER2 antibody, the kit comprising:
a. a container containing, as a capture reagent, a protein comprising SEQ ID NO: 33, SEQ ID NO: 36, SEQ ID NO: 3 and SEQ ID NO: 4; and
b. instructions for quantifying the binding of an antibody binding to HER2 extracellular subdomain IV.

24. A method for evaluating a fixed dose composition comprising Pertuzumab and Trastuzumab, said method comprising:
a. Binding the antibodies to a ion exchange material using a loading buffer, wherein the pH of the loading buffer is between about pH 7.5 and about pH 7.65; and
b. Eluting the antibodies with an elution buffer, wherein the pH of the elution buffer is between about pH 7.5 and about pH 7.7.

25.-33. (canceled)

34. A method for making a composition is provided, comprising: (1) producing a fixed dose composition comprising pertuzumab, trastuzumab and one or more variants thereof, and (2) subjecting the composition so-produced to an analytical assay to evaluate the amount of the variant(s) therein, wherein the variant(s) comprise: (i) pertuzumab deamidated at HC-Asn-391, pertuzumab FC sialic acid variant, and pertuzumab lysine glycation variant (ii) pertuzumab native antibody, (iii) trastuzumab native antibody, or (vi) trastuzumab with single isomerization of HC-Asp-102 to iso-aspartic acid at one heavy chain.

35.-37. (canceled)

38. A composition comprising Pertuzumab and Trastuzumab, comprising less than 23% of acidic pertuzumab variants selected from deamidation of HC-Asn-391, Fc sialic acid, and lysine glycation and trastuzumab variants deamidated at LC-Asn-30 and trastuzumab variants deamidated at HC-Asn-55, at least 28% of Pertuzumab native antibody, at least 16% of Trastuzumab native antibody and less than 12% trastuzumab with single isomerization of HC-Asp-102 to iso-aspartic acid at one heavy chain.

39. The composition of claim 38, comprising less than 23% of acidic pertuzumab variants selected from deamidation of HC-Asn-391, Fc sialic acid, and lysine glycation and trastuzumab variants deamidated at LC-Asn-30 and trastuzumab variants deamidated at HC-Asn-55, at least 38% of Pertuzumab native antibody, at least 16% of Trastuzumab native antibody and less than 9% trastuzumab with single isomerization of HC-Asp-102 to iso-aspartic acid at one heavy chain.

40. The composition of claim 38, comprising less than 21% of acidic pertuzumab variants selected from deamidation of HC-Asn-391, Fc sialic acid, and lysine glycation and trastuzumab variants deamidated at LC-Asn-30 and trastuzumab variants deamidated at HC-Asn-55, at least 28% of Pertuzumab native antibody, at least 23% of Trastuzumab native antibody and less than 12% trastuzumab with single isomerization of HC-Asp-102 to iso-aspartic acid at one heavy chain.

41. A composition comprising Pertuzumab and Trastuzumab, comprising less than 23% peak area for the sum of peaks 1 to 3, at least 28% peak area for peak 4 (Pertuzumab native antibody), at least 16% peak area for peak 7 (Trastuzumab native antibody) and less than 12% peak area for peak 8.

42. The composition of claim 41, comprising less than 23% peak area for the sum of peaks 1 to 3, at least 38% peak area for peak 4 (Pertuzumab native antibody), at least 16% peak area for peak 7 (Trastuzumab native antibody) and less than 9% peak area for peak 8.

43. The composition of claim 41, comprising less than 21% peak area for the sum of peaks 1 to 3, at least 28% peak area for peak 4 (Pertuzumab native antibody), at least 23% peak area for peak 7 (Trastuzumab native antibody) and less than 12% peak area for peak 8.

44. The composition of claim 38, additionally comprising rHuPH20.

45. The composition of claim 38, comprising 40 to 60 mg/mL Trastuzumab and 60-80 mg/mL Pertuzumab.

46. The composition of claim 38, obtainable by:
a. adding a pre-defined amount of pertuzumab to a compounding vessel
b. adding trastuzumab in a 1:1 Trastuzumab to Pertuzumab ratio or in a 1:2 Trastuzumab to Pertuzumab ratio; and
c. adding rHuPH20.

47. A method for analyzing the protein content of a fixed dose combination (FDC) of two anti-HER2 antibodies comprising:
a. Providing a RP-HPLC phenyl column;
b. Loading the fixed dose combination (FDC) of two anti-HER2 antibodies on the RP-HPLC column; and
c. Separating the two anti-HER2 antibodies at a flow rate of 0.2-0.4 mL/min, wherein the column temperature is 64° C. to 76° C.

48.-57. (canceled)


SEQUENCE LISTING (36 sequences)

SEQ ID NO: 1 - protein, 195 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTR

SEQ ID NO: 2 - protein, 124 aa, Homo sapiens
TVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARV

SEQ ID NO: 3 - protein, 169 aa, Homo sapiens
CYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLA

SEQ ID NO: 4 - protein, 142 aa, Homo sapiens
CHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 5 - protein, 107 aa, Mus musculus
DTVMTQSHKIMSTSVGDRVSITCKASQDVSIGVAWYQQRPGQSPKLLIYSASYRYTGVPDRFTGSGSGTDFTFTISSVQAEDLAVYYCQQYYIYPYTFGGGTKLEIK

SEQ ID NO: 6 - protein, 119 aa, Mus musculus
EVQLQQSGPELVKPGTSVKISCKASGFTFTDYTMDWVKQSHGKSLEWIGDVNPNSGGSIYNQRFKGKASLTVDRSSRIVYMELRSLTFEDTAVYYCARNLGPSFYFDYWGQGTTLTVSS

SEQ ID NO: 7 - protein, 107 aa, synthetic construct [sequence omitted]

SEQ ID NO: 8 - protein, 119 aa, synthetic construct [sequence omitted]

SEQ ID NO: 9 - protein, 107 aa, synthetic construct
  REGION 1..107: note = source = /note="Description of Artificial Sequence: Synthetic polypeptide"
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIK

SEQ ID NO: 10 - protein, 119 aa, synthetic construct
  REGION 1..119: note = source = /note="Description of Artificial Sequence: Synthetic polypeptide"
EVQLVESGGGLVQPGGSLRLSCAASGFTFSSYAMSWVRQAPGKGLEWVAVISGDGGSTYYADSVKGRFTISRDNSKNTLYLQMNSLRAEDTAVYYCARGRVGYSLYDYWGQGTLVTVSS

SEQ ID NO: 11 - protein, 214 aa, synthetic construct
  REGION 1..214: note = source = /note="Description of Artificial Sequence: Synthetic polypeptide"
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 448 aa, synthetic construct
  REGION 1..448: note = source = /note="Description of Artificial Sequence: Synthetic polypeptide"
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 214 aa, synthetic construct
  REGION 1..214: note = source = /note="Description of Artificial Sequence: Synthetic polypeptide"
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 449 aa, synthetic construct
  REGION 1..449: note = source = /note="Description of Artificial Sequence: Synthetic polypeptide"
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 217 aa, synthetic construct
  REGION 1..217: note = source = /note="Description of Artificial Sequence: Synthetic polypeptide"
VHSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 449 aa, synthetic construct
  REGION 1..449: note = source = /note="Description of Artificial Sequence: Synthetic polypeptide"
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 10 aa, synthetic construct
  VARIANT 10: note = /replace="Ser"
  SITE 10: note = MISC_FEATURE - /note=" Residue given in the sequence has no preference with respect to that in the annotations for said position "
  REGION 1..10: note = source = /note="Description of Artificial Sequence: Synthetic peptide"
GFTFTDYTMD

SEQ ID NO: 18 - protein, 17 aa, synthetic construct
  REGION 1..17: note = source = /note="Description of Artificial Sequence: Synthetic peptide"
DVNPNSGGSIYNQRFKG

SEQ ID NO: 19 - protein, 10 aa, synthetic construct
  REGION 1..10: note = source = /note="Description of Artificial Sequence: Synthetic peptide"
NLGPSFYFDY

SEQ ID NO: 20 - protein, 11 aa, synthetic construct
  REGION 1..11: note = source = /note="Description of Artificial Sequence: Synthetic peptide"
KASQDVSIGVA

SEQ ID NO: 21 - protein, 7 aa, synthetic construct
  VARIANT 5: note = /replace="Leu"
  VARIANT 6: note = /replace="Glu"
  VARIANT 7: note = /replace="Ser"
  REGION 5..7: note = MISC_FEATURE - /note="Residue given in the sequence has no preference with respect to that in the annotations for said position "
  REGION 1..7: note = source = /note="Description of Artificial Sequence: Synthetic peptide"
SASYRYT

SEQ ID NO: 22 - protein, 9 aa, synthetic construct
  REGION 1..9: note = source = /note="Description of Artificial Sequence: Synthetic peptide"
QQYYIYPYT

SEQ ID NO: 23 - protein, 119 aa, Homo sapiens
TVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSK

SEQ ID NO: 24 - protein, 483 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECV

SEQ ID NO: 25 - protein, 725 aa, synthetic construct
  REGION 1..725: note = Capture reagent for anti-HER2 antibody
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVRRAQVTDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 26 - protein, 756 aa, synthetic construct
  REGION 1..756: note = capturing reagent for anti-HER2 antibody
KYALADASLKMADPNRFRGKDLPVLDQLLESTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVRRAQVTDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 27 - protein, 757 aa, synthetic construct
  REGION 1..757: note = Capturing agent for anti-HER2 antibody
KYALADASLKMADPNRFRGKDLPVLDQLLESTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVRRAQVTDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 28 - protein, 136 aa, Homo sapiens
CHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQ

SEQ ID NO: 29 - protein, 624 aa, synthetic construct
  REGION 1..624: note = Capturing reagent for anti-HER2 antibody
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQ

SEQ ID NO: 30 - protein, 866 aa, synthetic construct
  REGION 1..866: note = Capturing reagent for anti-HER2 antibody
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRRAQVTDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 31 - protein, 899 aa, synthetic construct
  REGION 1..899: note = Capturing reagent for anti-HER2 antibody
KYALADASLKMADPNRFRGKDLPVLDQLLEAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRRAQVTDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 32 - protein, 900 aa, synthetic construct
  REGION 1..900: note = Capturing reagent for anti-HER2 antibody
KYALADASLKMADPNRFRGKDLPVLDQLLEAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRRAQVTDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 33 - protein, 191 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQ

SEQ ID NO: 34 - protein, 164 aa, Homo sapiens
CYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECV

SEQ ID NO: 35 - protein, 242 aa, synthetic construct
  REGION 1..242: note = Fc domain
RRAQVTDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 36 - protein, 128 aa, Homo sapiens
KLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKV